(19) United States
(12) Patent Application Publication
(10) Pub. No.: US 2003/0148382 A1
(43) Pub. Date: Aug. 7, 2003

(54) PanCAM nucleic acids and polypeptides

(75) Inventors: Chao Sun, Needham, MA (US); John P. Carulli, Southborough, MA (US); Alexander V. Lukashin, Charlestown, MA (US); Daniel R. Kilburn, Lexington, MA (US)

Correspondence Address: FISH & NEAVE, 1251 AVENUE OF THE AMERICAS, 50TH FLOOR, NEW YORK, NY, 10020-1105, US

(73) Assignee: Biogen, Inc.

(21) Appl. No.: 10/180,410

(22) Filed: Jun. 24, 2002

Related U.S. Application Data
(63) Continuation-in-part of application No. PCT/US01/19904, filed on Jun. 22, 2001.
(60) Provisional application No. 60/213,611, filed on Jun. 22, 2000.

(51) Int. Cl. G01N 33/53; C07H 21/04; C12P 21/02; C12N 5/06; C07K 16/40

(52) U.S. Cl. 435/7.1; 435/69.1; 435/326; 435/320.1; 530/388.1; 536/23.53

(57) ABSTRACT

An isolated polynucleotide encoding a novel immunoglobulin superfamily member named PanCAM (also named GP354) is provided. PanCAM has a predicted single membrane spanning domain and five immunoglobulin (Ig) domains in the extracellular portion of the protein. The protein structure and tissue distribution of PanCAM indicate that it plays a role in cell-cell recognition, binding, signaling and adhesion events in the pancreas (especially the beta islets), the pituitary gland, and the central nervous system (CNS). Provided by the invention are isolated PanCAM related polynucleotides and polypeptides, vectors, and host cells comprising any of the above, antibodies directed to PanCAM, cells which produce such antibodies, and related diagnostic and therapeutic methods.

### RELATED APPLICATIONS

This application claims priority from PCT/JUS01/19904, filed Jun. 22, 2001, which claims priority from U.S. Provisional Application No. 60/213,611, filed Jun. 22, 2000. The disclosures of both the PCT and provisional applications are incorporated herein by reference in their entirety.

### FIELD OF THE INVENTION

The present invention relates generally to the field of molecular biology. More particularly, this invention relates to members of the immunoglobulin superfamily.

### BACKGROUND OF THE INVENTION

Many proteins have been classified into superfamilies based on conserved structural motifs and biological functions. A superfamily is broadly defined as a group of proteins that share a certain degree of sequence homology, usually at least 15%. The conserved sequences shared by superfamily members often contribute to the formation of compact tertiary structures referred to as domains, and often the entire sequence of a domain characteristic of a particular superfamily is encoded by a single exon (see, e.g., Abbas et al., CELLULAR AND MOLECULAR IMMUNOLOGY, W.B. Saunders Co., Philadelphia, Pa. 1997). Members of a superfamily are likely derived from a common precursor gene by divergent evolution, and multidomain proteins may belong to more than one superfamily. Examples of protein superfamilies include the ligand-gated ion channel receptor superfamily, the voltage-dependent ion channel receptor superfamily, the receptor tyrosine kinase superfamily, the receptor protein tyrosine phosphatase superfamily, the G protein-coupled receptor superfamily, and the immunoglobulin (Ig) superfamily.

The Ig superfamily encompasses proteins that share partial amino acid sequence homology and tertiary structural features that were originally identified in Ig heavy and light chains. The common structural motif of the Ig superfamily is the so-called “Ig domain.” Ig domains are three-dimensional globular structures having about 70 to 110 amino acid residues and an internal Cys-Cys disulfide bond. These domains contain two layers of β-pleated sheet, each layer composed of three to five antiparallel strands of five to ten amino acid residues. Ig domains are classified as V-like or C-like on the basis of closest homology to either the Ig V or C domains. For a general review, see, e.g., Abbas et al., supra.

Most identified members of the Ig superfamily are integral plasma membrane proteins with Ig domains in the extracellular portions and widely divergent cytoplasmic tails, usually with no intrinsic enzymatic activity. One recurrent characteristic of the Ig superfamily members is that interactions between Ig domains on different polypeptide chains (of the same or different amino acid sequences) are essential for the biological activities of the molecules. Heterophilic interactions can also occur between Ig domains on entirely distinct molecules expressed on the surfaces of different cells. Such interactions provide adhesive forces that stabilize cell-cell binding.

Many members of the Ig superfamily are cell surface or soluble molecules that mediate cell recognition, adhesion and binding functions in the vertebrate immune system. Two prominent cell types that produce Ig superfamily molecules are B and T lymphocytes. Exemplary Ig superfamily member proteins of importance in the immune system include antibodies, T cell receptors, Class I and II major histo-compatibility complex (MHC) molecules, CD2, CD3, CD4, CD5, CD8, CD28, CD20 (B1), CD32 (FcgRII), CD44, CD54 (ICAM-1), CD80 (B7-1), CD86 (B7-2), CD90 (Thy-1), CD102 (ICAM-2), CD106 (VCAM-1), CD121 (IL-IR), CD152 (CTLA-4), p-IgR, NCAM, and CD140 (PDGFR) (Abbas et al., supra).

Several Ig superfamily members have been identified outside the immune system, for instance, in the nervous system. Based on their conserved structural motifs and the well known functions of such motifs in the immune system, these Ig superfamily members likely perform cell recognition, binding and adhesion functions in non-immune tissues as well. Novel Ig superfamily members localized to particular cell types will be useful cell and tissue markers for diagnostic purposes. Tissue specific Ig superfamily members will also be suitable therapeutic targets for treating abnormal conditions, disorders and/or diseases related to improper cell-cell adhesion and signaling in the tissue, particularly during tissue development or during tissue regeneration, e.g., after tissue damage or trauma.

### SUMMARY OF THE INVENTION

The present invention is based, at least in part, on the discovery of a gene encoding a heretofore unknown Ig superfamily member, termed GP354. Unless indicated otherwise, the name in lower case, gp354, refers to the new nucleic acids of the invention, whereas the name in uppercase, GP354, refers to the new polypeptides of the present invention.

GP354 is an integral membrane protein predominantly expressed in the pancreas. This protein is also named PanCAM, for pancreas cell adhesion molecule. In the pancreas, GP354 (i.e., PanCAM) expression is localized to β cells. GP354 is also detected in low levels in the central nervous system (CNS), such as the brain. GP354 has a predicted single membrane spanning domain and five extracellular Ig domains. Human GP354 shares about 44% identity to a 494 amino acid fragment of human NEPH1 and 47% amino acid identity with a 368 amino acid fragment of mouse NEPH1. Both fragments are in the extracellular portion of NEPH1. GP354 is likely to play a role in cell-cell interactions (e.g., adhesion) in the pancreas and the CNS, affecting these organs' structures and functions.

The invention provides isolated polynucleotides encoding a vertebrate GP354 (e.g., mammalian such as human or mouse) or a biologically active portion thereof. This invention also provides polynucleotide fragments suitable for use as primers or hybridization probes for the detection of GP354-encoding polynucleotides. Even though this disclosure exemplifies particular GP354 or gp354 sequences, all allelic variations of these sequences (e.g., polymorphism) are encompasses by this invention.

In some embodiments, the invention provides an isolated human GP354 polypeptide comprising amino acid residues 68-594 of SEQ ID NO:12. Such an isolated polypeptide may comprise SEQ ID NO:12, 26, 28, or 8. The polypeptide may further comprises a heterologous sequence. The invention also provides an isolated mouse PanCAM polypeptide comprising SEQ ID NO:31.

The invention also provides an isolated polypeptide comprising an extracellular domain of a PanCAM protein, for instance, comprising amino acid residues 4-513 of SEQ ID NO:8, or a portion thereof having at least 10 amino acid (e.g., 25, or 50) residues. This polypeptide can be used for raising PanCAM-specific antibodies or used as PanCAM modulators.

The invention also provides an isolated polypeptide comprising an intracellular domain of a PanCAM protein, for instance, comprising amino acid residues 535-708 of SEQ ID NO:8, or amino acid residues 535-633 of SEQ ID NO:26, or a portion thereof having at least 10 amino acid (e.g., 25, or 50) residues. This polypeptide can be conjugated to a heterologous extracellular domain to form a hybrid molecule. The hybrid molecule can transmit a PanCAM-like intracellular signal via stimulation of the nonPanCAM extracellular domain.

The invention provides isolated polynucleotides comprising nucleotide sequences encoding the above-described isolated polypeptides. Exemplary isolated polynucleotides are those comprising part (e.g., nucleotides 202-1782) or all of SEQ ID NO:11; part (e.g., nucleotides 196-2319) or all of SEQ ID NO:7; and part or all of SEQ ID NOs:1, 3, 5, 6, 9, 25, 27, and 32. In some embodiments, the polynucleotides further comprise a transcription regulatory sequence operatively linked to said nucleotide sequence.

Also included in this invention are vectors comprising these isolated polynucleotides. The vectors may be plasmid vectors, or viral vectors derived from baculoviruses, adenoviruses, parvoviruses, herpesviruses, poxviruses, adeno-associated viruses, Semliki Forest viruses, vaccinia viruses, lentiviruses and retroviruses.

The invention also includes host cells harboring the isolated polynucleotides of this invention. The host cells can be, e.g., bacterial cells, insect cells, yeast cells, plant cells or mammalian cells (e.g., mouse cells, or human cells). In some embodiments, these host cells can be used to produce a PanCAM polypeptide. To do this, the host cells are cultured under conditions in which the PanCAM-encoding sequence is expressed, and recovering the polypeptide from the cells or from the culture medium.

This invention also features antibodies that binds specifically to a PanCAM protein. The antibodies can be whole antibodies or fragments thereof (e.g., Fab fragments, F(ab′)2 fragments). They can also be singled-chained antibodies, chimeric antibodies, humanized and fully human antibodies.

The invention also features a method of isolating beta cells from a dispersed pancreatic cell population (e.g., a bovine or swine pancreatic cell population). This method comprises the steps of providing an antibody specific for a PanCAM protein; contacting the dispersed pancreatic cell population with the antibody under conditions that allow beta cells in the population to bind to the antibody; and separating cells unbound by the antibody from beta cells bound to the antibody, thereby isolating beta cells.

The beta cells so isolated can be used to treat a human displaying a sign or symptom of diabetes or a pre-diabetic condition. This treatment method comprise the steps of introducing the isolated beta cells into the human, e.g., subcutaneously. The beta cells can be encapsulated in a biological acceptable carrier such as alginate spheres (see, e.g., Ryu et al., J. Clin. Invest. 2001, 108(1): 31-3).

This invention further features a polynucleotide that includes a nucleotide sequence which encodes a protein that comprises an amino acid sequence that is at least 80% (85%, 95% or 98%) identical to the amino acid sequence of SEQ ID NO:2 (encoded by a predicted gp354 cDNA); SEQ ID NO:4 (encoded by a partial gp354 pancreatic cDNA); SEQ ID NO:8 (encoded by a derived gp354 cDNA); SEQ ID NO:10 (encoded by a partial derived gp354 cDNA); or SEQ ID NO:12 (encoded by a gp354 pancreatic cDNA, an insert of plasmid FL86-3, which was deposited at American Type Culture Collection (ATCC) on Jun. 11, 2002); or to at least one Ig domain of any one of SEQ ID NOs:2, 4, 8, 10 and 12.

In some embodiments, the polynucleotide comprises the sequence of SEQ ID NO:1 (a gp354 cDNA), or a fragment thereof having at least 17 nucleic acid units (e.g., nucleotides). An example of such a fragment is SEQ ID NO:3. In another embodiment, a polynucleotide comprises the sequence of SEQ ID NO:5 (genomic DNA comprising gp354), or a fragment thereof having at least 17 nucleic acid units. An exemplary fragment is that of SEQ ID NO:6 (gp354 upstream genomic DNA). In other embodiments, a polynucleotide comprises the sequence of SEQ ID NO:7 (a derived gp354 cDNA), or a fragment thereof having at least 17 nucleic acid units. An exemplary fragment is that of SEQ ID NO:9 (C-terminal fragment of a derived gp354 cDNA). In other embodiments, a polynucleotide comprises the sequence of SEQ ID NO:11 (pancreatic gp354 cDNA), or a fragment thereof having at least 17 nucleic acid units. Preferred fragments encode part or all of at least one extracellular Ig domain and/or an intracellular domain of GP354.

The invention also provides a polynucleotide which encodes a naturally occurring, allelic variant of a polypeptide comprising the amino acid sequence of SEQ ID NO:2, wherein the nucleic acid hybridizes to SEQ ID NO:1 or SEQ ID NO:11 under stringent conditions. The invention also provides a polynucleotide which encodes a naturally occurring, allelic variant of a polypeptide comprising the amino acid sequence of SEQ ID NO:4, 8, 10 or 12, wherein the nucleic acid hybridizes to SEQ ID NO:1 or 11 under stringent conditions.

Also provided by the invention is an isolated GP354 protein comprising an amino acid sequence that is at least 80% (85%, 95% or 98%) identical to the amino acid sequence of SEQ ID NO:2, 4, 8, 10 or 12; or to an Ig domain encoded by any one of those sequences.

The invention also provides an isolated GP354 protein encoded by a polynucleotide comprising a sequence which is at least about 65%, preferably 75%, 85%, or 95% identical to SEQ ID NO:1, 3, 5, 7, 9 or 11; or to a portion of any one of those sequences that encodes at least one Ig domain. Also provided is an isolated GP354 protein encoded by a polynucleotide having a sequence which hybridizes under stringent conditions to a nucleic acid having the sequence of SEQ ID NO:1 or 11.

The invention provides gp354 polynucleotides that specifically detect gp354 nucleic acids relative to nucleic acids encoding other members of the Ig superfamily. The invention also provides a nucleic acid construct, e.g., a recombinant vector (e.g., a cloning, targeting or expression vector), comprising a gp354 polynucleotide of the invention.

Isolated or recombinant GP354 proteins and polypeptides are provided by the invention. In some embodiments, GP354 proteins and polypeptides display at least one of the following (overlapping) biological activities: (1) interaction with (e.g., binding to) a ligand (e.g., a protein receptor, a polysaccharide, etc.) that naturally binds to GP354 protein; (2) binding to an auto-antibody to naturally occurring human GP354 or an antibody raised against naturally occurring human GP354; (3) participation in a pancreatic function (e.g., a signal transduction function in the pancreas or a step in the organ development of the pancreas); (4) participation in a neural function (e.g., a signal transduction function in the nervous system or step in the development of the nervous system); and (5) mediation of cell-cell interactions such as recognition, binding and/or adhesion.

The GP354 proteins or biologically active portions thereof can be operably linked to a non-GP354 polypeptide (e.g., heterologous amino acid sequences, such as sequences that facilitate protein stability, detection, purification, or in vivo delivery to target cells) to form GP354 fusion proteins.

The invention provides pharmaceutical compositions comprising at least one of the above-described gp354-related isolated polynucleotides, GP354 proteins or biologically active portions thereof, antibodies or fusion proteins; which optionally include pharmaceutically acceptable carriers. Such compositions are useful in therapeutic methods for ameliorating conditions in a subject associated with abnormal GP354 cellular localization, expression and/or activity.

As such, the present invention also provides methods of treatment comprising the step of administering a gp354-related compound or composition of the invention. Such methods will be useful, for example, for treating abnormal conditions, disorders or diseases which correlate with cell recognition, binding, signaling and adhesion functions in the developing or adult pancreas and central nervous system.

As a pancreatic enriched protein, GP354 will be a suitable therapeutic target for treating abnormal conditions, disorders and/or diseases related to improper cell-cell binding, adhesion and signaling in the developing and adult pancreas, particularly during tissue development and during tissue regeneration and/or healing, e.g., after pancreatic damage, trauma or degenerative conditions. It is also envisioned that GP354 will be a suitable therapeutic target for inhibiting pancreatic cell death associated with immune, auto-immune, and degenerative conditions (including type I and type II diabetes). The neural form of GP354 will be a similarly suitable therapeutic target for treating tissue abnormalities, for tissue regeneration and repair, and for inhibiting tissue degeneration and cell death in the central nervous system. The GP354-related compositions can also be used to treat diseases in the pituitary gland such as pituitary hormone deficiency, growth hormone deficiency, and pituitary tumors.

The invention provides a method for modulating GP354 activity. In this method, a target cell is contacted with an agent that modulates (e.g., inhibits or stimulates) GP354 activity or expression such that the GP354 activity or expression is altered. In some embodiments, the agent is an antibody that specifically binds to GP354. In other embodiments, the agent modulates the GP354 activity or expression by modulating transcription of a gp354 gene, splicing of gp354 RNA, or translation of a gp354 mRNA. In yet other embodiments, the agent is a nucleic acid having a sequence that is antisense to the coding strand of the gp354 mRNA or the gp354 gene. In other embodiments, the agent can be a GP354 protein, a nucleic acid encoding a GP354 protein, or an antagonist or agonist of the GP354 protein such as a peptide, a peptidomimetic, or other small molecules.

The invention also provides a method for identifying a compound that binds to a GP354 protein. In another aspect, the invention provides a method for identifying a compound that modulates the biological activity of a GP354 protein, comprising measuring a biological activity or expression of the protein in the presence and absence of a test compound and identifying those compounds which alter the activity of the protein. Combinatorial libraries can be used as sources of candidate compounds in these methods.

The invention provides a method for detecting the presence of a gp354 polynucleotide, a GP354 protein or its activity in a biological sample (e.g., a fluid or tissue sample derived from a patient) by contacting the sample with an agent capable of detecting an indicator of the presence of gp354 polynucleotide sequences, GP354 protein or its activity.

A diagnostic assay is provided for identifying the presence or absence of a gp354-related genetic lesion or mutation, characterized by at least one of the following: (i) aberrant modification or mutation of a gene encoding a GP354 protein; (ii) mis-regulation (e.g., transcription, splicing or translation) of a gene encoding a GP354 protein; and (iii) aberrant post-translational modification or localization of a GP354 protein; wherein the wild-type form of the gene encodes a protein with a GP354 biological activity.

The invention provides a non-human animal (e.g., a mammal such as a mouse, rat, guinea pig, sheep, goat, horse or cow) at least some cells of which comprise an isolated polynucleotide of this invention. Such an animal can be chimeric where only some of its somatic and/or germ cells carry the polynucleotide. Such an animal can alternatively be transgenic where all of its somatic and germ cells carry the polynucleotide.

The invention also provides a non-human animal whose endogenous ortholog of the gp354 gene is disrupted by gene targeting (i.e., “knocked out”). Cells containing a gp354 polynucleotide, biological samples such as tissues and fluids and GP354-related products derived from these and the above-mentioned animals are also within the scope of this invention.

The invention provides a computer readable means of storing the nucleic acid and amino acid sequences of the instant invention. The records of the computer readable means can be accessed for reading and display of sequences and for comparison, alignment and ordering of the sequences of the invention to other sequences.

Other features and advantages of the invention will be apparent from the following detailed description, drawings, and from the claims.

### BRIEF DESCRIPTION OF THE DRAWINGS

FIG. 1 The coding (SEQ ID NO:1) and deduced amino acid sequences (SEQ ID NO:2) of a human GP354 (PanCAM) polypeptide. The immunoglobulin (Ig) domains in the extracellular portion and the transmembrane domain are underlined.

FIG. 2 The alignment of the human GP354 amino acid sequence shown in FIG. 1 (SEQ ID NO:2) with sequences of Drosophila irregular chiasm (ICCR) (SEQ ID NO:13) and human nephrin (SEQ ID NO:14) proteins. Dashes indicate gaps in any of the sequences. Asterisks denote amino acids that are identical in the three sequences.

FIG. 3 Sequence of the RT-PCR fragment obtained using primers GX1-218 (SEQ ID NO:16) and GX1-219 (SEQ ID NO:17). This fragment (SEQ ID NO:3) is an insert of plasmid CS0026, which was deposited at ATCC on Jun. 11, 2002.

FIG. 4 A human genomic gp354 sequence (SEQ ID NO:5). Exons are underlined.

FIG. 5 The cDNA (SEQ ID NO:7) and derived amino acid sequences (SEQ ID NO:8) of a human PanCAM. SEQ ID NO:7 is generated based on SEQ ID NO:12 and the coding sequences for PanCAM-1 (an insert of plasmid CS0067-1, which was deposited at ATCC on Jun. 11, 2002 (SEQ ID NO:25)) and for PanCAM-2 (an insert of plasmid CS0067-3, which was deposited at ATCC on Jun. 11, 2002 (SEQ ID NO:27)). SEQ ID NO:7 includes 5′ and 3′ untranslated regions.

FIG. 6 The nucleotide (SEQ ID NO:11) and deduced amino acid sequences (SEQ ID NO:12) of a pancreatic gp354 cDNA.

FIG. 7 The nucleotide sequence (SEQ ID NO:25; an insert of plasmid CS0067-1) of a representative GP354 cDNA clone. The deduced amino acid sequence (SEQ ID NO:26; termed PanCAM-1) is shown below the corresponding nucleotide sequence. The signal peptide is double-underlined, the transmembrane domain is boxed, and the five Ig domains are underlined. The sequences corresponding to oligonucleotides used for amplification of the original ab initio gene prediction are underscored with thick lines, and they bracket SEQ ID NO:11.

FIG. 8 Nucleotide (SEQ ID NO:27) and deduced amino acid (SEQ ID NO:28) sequences of PanCAM-2.

FIG. 9A Alignment of proteins encoded by alternatively spliced transcripts of PanCAM: PanCAM-1 (SEQ ID NO:26), PanCAM-2 (SEQ ID NO:28), PanCAM (SEQ ID NO:8), and bc007312 (SEQ ID NO:29). The conserved Ig domains are underlined.

FIG. 9B A diagram of the PanCAM locus and alternatively spliced mRNAs. Exons are numbered 1-15 and shown as filled boxes, and exons with cryptic splicing sites are marked with asterisks. The shaded area in the PanCAM locus represents a cryptic intron, which is a portion of an exon spliced out in PanCAM-1 and PanCAM-2 transcripts.

FIG. 10A Nucleotide (SEQ ID NO:32) and deduced amino acid (SEQ ID NO:31) sequences of a partial mouse PanCAM cDNA clone.

FIG. 10B Alignment of a partial protein sequence of human PanCAM (SEQ ID NO:30) with a partial protein sequence of murine PanCAM (SEQ ID NO:31). Immunoglobulin domains are underlined. The mouse sequence is at the bottom.

### DETAILED DESCRIPTION OF THE INVENTION

The present invention is based, at least in part, on the discovery of a novel human gene encoding a heretofore unknown protein, GP354 (i.e., PanCAM). This gene, gp354, was identified by computational analysis of (“mining”) the published nucleic acid sequences of the human genome. The gp354 gene contains at least 14 exons and normally resides on human chromosome 19. An mRNA transcribed from this gene has an open reading frame of 1779 base pairs (including the stop codon) and encodes a protein predicted to be 592 amino acid residues. The novel GP354 protein is specifically expressed in the pancreas and the brain.

### Definitions

As used herein, “nucleic acid” (also “polynucleotide”) includes DNA molecules (e.g., cDNA or genomic or synthetic DNA) and RNA molecules (e.g., mRNA). The term also is intended to include analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation. See, e.g., Baner et al., Curr. Opin. Biotechnol. 12:11-15 (2001); Escude et al., Proc. Natl. Acad. Sci. USA 14;96(19):10603-7 (1999); Nilsson et al., Science 265(5181):2085-8 (1994); Praseuth et al., Biochim. Biophys. Acta. 1489(1):181-206 (1999); Fox, Curr. Med. Chem. 7(1):17-37 (2000); Kochetkova et al., Methods Mol. Biol. 130:189-201 (2000); Chan et al., J. Mol. Med. 75(4):267-82 (1997).

As used herein, an “isolated nucleic acid” (also “isolated polynucleotide”) is one which is separated from other nucleic acid molecules that are present in the natural source of the nucleic acid. Specifically excluded are isolated, non-recombinant native chromosomes and fragments thereof that are larger than 500 kilobases. Preferably, an “isolated” nucleic acid is substantially free of sequences that naturally flank that nucleic acid in the genome of the organism from which the nucleic acid is derived. For example, a preferred isolated gp354 nucleic acid is flanked by less than about 10 kb, 5 kb, 4 kb, 3 kb, 2 kb, 1 kb, 0.5 kb or 0.1 kb of nucleotide sequences that naturally flank the nucleic acid in the genomic DNA of the cell from which the isolated nucleic acid is derived. Even more preferably, the isolated polynucleotides are no more than 5000 base pairs, often no more than 1000 base pairs, 500 base pairs, 100 base pairs or 50 base pairs.

However, “isolated” does not necessarily require that the nucleic acid or polynucleotide so described has itself been physically removed from its native environment. For instance, an endogenous nucleic acid sequence in the genome of an organism is deemed “isolated” herein if a heterologous sequence (i.e., a sequence that is not naturally adjacent to this endogenous nucleic acid sequence) is placed adjacent to the endogenous nucleic acid sequence, such that the expression of this endogenous nucleic acid sequence is altered. By way of example, a non-native promoter sequence can be substituted (e.g., by homologous recombination) for the native promoter of a gp354 gene in the genome of a human cell, such that this gene has an altered expression pattern. This gene would now become “isolated” because it is separated from at least some of the sequences that naturally flank it.

A nucleic acid is also considered “isolated” if it contains any modifications that do not naturally occur to the corresponding nucleic acid in a genome. For instance, an endogenous gp354-coding sequence is considered “isolated” if it contains an insertion, deletion or a point mutation introduced artificially, e.g., by human intervention. An “isolated nucleic acid” also includes a nucleic acid integrated into a host cell chromosome at a heterologous site, a nucleic acid construct present as an episome and a nucleic acid construct integrated into a host cell chromosome. Moreover, an “isolated nucleic acid” can be substantially free of other cellular material, or substantially free of culture medium when produced by recombinant techniques, or substantially free of chemical precursors or other chemicals when chemically synthesized.

A polynucleotide of the invention is considered “full-length” if it is able to encode a full-length GP354 protein.

As used herein, the phrase “degenerate variant” of a reference nucleic acid sequence encompasses nucleic acid sequences that can be translated, according to the standard genetic code, to provide an amino acid sequence identical to that translated from the reference nucleic acid sequence.

As used herein, the term “microarray” (also “nucleic acid microarray”) refers to a substrate-bound plurality of nucleic acids, hybridization to each of the bound nucleic acids being separately detectable. The substrate can be solid or porous, planar or non-planar, unitary or distributed, or in any other configuration.

As so defined, the term “microarray” includes all the devices so called or similarly called in Schena (ed.), DNA Microarrays: A Practical Approach (Practical Approach Series), Oxford University Press (1999) (ISBN: 0199637768); Nature Genet. 21(1)(suppl):1-60 (1999); and Schena (ed.), Microarray Biochip: Tools and Technology, Eaton Publishing Company/BioTechniques Books Division (2000) (ISBN: 1881299376); Brenner et al., Proc. Natl. Acad. Sci. USA 97(4): 1665-1670 (2000). The disclosures of all of these references are incorporated herein by reference in their entireties.

As used herein with respect to nucleic acid hybridization, the term “probe” (also “nucleic acid probe” or “hybridization probe”) refers to an isolated nucleic acid of known sequence that is, or is intended to be, detectably labeled. As used herein with respect to a nucleic acid microarray, the term “probe” (or equivalently “nucleic acid probe” or “hybridization probe”) refers to the isolated nucleic acid that is, or is intended to be, bound to the substrate. In either such context, the term “target” refers to a nucleic acid intended to be bound to a probe by sequence complementarity.

Unless otherwise indicated, a “nucleic acid comprising SEQ ID NO:X” refers to a nucleic acid, at least a portion of which has either (i) the sequence of SEQ ID NO:X, or (ii) a sequence complementary to SEQ ID NO:X. The choice between the two is dictated by the context. For instance, if the nucleic acid is used as a probe, the choice between the two is dictated by the requirement that the probe be complementary to the desired target.

For purposes herein, “high stringency conditions” are defined for solution phase hybridization as aqueous hybridization (i.e., free of formamide) in 6×SSC (where 20×SSC contains 3.0 M NaCl and 0.3 M sodium citrate), 1% SDS at 65° C. for 8-12 hours, followed by two washes in 0.2×SSC, 0.1% SDS at 65° C. for 20 minutes. It will be appreciated by the skilled worker that hybridization at 65° C. will occur at different rates depending on a number of factors including the length and percent identity of the sequences which are hybridizing.

For microarray-based hybridization, standard “high stringency conditions” are defined as hybridization in 50% formamide, 5×SSC, 0.2 μg/μl poly(dA), 0.2 μg/μl human cotl DNA, and 0.5% SDS, in a humid oven at 42° C. overnight, followed by successive washes of the microarray in 1×SSC, 0.2% SDS at 55° C. for 5 minutes, and then 0.1×SSC, 0.2% SDS, at 55° C. for 20 minutes. For microarray-based hybridization, “moderate stringency conditions”, suitable for cross-hybridization to mRNA encoding structurally- and functionally-related proteins, are defined to be the same as those for high stringency conditions but with reduction in temperature for hybridization and washing to room temperature (approximately 25° C.).

As used herein, the terms “protein,” “polypeptide,” and “peptide” are used interchangeably to refer to a naturally-occurring or synthetic polymer of amino acids, irrespective of length, where amino acids here include naturally-occurring amino acids, naturally-occurring amino acid structural variants, and synthetic non-naturally occurring analogs that are capable of participating in peptide bonds. The terms “protein”, “polypeptide”, and “peptide” explicitly permit post-translational and post-synthetic modifications, such as N- or C-terminal amino acid cleavage reactions and glycosylation. The term “oligopeptide” herein denotes a protein, polypeptide, or peptide having 25 or fewer amino acid residues.

A protein, polypeptide, peptide or oligopeptide is considered “isolated” when it is encoded by an isolated polynucleotide; when it exists in a purity not found in nature, where purity can be adjudged with respect to the presence of other cellular material; and/or when it includes amino acid analogs or derivatives not found in nature or linkages other than standard peptide bonds. As thus defined, “isolated” does not necessarily require that the protein, polypeptide, peptide or oligopeptide so described has been physically removed from its native environment.

A protein, polypeptide, peptide or oligopeptide is considered “purified” herein when it is present at a concentration of at least 65% (e.g., at least 75%, 85% or 95%), as measured on a mass basis with respect to total protein in a composition. It is considered “substantially purified” when the concentration is at least 85%.

As used herein, the term “homologs” (also “homologues”) encompasses “orthologs” and “paralogs.” “Orthologs” are separate occurrences of the same gene in different species of organisms. The separate occurrences have similar or identical amino acid sequences, where the degree of sequence similarity depends in part on the evolutionary distance of the species from a common ancestor having the same gene. “Paralogs” indicates separate occurrences of a gene in one species of organism. The separate occurrences have similar or identical amino acid sequences, where the degree of sequence similarity depends in part on the evolutionary distance of these separate occurrences from the gene duplication event giving rise to the occurrences.

“Homologous” amino acid sequences include those amino acid sequences which contain conservative amino acid substitutions and which polypeptides have substantially the same binding and/or activity. A homologous amino acid sequence does not, however, include the amino acid sequence encoding other known Ig superfamily members. Homology (percent identity) can be determined by, for example, the GAP program (Wisconsin Sequence Analysis Package, Version 8 for Unix, Genetics Computer Group, University Research Park, Madison Wis.), using the default settings, which uses the algorithm of Smith and Waterman (Adv. Appl. Math., 2:482-489 (1981), which is incorporated herein by reference in its entirety).

As used herein, the term “antibody” refers to a full antibody (consisting of two heavy chains and two light chains) or a fragment thereof. Such fragments include, but are not limited to, those produced by digestion with various proteases, those produced by chemical cleavage and/or chemical dissociation, and those produced recombinantly, so long as the fragment remains capable of specific binding to an antigen. Among these fragments are Fab, Fab′, F(ab′)₂, and single chain Fv (scFv) fragments.

Within the scope of the term “antibody” are also antibodies that have been modified in sequence, but remain capable of specific binding to an antigen. Example of modified antibodies are interspecies chimeric and humanized antibodies; antibody fusions; and heteromeric antibody complexes, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513), the disclosure of which is incorporated herein by reference in its entirety).

“Specific binding” refers to the ability of two molecules to bind to each other in preference to binding to other molecules in the environment. Typically, “specific binding” discriminates over adventitious binding in a reaction by at least two-fold, more typically by at least 10-fold, often at least 100-fold. Typically, the affinity or avidity of a specific binding reaction is at least about 10⁻⁷ M (e.g., at least about 10⁻⁸ M or 10⁻⁹ M).

By the term “region” is meant a physically contiguous portion of the primary structure of a biomolecule. In the case of proteins, a region is defined by a contiguous portion of the amino acid sequence of that protein.

The term “domain” refers to a structure of a biomolecule that contributes to a known or suspected function of the biomolecule. Domains may be co-extensive with regions or portions thereof, domains may also include distinct, non-contiguous regions of a biomolecule. Examples of GP354 protein domains include, but are not limited to, an extracellular Ig domain (i.e., N-terminal), a transmembrane domain, and a cytoplasmic domain (i.e., C-terminal).

As used herein, the term “compound” means any molecule, including, but not limited to, small molecule, peptide, protein, sugar, nucleotide, nucleic acid, lipid, etc., and such a compound can be natural or synthetic.

Unless otherwise defined, all technical and scientific terms used herein have the same meaning as commonly understood by one of ordinary skill in the art to which this invention pertains. Exemplary methods and materials are described below, although methods and materials similar or equivalent to those described herein can also be used in the practice of the present invention and will be apparent to those of skill in the art. All publications and other references mentioned herein are incorporated by reference in their entirety. In case of conflict, the present specification, including definitions, will control. The materials, methods, and examples are illustrative only and not intended to be limiting.

Standard reference works setting forth the general principles of recombinant DNA technology known to those of skill in the art include Ausubel et al., CURRENT PROTOCOLS IN MOLECULAR BIOLOGY, John Wiley & Sons, New York (1998 and Supplements to 2001); Sambrook et al., MOLECULAR CLONING: A LABORATORY MANUAL, 2d Ed., Cold Spring Harbor Laboratory Press, Plainview, N.Y. (1989); Kaufman et al., Eds., HANDBOOK OF MOLECULAR AND CELLULAR METHODS IN BIOLOGY AND MEDICINE, CRC Press, Boca Raton (1995); McPherson, Ed., DIRECTED MUTAGENESIS: A PRACTICAL APPROACH, IRL Press, Oxford (1991). Standard reference works setting forth the general principles of immunology known to those of skill in the art include: Harlow and Lane ANTIBODIES: A LABORATORY MANUAL, 2d Ed., Cold Spring Harbor Laboratory Press, Cold Spring Harbor, N.Y. (1999); and Roitt et al., IMMUNOLOGY, 3d Ed., Mosby-Year Book Europe Limited, London (1993). Standard reference works setting forth the general principles of medical physiology and pharmacology known to those of skill in the art include: Harrison's PRINCIPLES OF INTERNAL MEDICINE, 14^{th} Ed., (Anthony S. Fauci et al., editors), McGraw-Hill Companies, Inc., 1998.

### GP354 Related Nucleic Acids

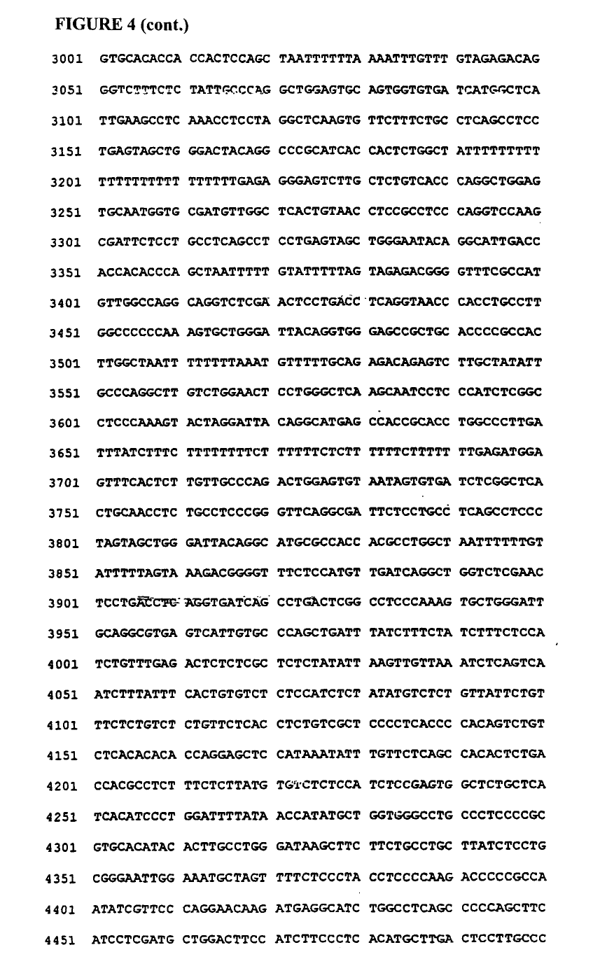

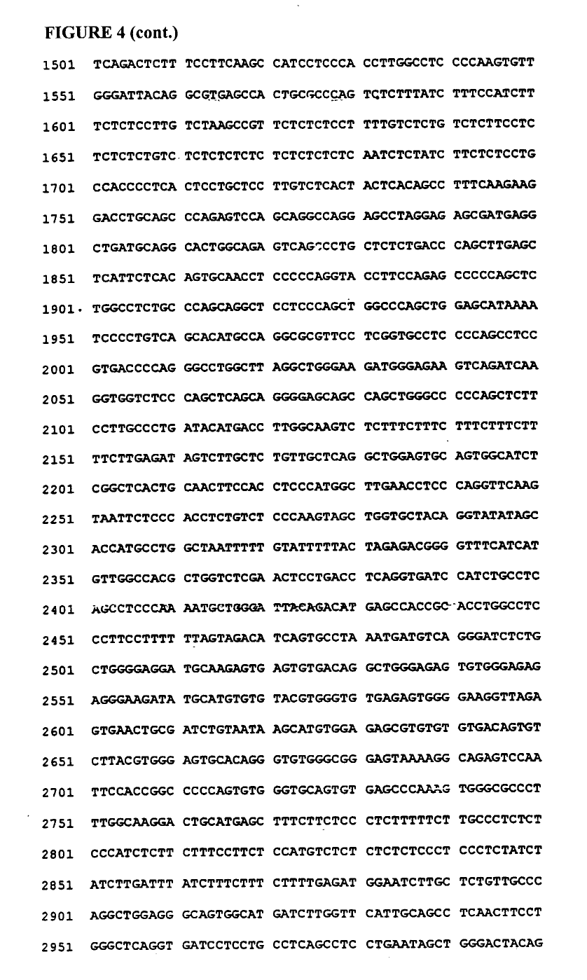

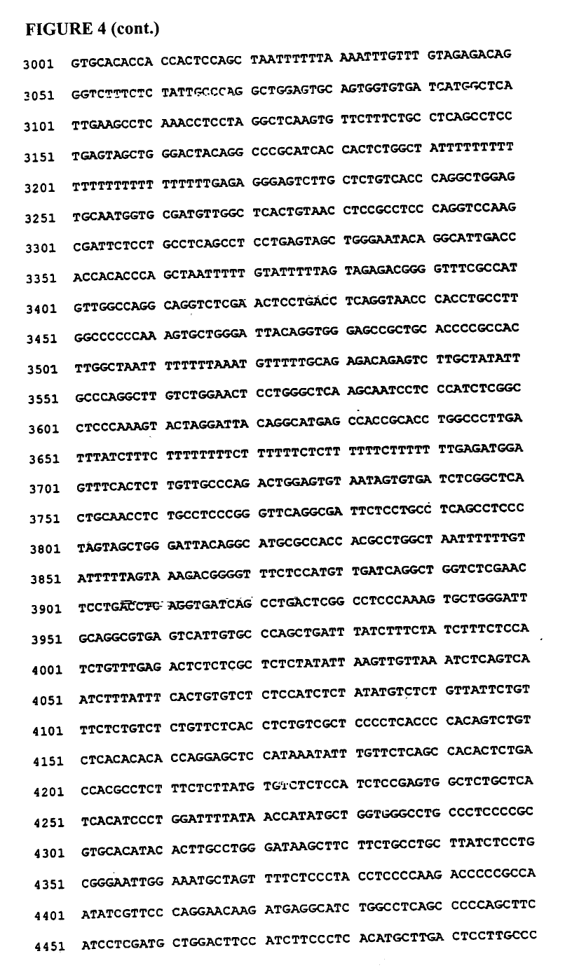

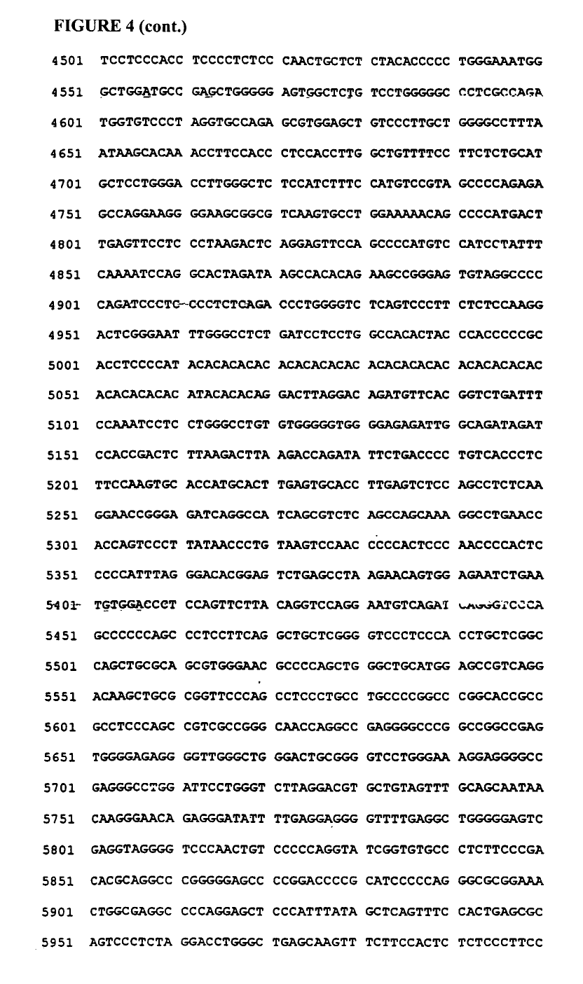

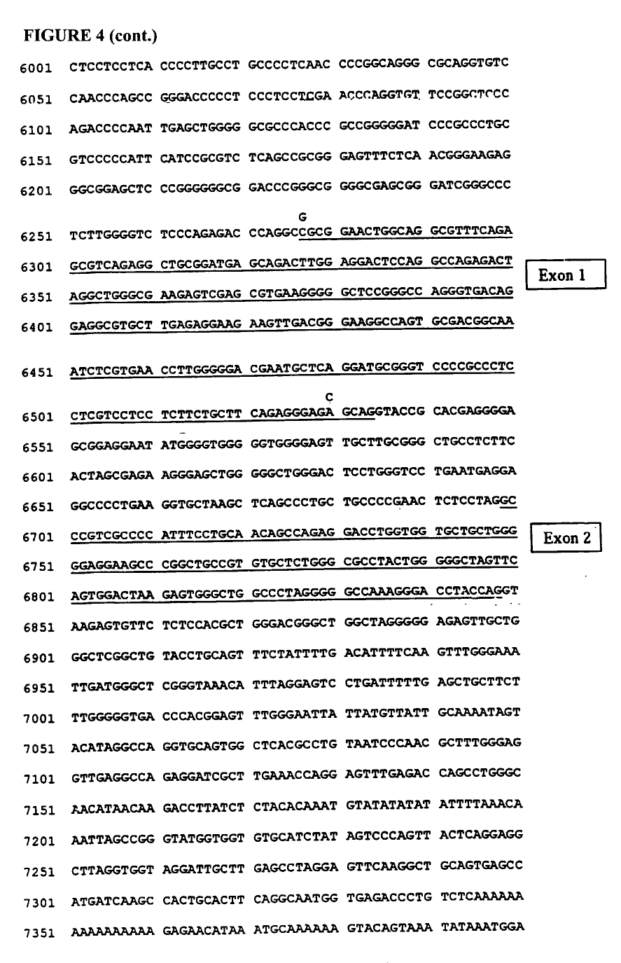

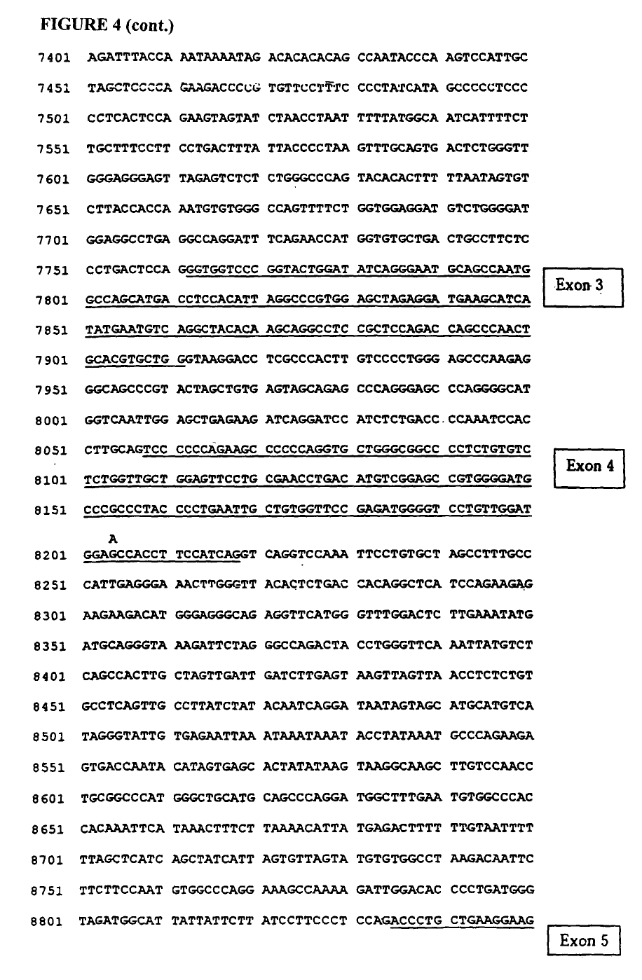

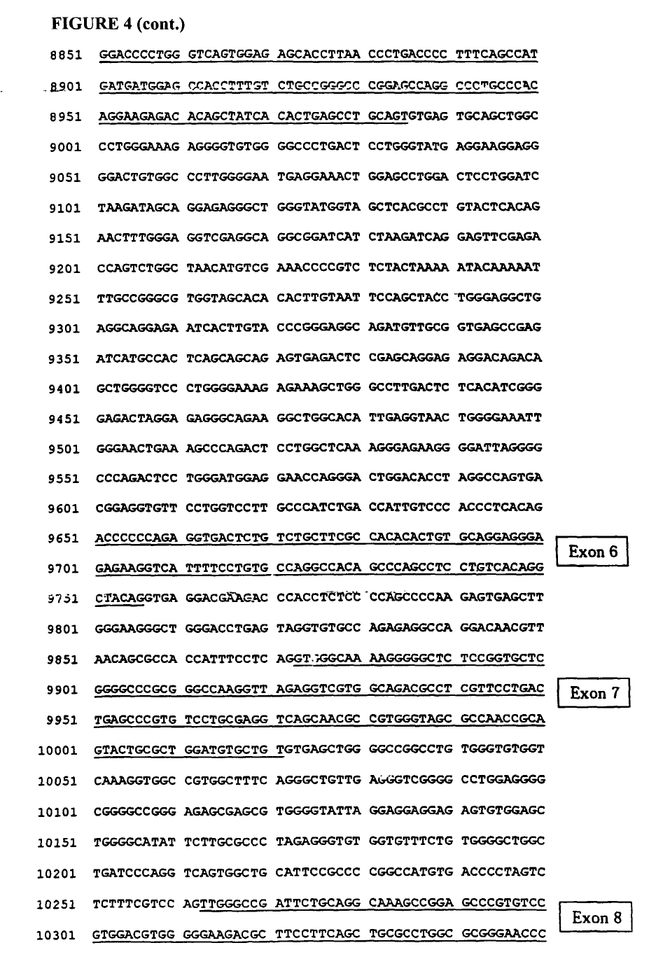

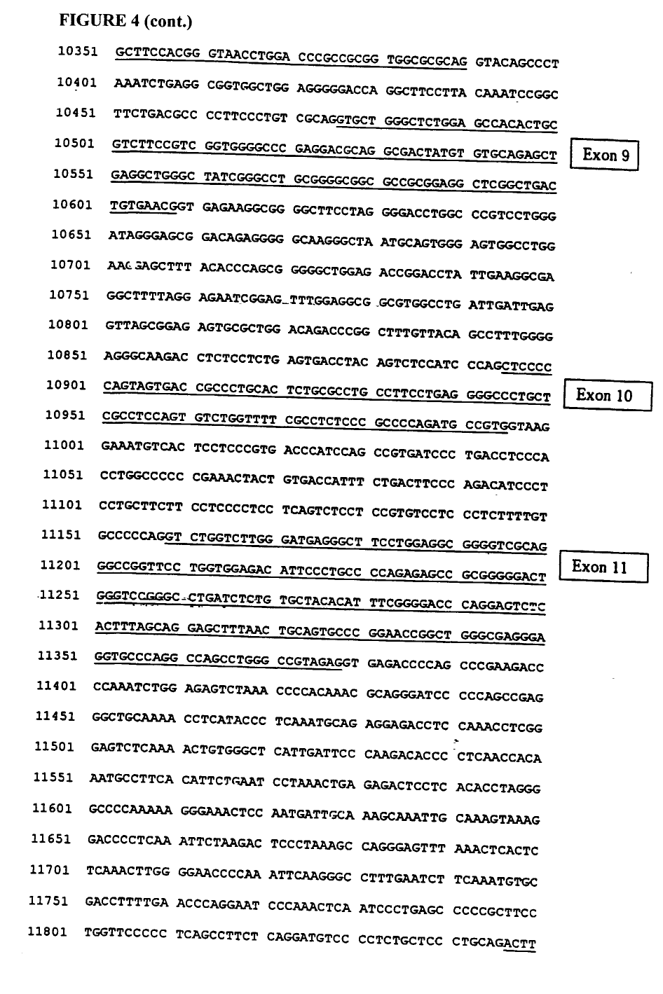

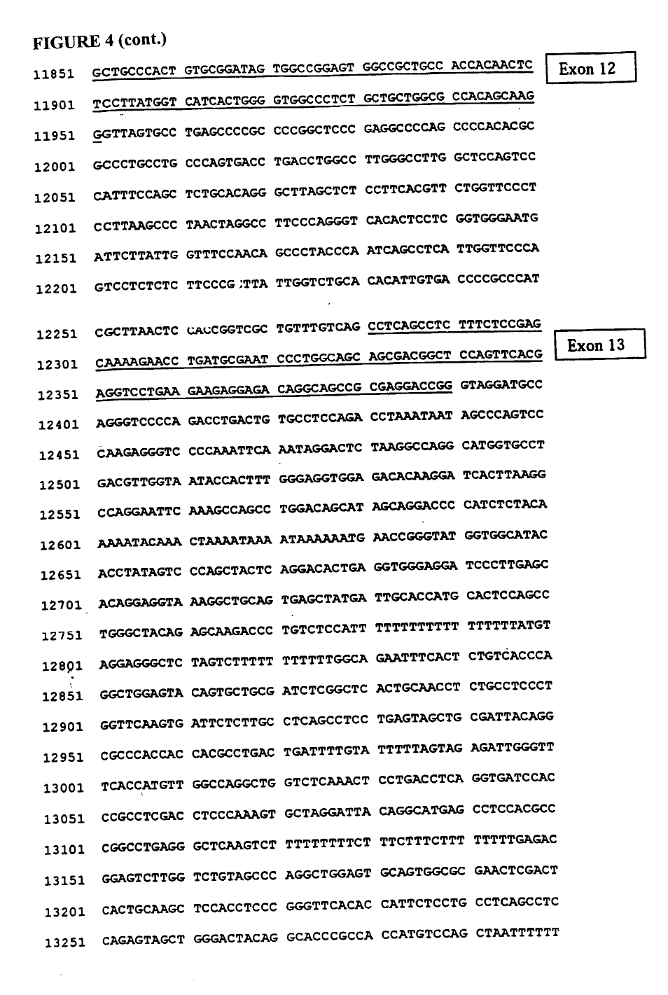

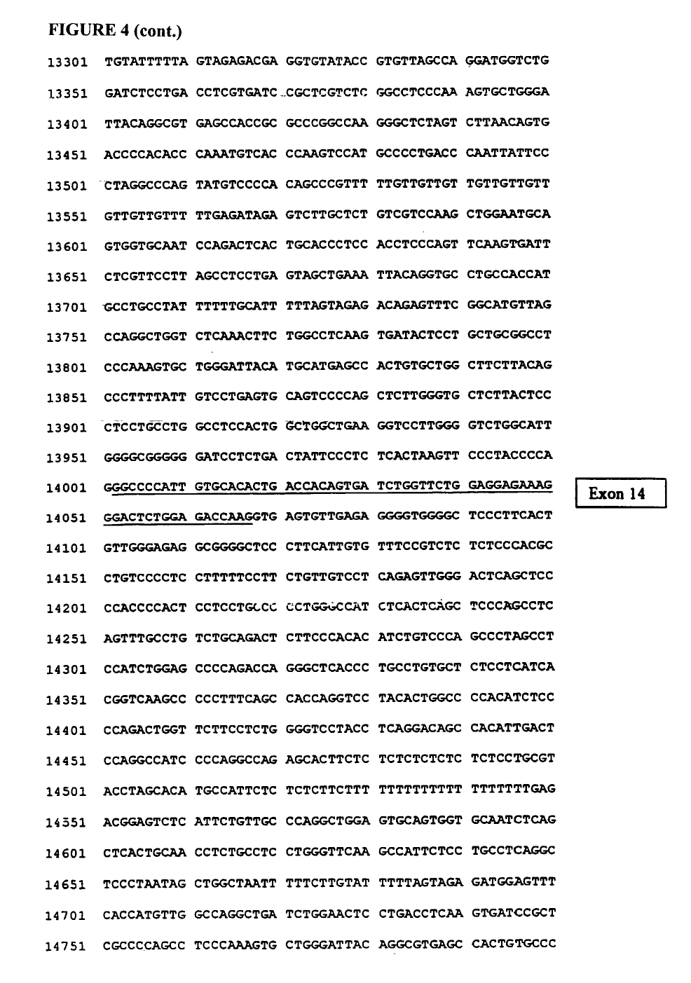

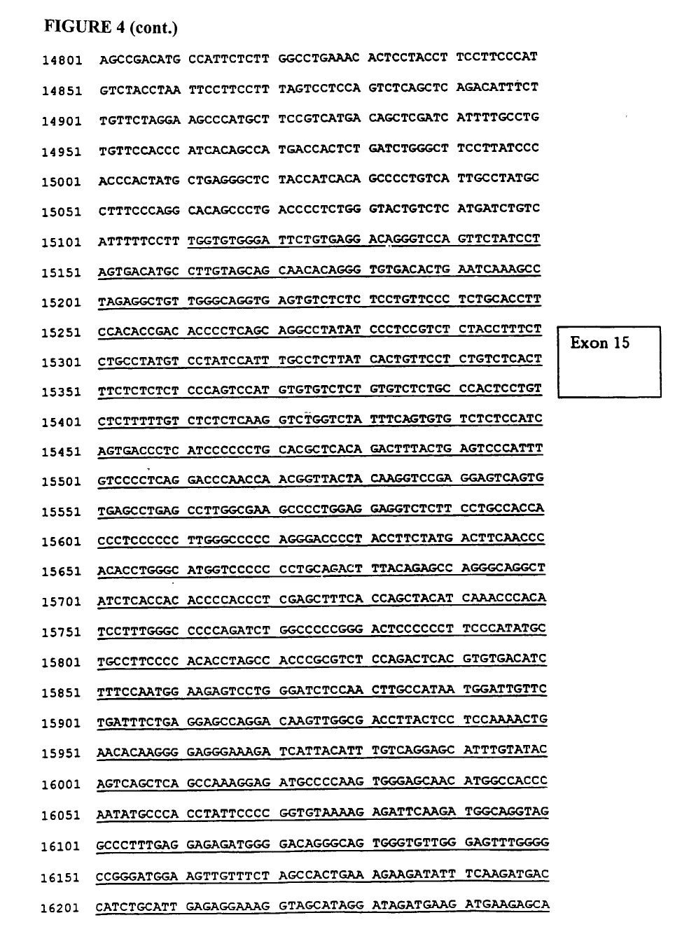

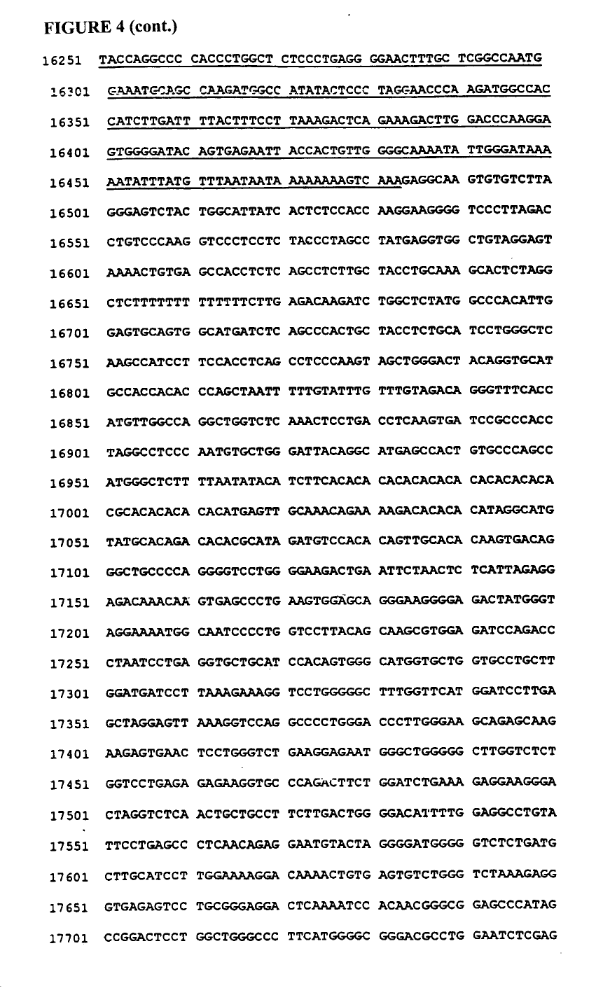

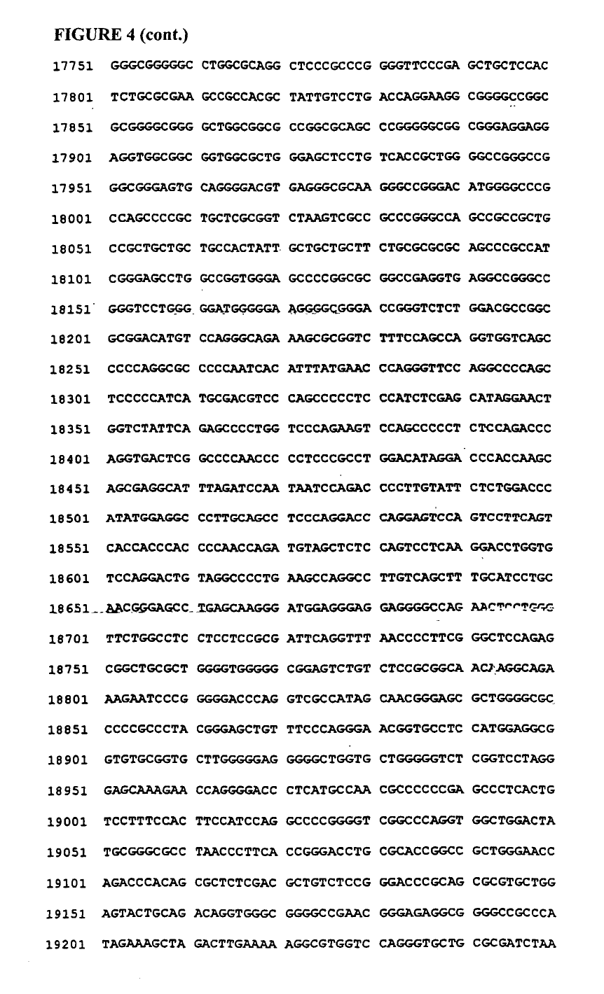

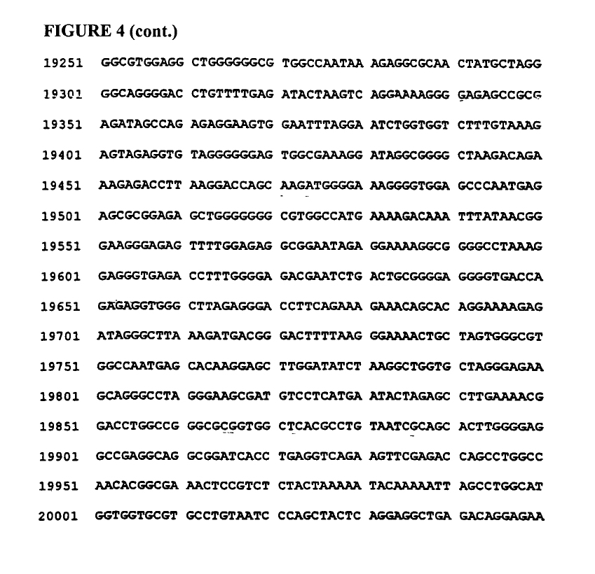

The gp354 gene was identified in contig 38 of a BAC clone with the GenBank accession number AC022315, which was deposited on February 10, 2000. That deposit has the human genomic sequence of gp354 (FIG. 4 and SEQ ID NO:5), including 5′ upstream (positions 1-6278) and 3′ downstream (16490-20050) non-transcribed genomic sequences.

The invention provides isolated polynucleotides that encode the entirety of the GP354 protein. As discussed above, such “full-length” polynucleotides of the present invention can be used, inter alia, to express full length GP354 protein. The full-length polynucleotides can also be used as nucleic acid probes; used as probes, the isolated polynucleotides of these embodiments will hybridize to gp354 polynucleotides and related polynucleotide sequences.

In preferred embodiments, the invention provides an isolated polynucleotide comprising part or all of (i) the nucleotide sequence of SEQ ID NO:1, 3, 5, 6, 7, 9, 11, 25, 27, or 32; (ii) a degenerate variant of thereof; or (iii) the complement of (i) or (ii).

SEQ ID NO:1 presents a predicted gp354 cDNA sequence. SEQ ID NO:3 presents an exemplary gp354 cDNA fragment generated from RT-PCR. SEQ ID NO:5 presents the genomic DNA sequence comprising the gp354 coding sequences, including 5′ and 3′ non-transcribed regions. SEQ ID NO:6 presents the genomic DNA sequence comprising the upstream noncoding region. SEQ ID NO:7 presents a derived, long form of a gp354 cDNA sequence. SEQ ID NO:11 presents another exemplary pancreatic gp354 cDNA sequence. SEQ ID NOs:25 and 27 present the coding sequence of PanCAM-1 and PanCAM-2, respectively, both being alternative splice variants of SEQ ID NO:7.

In other embodiments, the invention provides an isolated polynucleotide comprising (i) a nucleotide sequence that encodes a polypeptide with the amino acid sequence of SEQ ID NO:2, 4, 8, 10, 12, 26, 28 or 31; or (ii) the complement of a nucleotide sequence that encodes the above polypeptide.

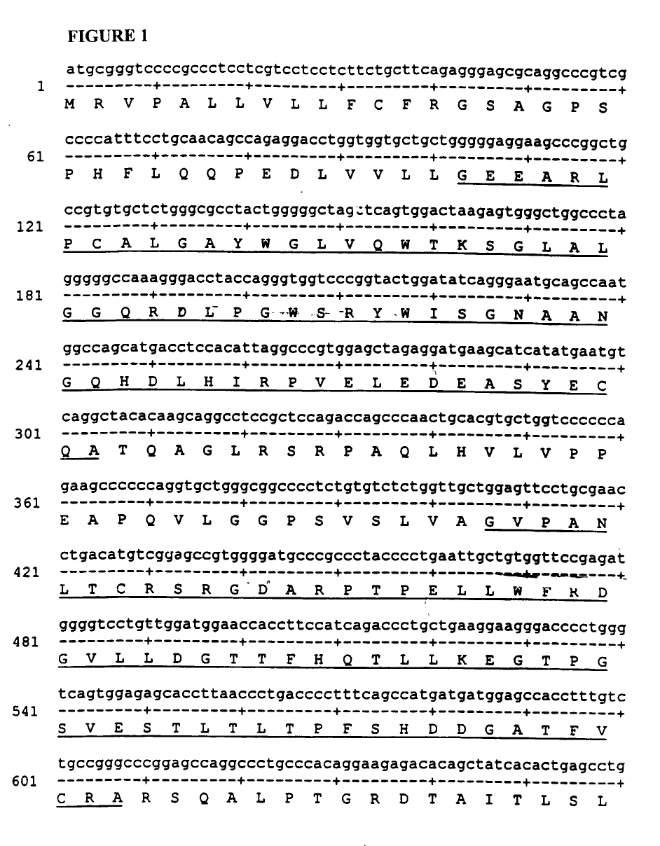

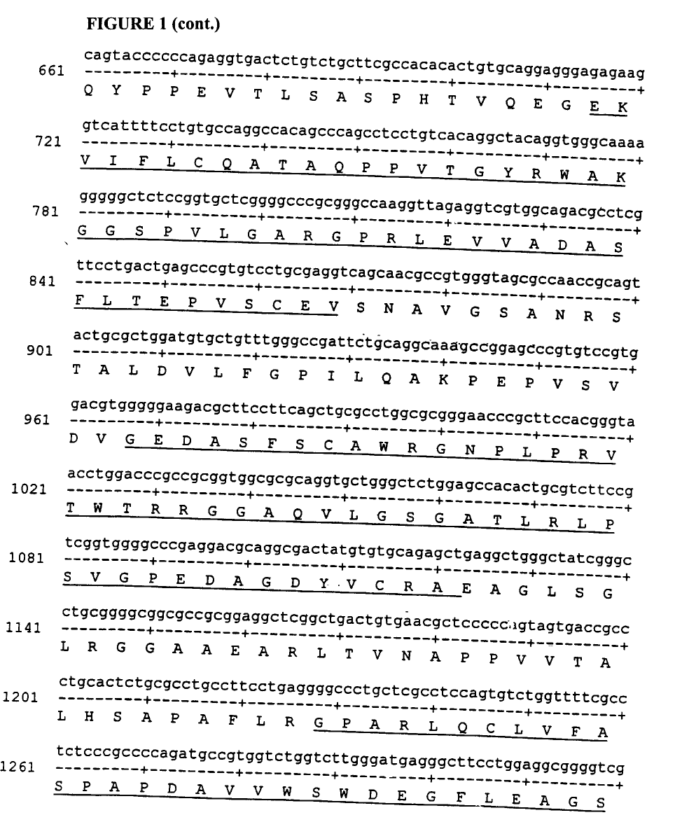

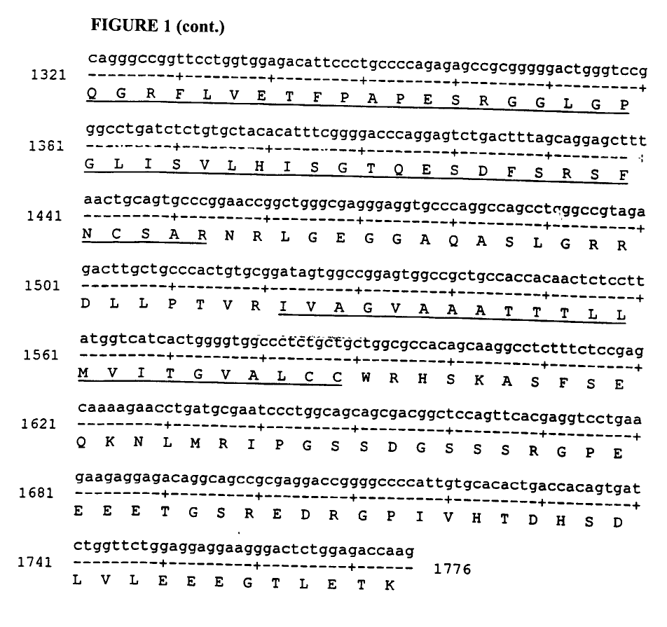

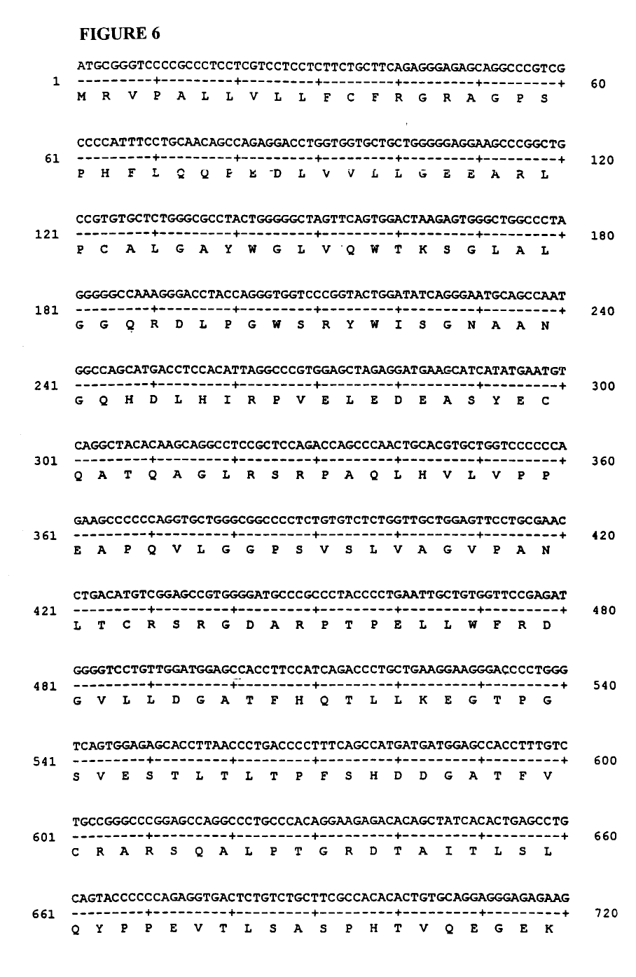

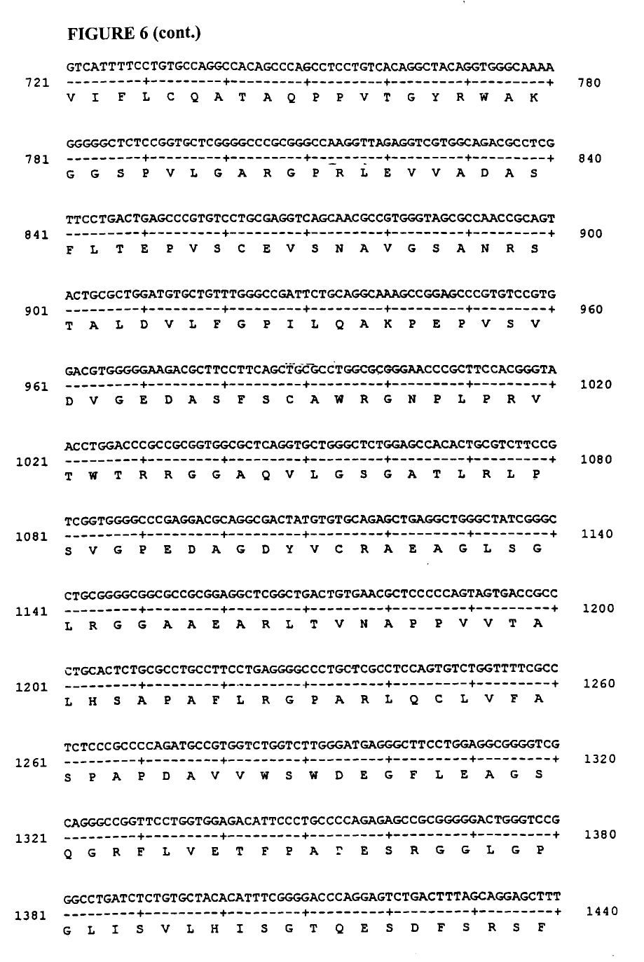

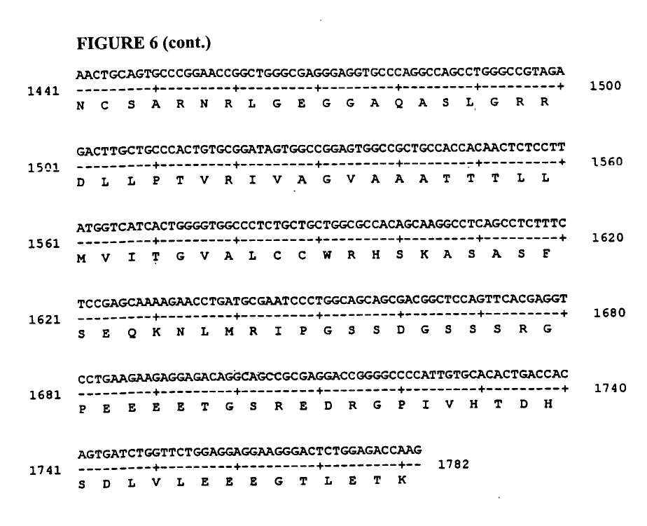

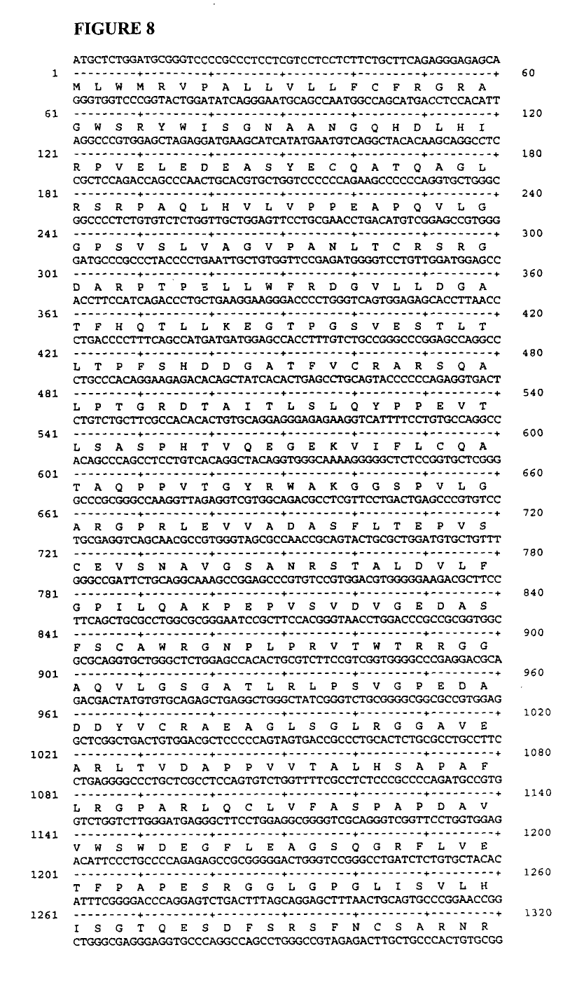

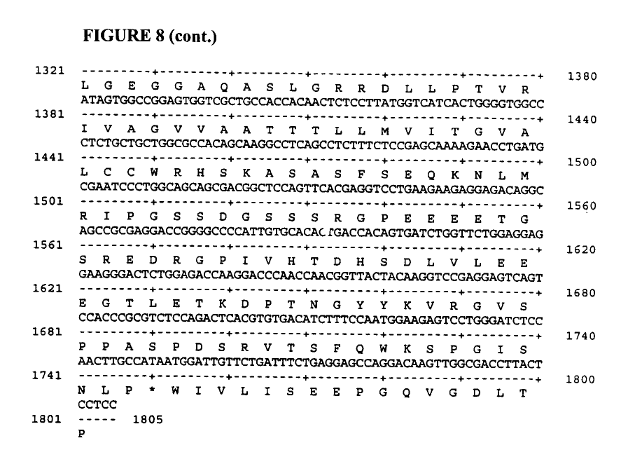

SEQ ID NOs:2, 4, 8, 10, 12, 26, 28 and 31 present the GP354 amino acid sequences encoded by SEQ ID NOs:1, 3, 7, 9, 11, 25, 27 and 32, respectively. See, e.g., FIGS. 1-10.

In other embodiments, the invention provides an isolated polynucleotide having a nucleotide sequence that (i) encodes a polypeptide having the sequence of SEQ ID NO:2, 8 or 12, (ii) encodes a polypeptide having the sequence of SEQ ID NO:2, 8 or 12 with conservative amino acid substitutions, or (iii) that is the complement of (i) or (ii), where SEQ ID NO:2 presents the amino acid sequence of GP354 encoded by the cDNA of SEQ ID NO:1; SEQ ID NO:8 presents the amino acid sequence of GP354 encoded by sequences derived from SEQ ID NOs:5 and 11; and SEQ ID NO:12 presents the amino acid sequence of GP354 encoded by the pancreatic cDNA of SEQ ID NO:11.

### Nucleic Acids Encoding Portions Of GP354

The invention also provides isolated polynucleotides that encode select portions of GP354. As will be further discussed herein below, these “nucleic acid molecules” can be used, for example, to express specific portions of the GP354, either alone or as elements of a fusion protein. A nucleic acid fragment may also be used as a region-specific nucleic acid probe.

In preferred embodiments, the invention provides an isolated polynucleotide comprising (i) the nucleotide sequence of SEQ ID NO:3, 6 or 9, (ii) a degenerate variant of the nucleotide sequence of SEQ ID NO:3, 6 or 9, or (iii) the complement of (i) or (ii). SEQ ID NO:3 presents a 785 base pair RT-PCR fragment derived from gp354 pancreatic RNA. SEQ ID NO:6 presents genomic sequences upstream from gp354 coding sequences, and SEQ ID NO:9 presents a 1782 base pair RT-PCR fragment derived from gp354 pancreatic RNA.

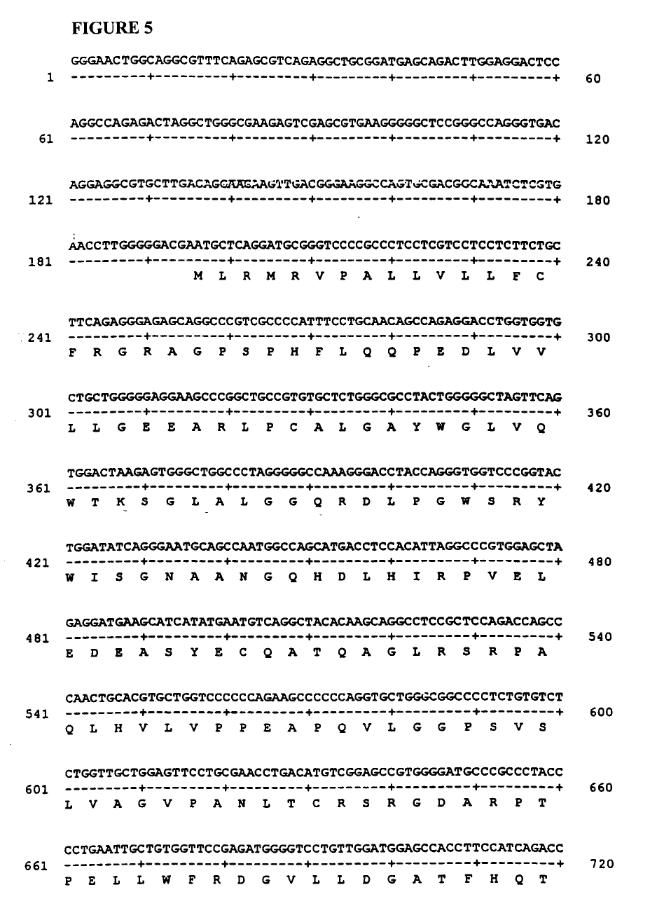

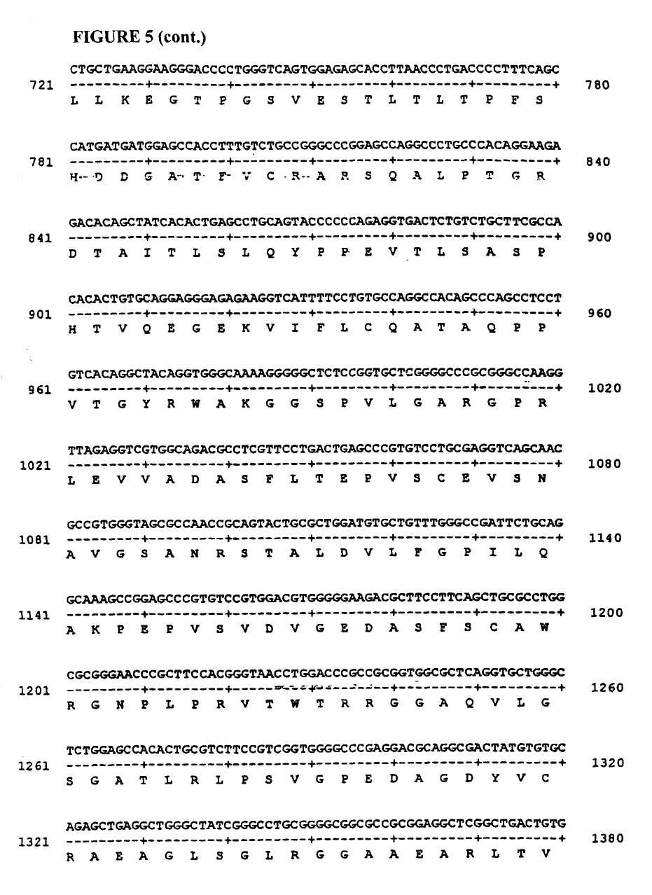

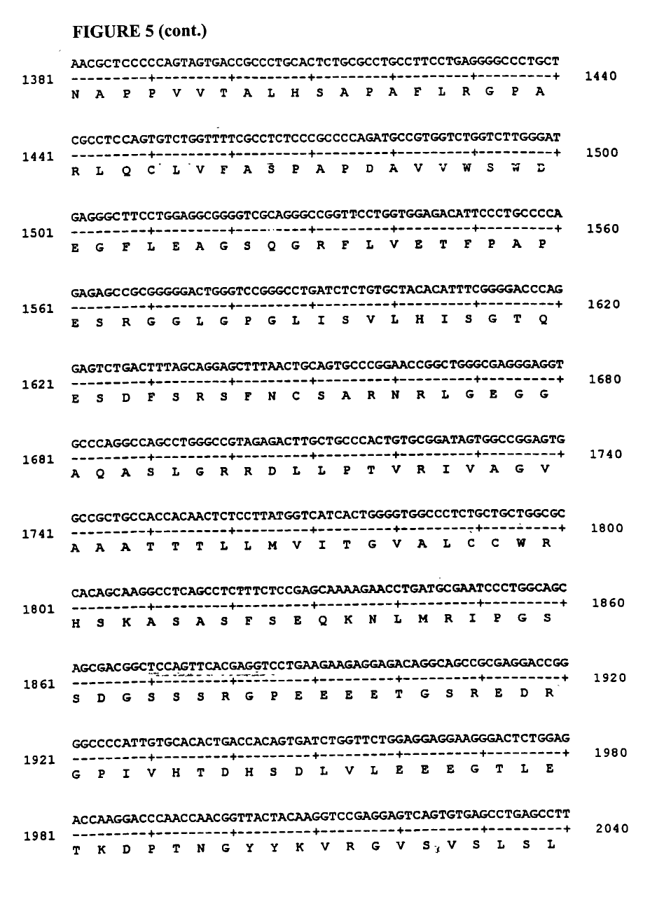

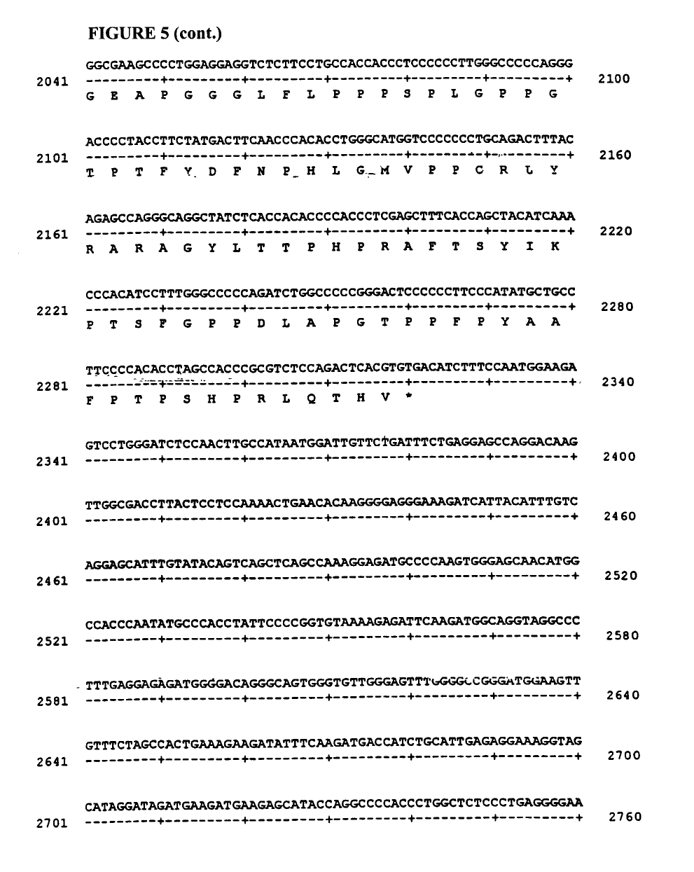

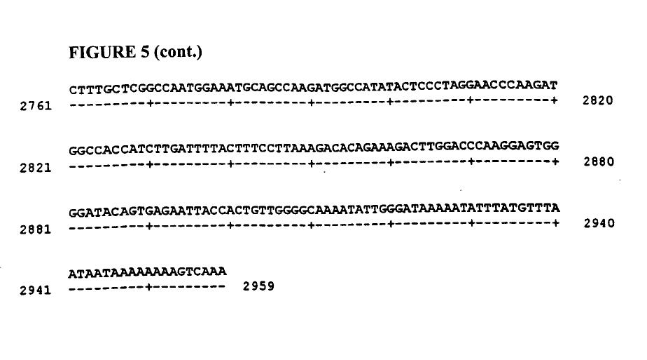

In other embodiments, the isolated polynucleotide encodes, or the complement of which encodes, a polypeptide having, in at least one and preferably two, three, four or five of the Ig domains characteristic of the N-terminal extracellular portion of GP354. Specifically, the five extracellular Ig domains are encoded by nucleotides 103-306, 406-609, 715-870, 967-1122 and 1228-1445, respectively, of the gp354 cDNA sequence of SEQ ID NO:1 (see FIG. 1) and by nucleotides 307-510, 610-813, 919-1074, 1171-1326 and 1432-1659, respectively, of the gp354 cDNA sequence of SEQ ID NO:8 (see FIG. 5). In preferred embodiments, the isolated polynucleotide encodes at least two, preferably three, more preferably four and most preferably all five domains in at least one copy.

For some uses, such as protein production, the nucleic acid fragments (or their complements) comprise sequences which encode a signal secretion sequence that will mediate transport of the encoded polypeptides through a membrane. Such is signal sequence is typically cleaved from the polypeptides as transport through the membrane occurs. The GP354 signal secretion sequence is encoded by nucleotides 1-54 of the gp354 cDNA sequence of SEQ ID NO:1 (see FIG. 1) and by nucleotides 1-57 of the gp354 cDNA of SEQ ID NO:8 (see FIG. 5). More preferably, the signal secretion sequence of the isolated polynucleotide of the invention is from gp354. Assuming that the signal sequence of GP354 is also cleaved during secretion, the mature GP354 polypeptide sequence has an N-terminal proline residue encoded by nucleotides 55-57 of SEQ ID NO:1 (see FIG. 1) and by nucleotides 259-261 of the gp354 cDNA of SEQ ID NO:8 (see FIG. 5).

Other preferred embodiments of the polynucleotides of the invention are those that encode, or the complements of which encode, a polypeptide having the transmembrane domain of GP354. The above preferred isolated polynucleotides, for example, may optionally encode a transmembrane domain, if insertion of the encoded polypeptides into a membrane is so-desired. The transmembrane domain may be encoded by gp354 sequences or may be encoded by a heterologous gene encoding a transmembrane domain of a heterologous membrane-associated protein. The gp354 transmembrane domain is encoded by nucleotides 1522-1590 of the gp354 cDNA sequence of SEQ ID NO:1 (see FIG. 1) and by nucleotides 1726-1794 of the gp354 cDNA of SEQ ID NO:8 (see FIG. 5).

If so-desired, the isolated polynucleotides of the invention may comprise sequences which encode (or their complements encode) an intracellular C-terminal domain, e.g., if specific signaling reactions are desired in response to GP354 binding interactions. The intracellular domain may be encoded by gp354 (see below) or may be encoded by a heterologous gene encoding an intracellular domain of a heterologous membrane-associated protein. Preferred polynucleotides of the invention are those that encode, or the complements of which encode, a polypeptide having a (C-terminal) intracellular domain of GP354. Specifically, one intracellular domain of GP354 is encoded by nucleotides 1591-1776 of the gp354 cDNA sequence of SEQ ID NO:1 (see FIG. 1). A longer form of an intracellular domain of GP354 is encoded by nucleotides 1795-2319 of the gp354 cDNA sequence of SEQ ID NO:8 (see FIG. 5).

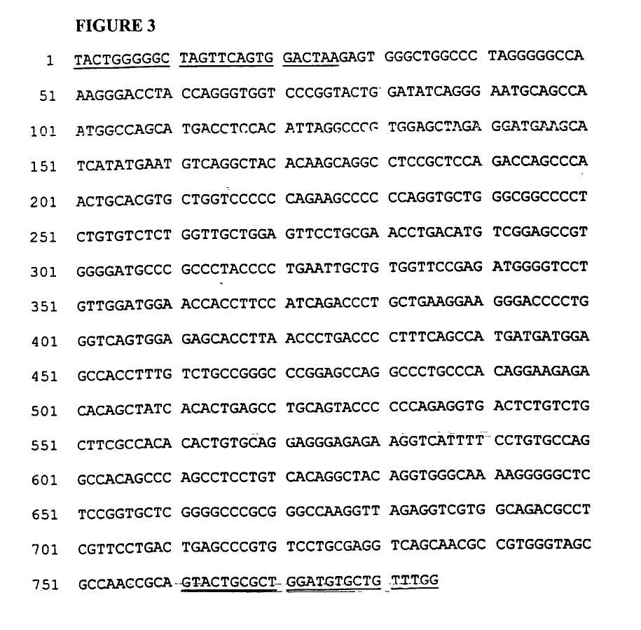

One preferred isolated polynucleotide of the invention is shown in FIG. 3 (see SEQ ID NO:3) and comprises nucleotides 139-923 of the gp354 cDNA sequence of SEQ ID NO:1 (see FIG. 1). It comprises the sequence of an RT-PCR fragment amplified from pancreatic RNA using primers GX1-218 (SEQ ID NO:16) and GX1-219 (SEQ ID NO:17). See Example 2. This preferred isolated polynucleotide encodes amino acids 47-307 of SEQ ID NO:2, i.e., it encodes amino acids 13-68 of the first N-terminal Ig domain (i.e., it is missing the first 12 N-terminal amino acids of the Ig domain), and encodes the second and third Ig domains of GP354.

### Cross-Hybridizing Nucleic Acids

In another series of nucleic acid embodiments, the invention provides isolated polynucleotides that hybridize to various of the gp354 nucleic acids of the present invention. These “cross-hybridizing nucleic acids” can be used, inter alia, as probes for, and to drive expression of, proteins that are related to gp354 of the present invention as further isoforms, homologs, paralogs, or orthologs.

In some such embodiments, the invention provides an isolated polynucleotide comprising a sequence that hybridizes under high stringency conditions to a probe the nucleotide sequence of which comprises SEQ ID NO:1, 5, 7, 9, or 11; the complement of SEQ ID NO:1, 5, 7, 9, or 11; or a fragment thereof having at least 17 nucleic acid units.

### Preferred Nucleic Acids

Particularly preferred among the above-described nucleic acids are those that are expressed, or the complements of which are expressed, in pancreatic or neural tissues. Also particularly preferred among the above-described nucleic acids are those that encode, or the complements of which encode, a polypeptide having a gp354 biological activity, as described supra.

### Nucleic Acid Fragments

In another series of nucleic acid embodiments, the invention provides fragments of various of the isolated polynucleotides of the present invention which prove useful, inter alia, as region-specific nucleic acid probes, as amplification primers, and to direct expression or synthesis of epitopic or immunogenic protein fragments.

In some embodiments, the invention provides an isolated polynucleotide comprising at least 17 nucleotides, 18 nucleotides, 20 nucleotides, 24 nucleotides, or 25 nucleotides of contiguous nucleic acid sequence selected from SEQ ID NO:1, 5, 7, 9, or 11.

In other embodiments, the invention provides an isolated nucleic acid comprising a nucleotide sequence that (i) encodes a polypeptide having the sequence of at least eight contiguous amino acids of SEQ ID NO:2, 4, 8, 10 or 12 (ii) encodes a polypeptide having the sequence of at least eight contiguous amino acids of SEQ ID NO:2, 4, 8, 10 or 12 with conservative amino acid substitutions, or (iii) is the complement of (i) or (ii).

### Single Exon Probes

The invention further provides genome-derived single exon probes having portions of no more than one exon of the gp354 gene. Such single exon probes have particular utility in identifying and characterizing splice variants. In particular, such single exon probes are useful for identifying and discriminating the expression of distinct isoforms of gp354.

In some embodiments, the invention provides an isolated nucleic acid comprising a nucleotide sequence selected from one of the following exon-specific portions of SEQ ID NO:1, 5, 7, 9, or 11 or the complement of SEQ ID NO:1, 5, 7, 9, or 11, wherein the portion comprises at least 17 contiguous nucleotides, 18 contiguous nucleotides, 20 contiguous nucleotides, 24 contiguous nucleotides, 25 contiguous nucleotides, or 50 contiguous nucleotides of any one of the portions of SEQ ID NO:1, 5, 7, 9, or 11, or their complement:

TABLE 1
Exon coordinates of gp354 cDNA (SEQ ID NO: 1 or 2) and genomic
(SEQ ID NO: 5) sequences

| | cDNA-1 | cDNA-2 | genomic |
|---|---|---|---|
| exon 1 | 1-52 | 1-52 | 6483-6534 |
| exon 2 | 53-202 | 53-202 | 6699-6848 |
| exon 3 | 203-352 | 203-352 | 7762-7911 |
| exon 4 | 353-513 | 353-513 | 8058-8218 |
| exon 5 | 514-664 | 514-664 | 8835-8985 |
| exon 6 | 665-770 | 665-770 | 9651-9756 |
| exon 7 | 771-919 | 771-919 | 9873-10021 |
| exon 8 | 920-1047 | 920-1041 | 10263-10390 |
| exon 9 | 1048-1180 | 1042-1180 | 10476-10608 |
| exon 10 | 1181-1281 | 1181-1281 | 10895-10995 |
| exon 11 | 1282-1501 | 1282-1501 | 11159-11378 |
| exon 12 | 1502-1606 | 1502-1606 | 11847-11951 |
| exon 13 | 1607-1710 | 1607-1716 | 12287-12390 |
| exon 14 | 1711-1779 | 1717-1782 | 14002-14067 |

TABLE 2
Exon coordinates of gp354 cDNA-4 (SEQ ID NO: 11) and genomic
(SEQ ID NO: 5) sequences

| | cDNA | genomic |
|---|---|---|
| Exon 1 | 1-256 | 6278-6534 |
| Exon 2 | 257-406 | 6699-6848 |
| Exon 3 | 407-556 | 7762-7911 |
| Exon 4 | 557-717 | 8058-8218 |
| Exon 5 | 718-868 | 8835-8985 |
| Exon 6 | 869-974 | 9651-9756 |
| Exon 7 | 975-1123 | 9873-10021 |
| Exon 8 | 1124-1245 | 10263-10390 |
| Exon 9 | 1246-1384 | 10476-10608 |
| Exon 10 | 1385-1485 | 10895-10995 |
| Exon 11 | 1486-1705 | 11159-11378 |
| Exon 12 | 1706-1810 | 11847-11951 |
| Exon 13 | 1811-1920 | 12281-12390 |
| Exon 14 | 1921-1986 | 14002-14067 |
| Exon 15 | 1987-2959 | 15511-16483 |

### Transcription Control Nucleic Acids

In another aspect, the present invention provides genome-derived isolated polynucleotides which include nucleic acid sequence elements that control transcription of the gp354 gene. These nucleic acids can be used, inter alia, to drive expression of heterologous coding regions in recombinant constructs, thus conferring upon such heterologous coding regions the expression pattern of the native gp354 gene. These nucleic acids can also be used, conversely, to target heterologous transcription control elements to the gp354 genomic locus, altering the expression pattern of the gp354 gene itself.

In a first series of such embodiments, the invention provides an isolated polynucleotide comprising nucleotides 1-6483 of SEQ ID NO:5; nucleotides 1483-6482 of SEQ ID NO:5; nucleotides 2483-6482 of SEQ ID NO:5; nucleotides 3483-6482 of SEQ ID NO:5; nucleotides 4483-6482 of SEQ ID NO:5; nucleotides 5483-6482 of SEQ ID NO:5; or nucleotides 5983-6482 of SEQ ID NO:5; or the complements of such sequences.

In other embodiments, the invention provides an isolated polynucleotide comprising at least 17, 18, 20, 24, or 25 nucleotides of nucleotides 1-6483 of SEQ ID NO:5; nucleotides 1483-6482 of SEQ ID NO:5; nucleotides 2483-6482 of SEQ ID NO:5; nucleotides 3483-6482 of SEQ ID NO:5; nucleotides 4483-6482 of SEQ ID NO:5; nucleotides 5483-6482 of SEQ ID NO:5; or nucleotides 5983-6482 of SEQ ID NO:5; or the complements of such sequences.

Each of the isolated polynucleotides comprising nucleotides 1-6483 of SEQ ID NO:5; nucleotides 1483-6482 of SEQ ID NO:5; nucleotides 2483-6482 of SEQ ID NO:5; nucleotides 3483-6482 of SEQ ID NO:5; nucleotides 4483-6482 of SEQ ID NO:5; nucleotides 5483-6482 of SEQ ID NO:5; or nucleotides 5983-6482 of SEQ ID NO:5; or the complements of such sequences has transcription control sequences that mediate developmental and tissue specific expression and regulation of the gp354 gene. Such transcription control sequences will be useful for conferring such developmental and tissue specific expression patterns on heterologous nucleic acid sequences operatively linked thereto.

### Other Defining Features of gp354 Nucleic Acid Molecules

All the nucleic acid sequences specifically given herein are set forth as sequences of deoxyribonucleotides. It is intended, however, that the given sequences be interpreted as would be appropriate to the polynucleotide composition: for example, if the isolated nucleic acid is composed of RNA, the given sequence intends ribonucleotides, with uridine substituted for thymidine.

Polymorphisms such as single nucleotide polymorphisms (SNPs) occur frequently in eukaryotic genomes. More than 1.4 million SNPs have already identified in the human genome, International Human Genome Sequencing Consortium, Nature 409:860-921 (2001)—and the sequence determined from one individual of a species may differ from other allelic forms present within the population. Additionally, small deletions and insertions, rather than single nucleotide polymorphisms, are not uncommon in the general population, and often do not alter the function of the protein.

Accordingly, it is particularly emphasized that the present invention not only provides isolated polynucleotides identical in sequence to those described with particularity herein (e.g., SEQ ID NOs:1, 3, 5, 6, 7, 9 and 11), but also to provide isolated polynucleotides that are allelic variants of those particularly described nucleic acid sequences. Further, the invention provides homologs (e.g., paralogs and orthologs) of gp354 that are at least about 65% identical in sequence to SEQ ID NOs:1, 3, 5, 6, 7, 9 and 11, or to a portion of any one of those sequences that encodes at least one Ig domain, typically at least about 70%, 75%, 80%, 85%, or 90% identical in sequence, usefully at least about 91%, 92%, 93%, 94%, or 95% identical in sequence, more usefully at least about 96%, 97%, 98%, or 99% identical in sequence, and, most conservatively, at least about 99.5%, 99.6%, 99.7%, 99.8% and 99.9% identical in sequence to those described with particularity herein. These sequence variants can be naturally occurring or can result from human intervention, as by random or directed mutagenesis.

Nucleic acid sequence variants have been found to occur, e.g., at positions 252, 703, 770, 1249 and 1811-1816 of the sequence presented in SEQ ID NO:7.

For purposes herein, percent identity of two nucleic acid sequences is determined using the procedure of Tatiana et al., “Blast 2 sequences—a new tool for comparing protein and nucleotide sequences”, FEMS Microbiol Lett. 174:247-250 (1999), which procedure is effectuated by the computer program BLAST 2 SEQUENCES, available online at the National Center for Biotechnology Information.

To assess percent identity of nucleic acid sequences, the BLASTN module of BLAST 2 SEQUENCES is used with default values of (i) reward for a match: 1; (ii) penalty for a mismatch: −2; (iii) open gap 5 and extension gap 2 penalties; (iv) gap X_dropoff 50 expect 10 word size 11 filter, and both sequences are entered in their entireties.

The isolated polynucleotides of the present invention being useful for expression of GP354 proteins and protein fragments, the present invention thus provide isolated polynucleotides that encode GP354 proteins and portions thereof not only identical in sequence to those described with particularity herein, but degenerate variants thereof as well. As is well known, the genetic code is degenerate and codon choice for optimal expression varies from species to species.

As is also well known, amino acid substitutions occur frequently among natural allelic variants, with conservative substitutions often occasioning only de minimis change in protein function.

Accordingly, the present invention provides polynucleotides not only identical in sequence to those described with particularity herein, but also those that encode GP354 and portions thereof, having conservative amino acid substitutions or moderately conservative amino acid substitutions.

Although there are a variety of metrics for calling conservative amino acid substitutions, based primarily on either observed changes among evolutionarily related proteins or on predicted chemical similarity, for purposes herein a conservative replacement is any change having a positive value in the PAM250 log-likelihood matrix reproduced herein below (see Gonnet et al., Science 256(5062):1443-5 (1992)):

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 2 | −1 | 0 | 0 | 0 | 0 | 0 | 0 | −1 | −1 | −1 | 0 | −1 | −2 | 0 | 1 | 1 | −4 | −2 | 0 |
| R | −1 | 5 | 0 | 0 | −2 | 2 | 0 | −1 | 1 | −2 | −2 | 3 | −2 | −3 | −1 | 0 | 0 | −2 | −2 | −2 |
| N | 0 | 0 | 4 | 2 | −2 | 1 | 1 | 0 | 1 | −3 | −3 | 1 | −2 | −3 | −1 | 1 | 0 | −4 | −1 | −2 |
| D | 0 | 0 | 2 | 5 | −3 | 1 | 3 | 0 | 0 | −4 | −4 | 0 | −3 | −4 | −1 | 0 | 0 | −5 | −3 | −3 |
| C | 0 | −2 | −2 | −3 | 12 | −2 | −3 | −2 | −1 | −1 | −2 | −3 | −1 | −1 | −3 | 0 | 0 | −1 | 0 | 0 |
| Q | 0 | 2 | 1 | 1 | −2 | 3 | 2 | −1 | 1 | −2 | −2 | 2 | −1 | −3 | 0 | 0 | 0 | −3 | −2 | −2 |
| E | 0 | 0 | 1 | 3 | −3 | 2 | 4 | −1 | 0 | −3 | −3 | 1 | −2 | −4 | 0 | 0 | 0 | −4 | −3 | −2 |
| G | 0 | −1 | 0 | 0 | −2 | −1 | −1 | 7 | −1 | −4 | −4 | −1 | −4 | −5 | −2 | 0 | −1 | −4 | −4 | −3 |
| H | −1 | 1 | 1 | 0 | −1 | 1 | 0 | −1 | 6 | −2 | −2 | 1 | −1 | 0 | −1 | 0 | 0 | −1 | 2 | −2 |
| I | −1 | −2 | −3 | −4 | −1 | −2 | −3 | −4 | −2 | 4 | 3 | −2 | 2 | 1 | −3 | −2 | −1 | −2 | −1 | 3 |
| L | −1 | −2 | −3 | −4 | −2 | −2 | −3 | −4 | −2 | 3 | 4 | −2 | 3 | 2 | −2 | −2 | −1 | −1 | 0 | 2 |
| K | 0 | 3 | 1 | 0 | −3 | 2 | 1 | −1 | 1 | −2 | −2 | 3 | −1 | −3 | −1 | 0 | 0 | −4 | −2 | −2 |
| M | −1 | −2 | −2 | −3 | −1 | −1 | −2 | −4 | −1 | 2 | 3 | −1 | 4 | 2 | −2 | −1 | −1 | −1 | 0 | 2 |
| F | −2 | −3 | −3 | −4 | −1 | −3 | −4 | −5 | 0 | 1 | 2 | −3 | 2 | 7 | −4 | −3 | −2 | 4 | 5 | 0 |
| P | 0 | −1 | −1 | −1 | −3 | 0 | 0 | −2 | −1 | −3 | −2 | −1 | −2 | −4 | 8 | 0 | 0 | −5 | −3 | −2 |
| S | 1 | 0 | 1 | 0 | 0 | 0 | 0 | 0 | 0 | −2 | −2 | 0 | −1 | −3 | 0 | 2 | 2 | −3 | −2 | −1 |
| T | 1 | 0 | 0 | 0 | 0 | 0 | 0 | −1 | 0 | −1 | −2 | 0 | −1 | −2 | 0 | 2 | 2 | −4 | −2 | 0 |
| W | −4 | −2 | −4 | −5 | −1 | −3 | −4 | −4 | −1 | −2 | −1 | −4 | −1 | 4 | −5 | −3 | −4 | 14 | 4 | −3 |
| Y | −2 | −2 | −1 | −3 | 0 | −2 | −3 | −4 | 2 | −1 | 0 | −2 | 0 | 5 | −3 | −2 | −2 | 4 | 8 | −1 |
| V | 0 | −2 | −2 | −3 | 0 | −2 | −2 | −3 | −2 | 3 | 2 | −2 | 2 | 0 | −2 | −1 | 0 | −3 | −1 | 3 |

For purposes herein, a “moderately conservative” replacement is any change having a nonnegative value in the PAM250 log-likelihood matrix reproduced herein above.

To avoid severely reducing or eliminating biological activity, amino acid residues that are conserved among the GP354 proteins of various species or among the Ig family members are not altered (except by conservative substitution) during genetic engineering. For instance, the cysteine residues for maintaining an Ig domain of GP354 should be conserved.

Relatedness of polynucleotides can also be characterized using a functional test, the ability of the two polynucleotides to base-pair to one another at defined hybridization stringencies. The invention thus provides isolated polynucleotides not only identical in sequence to those described with particularity herein, but also to provide isolated polynucleotides (“cross-hybridizing nucleic acids”) that hybridize under high stringency conditions (as defined herein) to all or to a portion of various of the isolated gp354 polynucleotides of the present invention (“reference nucleic acids”).

Such cross-hybridizing nucleic acids are useful, inter alia, as probes for, and to drive expression of, proteins related to the proteins of the present invention such as alternative splice variants and homologs (e.g., orthologs and paralogs). Particularly useful orthologs are those from other primate species, such as chimpanzee, rhesus macaque monkey, baboon, orangutan, and gorilla; from rodents, such as rats, mice, guinea pigs; from lagomorphs, such as rabbits, and from domestic livestock, such as cow, pig, sheep, horse, goat.

The hybridizing portion of the reference nucleic acid is typically at least 15 nucleotides in length, and often at least 17 , 20, 25, 30, 35, 40 or 50 nucleotides (nt) in length. Cross-hybridizing nucleic acids that hybridize to a larger portion of the reference nucleic acid—for example, to a portion of at least 50 nt, 100 nt, 150 nt, 200 nt, 250 nt, 300 nt, 350 nt, 400 nt, 450 nt, 500 nt or more, up to and including the entire length of the reference nucleic acid, are also useful.

The hybridizing portion of the cross-hybridizing nucleic acid is at least 75% identical in sequence to at least a portion of the reference nucleic acid. Typically, the hybridizing portion of the cross-hybridizing nucleic acid is at least 80%, often at least 85%, 86%, 87%, 88%, 89% or even at least 90% identical in sequence to at least a portion of the reference nucleic acid. Often, the hybridizing portion of the cross-hybridizing nucleic acid will be at least 91%, 92%, 93%, 94%, 95%, 96%, 97%, 98%, or 99% identical in sequence to at least a portion of the reference nucleic acid sequence. At times, the hybridizing portion of the cross-hybridizing nucleic acid will be at least 99.5% identical in sequence to at least a portion of the reference nucleic acid.

The invention also provides fragments of various of the isolated polynucleotides or nucleic acids of the present invention. By “fragments” of a reference nucleic acid is here intended isolated polynucleotides or nucleic acids, however obtained, that have a nucleotide sequence identical to a portion of the reference nucleic acid sequence, which portion is at least 17 nucleotides and less than the entirety of the reference nucleic acid.

In theory, an oligonucleotide of 17 nucleotides is of sufficient length as to occur at random less frequently than once in the three gigabases of the human genome, and thus to provide a nucleic acid probe that can uniquely identify the reference sequence in a nucleic acid mixture of mammalian genomic complexity. Further specificity can be obtained by probing nucleic acid samples of subgenomic complexity, and/or by using plural fragments as short as 17 nucleotides in length collectively to prime amplification of nucleic acids, as, e.g., by polymerase chain reaction (PCR).

The nucleic acid probes of the invention can be used to detect RNA transcripts or genomic sequences encoding homologs or identical proteins. The probe may comprise a label group attached thereto, e.g., a radioisotope, a fluorescent compound, an enzyme, or an enzyme co-factor. Such probes can be used as a part of diagnostic kit for identifying cells or tissues (i) that mis-express a GP354 protein (e.g., aberrant splicing, abnormal mRNA levels), or (ii) that harbor a mutation in the gp354 gene, such as a deletion, an insertion, or a point mutation. Such diagnostic kits preferably include labeled reagents and instructional inserts for their use.

The isolated polynucleotides of the invention can also be used as primers in PCR, primer extension and the like. To be useful as primers, the polynucleotides can be, e.g., at least 6 nucleotides (e.g., at least 7, 8, 9, or 10) in length. The primers can hybridize to an exonic sequence of a gp354 gene, for, e.g., amplification of a gp354 mRNA or cDNA. Alternatively, the primers can hybridize to an intronic sequence or an upstream or downstream regulatory sequence of a gp354 gene, to utilize non-transcribed, e.g., regulatory portions of the genomic structure of a gp354 gene.

The nucleic acid primers of the present invention can also be used, for example, to prime single base extension (SBE) for SNP detection (see, e.g., U.S. Pat. No. 6,004,744, the disclosure of which is incorporated herein by reference in its entirety). Isothermal amplification approaches, such as rolling circle amplification, are also now well-described. See, e.g., Schweitzer et al., Curr. Opin. Biotechnol. 12(1):21-7 (2001); U.S. Pat. Nos. 5,854,033 and 5,714,320 and international patent publications WO 97/19193 and WO 00/15779, the disclosures of which are incorporated herein by reference in their entireties. Rolling circle amplification can be combined with other techniques to facilitate SNP detection. See, e.g., Lizardi et al., Nature Genet. 19(3):225-32 (1998).

As described below, nucleic acid fragments that encode at least 6 contiguous amino acids (i.e., fragments of 18 nucleotides or more) are useful in directing the expression or the synthesis of peptides that have utility in mapping the epitopes of the protein encoded by the reference nucleic acid. See, e.g., Geysen et al., Proc. Natl. Acad. Sci. USA 81:3998-4002 (1984); and U.S. Pat. Nos. 4,708,871 and 5,595,915.

And, as described below, nucleic acid fragments that encode at least 8 contiguous amino acids (i.e., fragments of 24 nucleotides or more) are useful in directing the expression or the synthesis of peptides that have utility as immunogens. See, e.g., Lemer, “Tapping the immunological repertoire to produce antibodies of predetermined specificity,” Nature 299:592-596 (1982); Shinnick et al., Annu. Rev. Microbiol. 37:425-46 (1983); Sutcliffe et al., Science 219:660-6 (1983).

The nucleic acid fragment of the present invention is thus at least 17 nucleotides in length, typically at least 18 nucleotides in length, and often at least 24, 25, 30, 35, 40, or 45 nucleotides (nt) in length. Of course, larger fragments having at least 50 nt, 100 nt, 150 nt, 200 nt, 250 nt, 300 nt, 350 nt, 400 nt, 450 nt, 500 nt or more are also useful, and at times preferred, as will be appreciated by the skilled worker.

Having been based upon the mining of genomic sequence, rather than upon surveillance of expressed message, the present invention further provides isolated genome-derived polynucleotides or nucleic acids that include portions of the gp354 gene. The invention particularly provides genome-derived single exon probes, which comprise at least part of an exon (“reference exon”) and can hybridize detectably under high stringency conditions to transcript-derived nucleic acids that include the reference exon. The single exon probe will not, however, hybridize detectably under high stringency conditions to nucleic acids that lack the reference exon but include one or more exons that are found adjacent to the reference exon in the genome.

The present invention also provides isolated genome-derived polynucleotides or nucleic acids which include nucleic acid sequence elements that control transcription of the gp354 gene. Transcription control sequences include, e.g., promoters, enhancers, operators, terminators, silencers, and the like.

When desired for use in antisense inhibition of transcription or translation, or for antisense-mediated targeting of enzymatic nucleic acid molecules such as ribozymes, the isolated polynucleotides and nucleic acids of the present invention can usefully include one or more modified bases (see below) and/or one or more modified or altered internucleoside bonds, which often provide nuclease-resistance. See Hartmann et al. (eds.), Manual of Antisense Methodology (Perspectives in Antisense Science), Kluwer Law International (1999) (ISBN:079238539X); Stein et al. (eds.), Applied Antisense Oligonucleotide Technology, Wiley-Liss (cover (1998) (ISBN: 0471172790); Chadwick et al. (eds.), Oligonucleotides as Therapeutic Agents—Symposium No. 209, John Wiley & Son Ltd (1997) (ISBN: 0471972797). Such altered bases and internucleoside bonds are often desired also when the isolated nucleic acid of the present invention is to be used for targeted gene correction, as described in Gamper et al., Nucl. Acids Res. 28(21):4332-9 (2000), the disclosure of which is incorporated herein by reference in its entirety.

The antisense nucleic acid molecules (and enzymatic nucleic acids targeted by antisense) of the invention can be used in a therapeutic setting. These molecules can be expressed from an expression vector that contains an operably linked transcription regulatory sequence, the activity of which can be determined by the cell type into which the vector is introduced. For a discussion of the regulation of gene expression using antisense genes, see Weintraub et al., Antisense RNA as a molecular tool for genetic analysis, Trends. Genet. 1(1) (1986).

An antisense nucleic acid of the invention may be a ribozyme. Ribozymes are catalytic RNA molecules with ribonuclease activity that are capable of cleaving a single-stranded nucleic acid, such as an mRNA, to which they have a complementary region. Thus, ribozymes can be used to catalytically cleave gp354 mRNA transcripts to thereby inhibit translation of gp354 mRNA. A ribozyme having specificity for a gp354-encoding nucleic acid can be designed based upon the nucleotide sequence of a gp354 polynucleotide disclosed herein (i.e., SEQ ID NO:1 or 3).

Oligonucleotide mimetics of gp354, such as peptide nucleic acids (PNA), can be used in therapeutic and diagnostic applications. See, e.g., Hyrup et al. Bioorg. Med. Chem. Lett. 4:5-23 (1996). In PNA compounds, the phosphodiester backbone of the nucleic acid is replaced with an amide-containing backbone, in particular by repeating N-(2-aminoethyl) glycine units linked by amide bonds. PNAs For example, PNAs can be used as antisense or antigene agents for sequence-specific modulation of gene expression by, e.g., inducing transcription or translation arrest or inhibiting replication. PNAs of gp354 can also be used, e.g., in the analysis of single base pair mutations in a gene by, e.g., PNA directed PCR clamping; as artificial restriction enzymes when used in combination with other enzymes, e.g., S1 nucleases; or as probes or primers for DNA sequence and hybridization (Hyrup et al., supra; and Perry-O'Keefe, supra). PNAs of gp354 can be modified, e.g., to enhance their stability or cellular uptake, by attaching lipophilic or other helper groups to PNA, by the formation of PNA-DNA chimeras, or by the use of liposomes or other techniques of drug delivery known in the art (see infra).

Oligonucleotide of the invention may include other appended groups such as peptides (e.g., for targeting host cell receptors in vivo), or agents facilitating transport across the cell membrane or the blood-brain barrier. In addition, oligonucleotides can be modified with hybridization triggered cleavage agents or intercalating agents. To this end, the oligonucleotide may be conjugated to another molecule, e.g., a peptide, a hybridization triggered cross-linking agent, a transport agent, a hybridization-triggered cleavage agent, etc. (see infra).

Differences from nucleic acid compositions found in nature—e.g., non-native bases, altered internucleoside linkages, post-synthesis modification—can be present throughout the length of the gp354 polynucleotide or can usefully be localized to discrete portions thereof. As an example of the latter, chimeric nucleic acids can-be synthesized that have discrete DNA and RNA domains and demonstrated utility for targeted gene repair, as further described in U.S. Pat. Nos. 5,760,012 and 5,731,181, the disclosures of which are incorporated herein by reference in their entireties. Chimeric nucleic acids comprising both DNA and PNA have been demonstrated to have utility in modified PCR reactions. See Misra et al., Biochem. 37: 1917-1925 (1998); see also Finn et al, Nucl. Acids Res. 24: 3357-3363 (1996), incorporated herein by reference.

Polynucleotides and nucleic acids of the present invention can also usefully be bound to a substrate. The substrate can porous or solid, planar or non-planar, unitary or distributed; the bond can be covalent or noncovalent. Bound to a substrate, nucleic acids of the present invention can be used as probes in their unlabeled state. For example, the nucleic acids of the present invention can usefully be bound to a porous substrate, commonly a membrane, typically comprising nitrocellulose, nylon, or positively-charged derivatized nylon; so attached, the nucleic acids of the present invention can be used to detect gp354 nucleic acids present within a labeled nucleic acid sample, either a sample of genomic nucleic acids or a sample of transcript-derived nucleic acids, e.g. by reverse dot blot.

The nucleic acids of the present invention can also usefully be bound to a solid substrate, such as glass, although other solid materials, such as amorphous silicon, crystalline silicon, or plastics, can also be used. The nucleic acids of the present invention can be attached covalently to a surface of the support substrate or applied to a derivatized surface in a chaotropic agent that facilitates denaturation and adherence by presumed noncovalent interactions, or some combination thereof.

The nucleic acids of the present invention can be bound to a substrate to which a plurality of other nucleic acids are concurrently bound, hybridization to each of the plurality of bound nucleic acids being separately detectable. At low density, e.g. on a porous membrane, these substrate-bound collections are typically denominated macroarrays; at higher density, typically on a solid support, such as glass, these substrate bound collections of plural nucleic acids are colloquially termed microarrays. As used herein, the term microarray includes arrays of all densities. The invention thus provides microarrays that include the nucleic acids of the present invention.

The isolated nucleic acids of the present invention can be used as hybridization probes to detect, characterize, and quantify gp354 nucleic acids in, and isolate gp354 nucleic acids from, both genomic and transcript-derived nucleic acid samples. When free in solution, such probes are typically, but not invariably, detectably labeled; bound to a substrate, as in a microarray, such probes are typically, but not invariably unlabeled.

For example, the isolated nucleic acids of the present invention can be used as probes to detect and characterize gross alterations in the gp354 genomic locus, such as deletions, insertions, translocations, and duplications of the gp354 genomic locus through fluorescence in situ hybridization (FISH) to chromosome spreads. See, e.g., Andreeff et al. (eds.), Introduction to Fluorescence In Situ Hybridization: Principles and Clinical Applications, John Wiley & Sons (1999) (ISBN: 0471013455), the disclosure of which is incorporated herein by reference in its entirety. The isolated nucleic acids of the present invention can be used as probes to assess smaller genomic alterations using, e.g., Southern blot detection of restriction fragment length polymorphisms. The isolated nucleic acids of the present invention can be used as probes to isolate genomic clones that include the nucleic acids of the present invention, which thereafter can be restriction mapped and sequenced to identify deletions, insertions, translocations, and substitutions (single nucleotide polymorphisms, SNPs) at the sequence level.

The isolated nucleic acids of the present invention can be also be used as probes to detect, characterize, and quantify gp354 nucleic acids in, and isolate gp354 nucleic acids from, transcript-derived nucleic acid samples. For example, the isolated nucleic acids of the present invention can be used as hybridization probes to detect, characterize by length, and quantify gp354 mRNA by northern blot of total or poly-A⁺-selected RNA samples. The isolated nucleic acids of the present invention can also be used as hybridization probes to detect, characterize by location, and quantify gp354 message by in situ hybridization to tissue sections (see, e.g., Schwarchzacher et al., In Situ Hybridization, Springer-Verlag New York (2000) (ISBN: 0387915966), the disclosure of which is incorporated herein by reference in its entirety).

Further, the isolated nucleic acids of the present invention can be used as hybridization probes to measure the representation of gp354 clones in a cDNA library. For example, the isolated nucleic acids of the present invention can be used as hybridization probes to isolate gp354 nucleic acids from cDNA libraries, permitting sequence level characterization of gp354 RNA messages, including identification of deletions, insertions, truncations—including deletions, insertions, and truncations of exons in alternatively spliced forms—and single nucleotide polymorphisms.

As described in the Examples herein below, the nucleic acids of the present invention can also be used to detect and quantify gp354 nucleic acids in transcript-derived samples to measure expression of the gp354 gene. Measurement of gp354 expression has particular utility in diagnostic assays for conditions, disorders and diseases associated with abnormal gp354 expression, either in pancreatic and neural tissues where and in a manner in which it is normally expressed, as well as in tissues where it may be mis-expressed, as further described in the Examples herein below.

As would be readily apparent to one of skill in the art, each gp354 nucleic acid probe—whether labeled, substrate-bound, or both—is thus currently available for use as a tool for measuring the level of gp354 expression in pancreatic and neural tissues, in which expression has already been confirmed.

As for tissues not yet demonstrated to express gp354, the gp354 nucleic acid probes of the present invention are currently available as tools for surveying such tissues to detect the presence of gp354 nucleic acids, for example, to detect gp354 RNA expression in tissues of patients who present with a condition, disorder or disease associated with abnormal gp354 cellular expression in the pancreas or nervous system or abnormal tissue distribution in other tissues.

As noted above, the nucleic acid probes of the present invention are useful in constructing microarrays; the microarrays, in turn, are products of manufacture that are useful for measuring and for surveying gene expression in, for example, drug discovery and target validation programs. When included on a microarray, each gp354 nucleic acid probe makes the microarray specifically useful for detecting that portion of the gp354 gene included within the probe, thus imparting upon the microarray device the ability to detect a signal where, absent such probe, it would have reported no signal.

Changes in the level of gp354 expression need not be observed for the measurement of expression to have utility. Where gene expression analysis is used to assess toxicity of chemical agents on cells, for example, the failure of the agent to change a gene's expression level is evidence that the drug likely does not affect the pathway of which the gene's expressed protein is a part. Analogously, where gene expression analysis is used to assess side effects of pharmacologic agents—whether in lead compound discovery or in subsequent screening of lead compound derivatives—the inability of the agent to alter a gene's expression level is evidence that the drug does not affect the pathway of which the gene's expressed protein is a part. WO 99/58720, incorporated herein by reference in its entirety, provides methods for quantifying the relatedness of a first and second gene expression profile and for ordering the relatedness of a plurality of gene expression profiles, without regard to the identity or function of the genes whose expression is used in the calculation.

The genome-derived single exon probes and genome-derived single exon probe microarrays of the invention have the additional utility of permitting high-throughput detection of splice variants of the nucleic acids of the present invention.

Polynucleotides of the present invention, inserted into nucleic acid constructs such as vectors which flank the polynucleotide insert with a promoter can be used to drive in vitro expression of RNA complementary to either strand of the nucleic acid of the present invention. The RNA can be used as a single-stranded probe, in cDNA-mRNA subtraction, or for in vitro translation. Those polynucleotides which encode GP354 protein or portions thereof can further be used to express the GP354 proteins or protein fragments, either alone, or as part of fusion proteins. Expression can be from genomic or transcript-derived polynucleotides of the present invention.

Where protein expression is effected from genomic DNA, expression will typically be effected in eukaryotic, typically mammalian, cells capable of splicing introns from the initial RNA transcript. Expression can be driven from episomal vectors or from genomic DNA integrated into a host cell chromosome. As described below, where expression is from transcript-derived (or otherwise intron-less) polynucleotides of the invention, expression can be effected in a wide variety of prokaryotic or eukaryotic cells.

Expressed in vitro, the protein, protein fragment, or protein fusion can thereafter be isolated, to be used as a standard in immunoassays specific for the proteins, or protein isoforms, of the present invention; to be used as a therapeutic agent, e.g., to be administered as passive replacement therapy in individuals deficient in the proteins of the present invention; to be administered as a vaccine; to be used for in vitro production of specific antibody, the antibody thereafter to be used, e.g., as an analytical reagent for detection and quantitation of the proteins of the present invention or to be used as an immunotherapeutic agent.

The isolated polynucleotides and nucleic acids of the present invention can also be used to drive in vivo expression of the proteins of the present invention. In vivo expression can be driven from a vector—typically a viral vector, often a vector based upon a replication incompetent lentivirus, retrovirus, adenovirus, or adeno-associated virus (AAV)—for purpose of gene therapy. In vivo expression can be driven from expression control signals endogenous or exogenous (e.g., from a vector) to the nucleic acid. Other viral vectors of the invention include vectors derived, e.g., from baculoviruses, adenoviruses, parvoviruses, herpesviruses, poxviruses, adeno-associated viruses, Semliki Forest viruses, vaccinia viruses, and retroviruses.

Various forms of the isolated gp354 polynucleotides of the invention (e.g., genomic or cDNA) can be microinjected into male or female pronuclei, or can be integrated into embryonic stem (ES) cells to create transgenic non-human animals capable of producing the proteins of the present invention.

Genomic nucleic acids of the present invention can also be used to target homologous recombination to a gp354 locus in a subject. See, e.g., U.S. Pat. Nos. 6,187,305; 6,204,061; 5,631,153; 5,627,059; 5,487,992; 5,464,764; 5,614,396; 5,527,695 and 6,063,630; and Kmiec et al. (eds.), Gene Targeting Protocols, Vol. 133, Humana Press (2000) (ISBN: 0896033600); Joyner (ed.), Gene Targeting: A Practical Approach, Oxford University Press, Inc. (2000) (ISBN: 0199637938); Sedivy et al., Gene Targeting, Oxford University Press (1998) (ISBN: 071677013X); Tymms et al. (eds.), Gene Knockout Protocols, Humana Press (2000) (ISBN: 0896035727); Mak et al. (eds.), The Gene Knockout FactsBook, Vol. 2, Academic Press, Inc. (1998) (ISBN: 0124660444); Torres et al., Laboratory Protocols for Conditional Gene Targeting, Oxford University Press (1997) (ISBN: 019963677X); Vega (ed.), Gene Targeting, CRC Press, LLC (1994) (ISBN: 084938950X), the disclosures of which are incorporated herein by reference in their entireties.

Where the genomic region includes transcription regulatory elements, homologous recombination can be used to alter the expression of GP354, both for purpose of in vitro production of GP354 protein from human cells, and for purpose of gene therapy. See, e.g., U.S. Pat. Nos. 5,981,214, 6,048,524; 5,272,071; the disclosures of which are incorporated herein by reference in their entireties. Fragments of the polynucleotides of the present invention smaller than those typically used for homologous recombination can also be used for targeted gene correction or alteration, possibly by cellular mechanisms different from those engaged during homologous recombination. See, e.g., U.S. Pat. Nos. 5,945,339, 5,888,983, 5,871,984, 5,795,972, 5,780,296, 5,760,012, 5,756,325, 5,731,181; and Culver et al., “Correction of chromosomal point mutations in human cells with bifunctional oligonucleotides,” Nature Biotechnol. 17(10):989-93 (1999); Gamper et al., Nucl. Acids Res. 28(21):4332-9 (2000), the disclosures of which are incorporated herein by reference.

Polynucleotides of the present invention can be obtained by using the labeled probes of the present invention to probe nucleic acid samples, such as genomic libraries, cDNA libraries, and mRNA samples, by standard techniques. Polynucleotides of the present invention can also be obtained by amplification, using the nucleic acid primers of the present invention, as further demonstrated in Example 1, herein below. Polynucleotides of the present invention, especially if fewer than about 100 nucleotide, can also be synthesized chemically, typically by solid phase synthesis using commercially available automated synthesizers.

### Vectors and Host Cells

A. Nucleic Acid Constructs

The present invention provides nucleic acid constructs, such as vectors, that comprise one or more of the isolated polynucleotides of the invention, and host cells into which such vectors have been introduced.

The vectors can be used for propagating the polynucleotides of the present invention in host cells (cloning vectors), for shuttling the polynucleotides of the present invention between host cells derived from disparate organisms (shuttle vectors), for inserting the polynucleotides of the present invention into host cell chromosomes (insertion vectors), for expressing sense or antisense RNA transcripts of the polynucleotides of the present invention in vitro or within a host cell, and for expressing polypeptides encoded by the polynucleotides of the present invention, alone or as fusions to heterologous polypeptides (expression vectors). Vectors of the present invention will often be suitable for several such uses.

Vectors are by now well-known in the art, and are described, inter alia, in Jones et al. (eds.), Vectors: Cloning Applications: Essential Techniques (Essential Techniques Series), John Wiley & Son Ltd 1998 (ISBN: 047196266X); Jones et al. (eds.), Vectors: Expression Systems: Essential Techniques (Essential Techniques Series), John Wiley & Son Ltd, 1998 (ISBN:0471962678); Gacesa et al., Vectors: Essential Data, John Wiley & Sons, 1995 (ISBN: 0471948411); Cid-Arregui (eds.), Viral Vectors: Basic Science and Gene Therapy, Eaton Publishing Co., 2000 (ISBN: 188129935X); Sambrook et al., Molecular Cloning: A Laboratory Manual (3^{rd} ed.), Cold Spring Harbor Laboratory Press, 2001 (ISBN: 0879695773); Ausubel et al. (eds.), Short Protocols in Molecular Biology: A Compendium of Methods from Current Protocols in Molecular Biology (4^{th} ed.), John Wiley & Sons, 1999 (ISBN: 047132938X), the disclosures of which are incorporated herein by reference in their entireties. An enormous variety of vectors are available commercially. Use of existing vectors and modifications are well within the skill in the art.

Typically, vectors are derived from virus, plasmid, prokaryotic or eukaryotic chromosomal elements, or some combination thereof, and include at least one origin of replication, at least one site for insertion of heterologous nucleic acid, typically in the form of a polylinker with multiple, tightly clustered, single cutting restriction sites, and at least one selectable marker, although some integrative vectors will lack an origin that is functional in the host to be chromosomally modified, and some vectors will lack selectable markers. Vectors of the invention will further include at least one isolated polynucleotide nucleic acid of the invention inserted into the vector in at least one location. Where present, the origin of replication and selectable markers are chosen based upon the desired host cell or host cells; the host cells, in turn, are selected based upon the desired application.

For example, prokaryotic cells, typically E. coli, are typically chosen for cloning, i.e., for amplification of polynucleotide sequences in a host cell. In such case, vector replication is predicated on the replication strategies of coliform-infecting phage—such as phage lambda, M13, T7, T3 and P1—or on the replication origin of autonomously replicating episomes, notably the ColE 1 plasmid and later derivatives, including pBR322 and the pUC series plasmids. Where E. coli is used as host, selectable markers are, analogously, chosen for selectivity in gram negative bacteria: e.g., typical markers confer resistance to antibiotics, such as ampicillin, tetracycline, chloramphenicol, kanamycin, streptomycin, zeocin; auxotrophic markers can also be used.

As another example, yeast cells, typically S. cerevisiae, are chosen, inter alia, for eukaryotic genetic studies, for identification of interacting protein components, e.g. through use of a two-hybrid system, and for protein expression. Vectors of the present invention for use in yeast will typically, but not invariably, contain an origin of replication suitable for use in yeast and a selectable marker that is functional in yeast.

Examples of suitable yeast vectors include integrative YIp vectors, replicating episomal YEp vectors containing centromere sequences, CEN, and autonomously replicating sequences, ARS. YACs are based on yeast linear plasmids, denoted YLp, containing homologous or heterologous DNA sequences that function as telomeres (TEL) in vivo, as well as containing yeast ARS (origins of replication) and CEN (centromeres) segments.

Selectable markers in yeast vectors include a variety of auxotrophic markers, the most common of which are (in Saccharomyces cerevisiae) URA3, HIS3, LEU2, TRP1 and LYS2, which complement specific auxotrophic mutations, such as ura3-52, his3-D1, leu2-D1, trp1-D1 and lys2-201. The URA3 and LYS2 yeast genes further permit negative selection based on specific inhibitors, 5-fluoro-orotic acid (FOA) and α-aminoadipic acid (αAA), respectively, that prevent growth of the prototrophic strains but allows growth of the ura3 and lys2 mutants, respectively. Other selectable markers confer resistance to, e.g., zeocin.

Insect cells are often chosen for high efficiency protein expression. Where the host cells are from Spodoptera frugiperda—e.g., Sf9 and Sf21 cell lines, and expresSF™ cells (Protein Sciences Corp., Meriden, Conn., USA)—the vector replicative strategy is typically based upon the baculovirus life cycle. Typically, baculovirus transfer vectors are used to replace the wild-type AcMNPV polyhedrin gene with a heterologous gene of interest. Sequences that flank the polyhedrin gene in the wild-type genome are positioned 5′ and 3′ of the expression cassette on the transfer vectors. Following cotransfection with AcMNPV DNA, a homologous recombination event occurs between these sequences resulting in a recombinant virus carrying the gene of interest and the polyhedrin or p10 promoter. Selection can be based upon visual screening for lacZ fusion activity.

Mammalian cells are often chosen for expression of proteins intended as pharmaceutical agents, and are also chosen as host cells for screening of potential agonist and antagonists of a protein or a physiological pathway. Vectors intended for autonomous extrachromosomal replication in mammalian cells will typically include a viral origin, such as the SV40 origin (for replication in cell lines expressing the large T-antigen, such as COS1 and COS7 cells), the papillomavirus origin, or the EBV origin for long term episomal replication (for use, e.g., in 293-EBNA cells, which constitutively express the EBV EBNA-1 gene product and adenovirus E1A). Vectors intended for integration, and thus replication as part of the mammalian chromosome, can, but need not, include an origin of replication functional in mammalian cells, such as the SV40 origin. Vectors based upon viruses, such as lentiviruses, adenovirus, adeno-associated virus, vaccinia virus, and various mammalian retroviruses, will typically replicate according to the viral replicative strategy.

Selectable markers for use in mammalian cells include resistance to neomycin (G418), blasticidin, hygromycin and to zeocin, and selection based upon the purine salvage pathway using HAT medium.

Plant cells can also be used for expression, with the vector replicon typically derived from a plant virus (e.g., cauliflower mosaic virus, CaMV; tobacco mosaic virus, TMV) and selectable markers chosen for suitability in plants.

For propagation of polynucleotides of the present invention that are larger than can readily be accommodated in vectors derived from plasmids or virus, the invention further provides artificial chromosomes—BACs, YACs, and HACs—that comprise gp354 nucleic acids, often genomic nucleic acids.

For propagation of polynucleotides of the present invention that are larger than can readily be accommodated in vectors derived from plasmids or viruses, the invention further provides artificial chromosomes—BACs, YACs, and HACs—that comprise gp354 nucleic acids, often genomic nucleic acids. See, e.g., Shizuya et al., Keio J Med. 50(1):26-30 (2001); Shizuya et al., Proc. Natl. Acad. Sci. USA 89(18):8794-7 (1992); Kuroiwa et al., Nature Biotechnol. 18(10):1086-90 (2000); Henning et al., Proc. Natl. Acad. Sci. USA 96(2):592-7 (1999); Harrington et al., Nature Genet. 15(4):345-55 (1997), the disclosures of which are incorporated herein by reference.

Vectors of the invention will also often include elements that permit in vitro transcription of RNA from the inserted heterologous nucleic acid. Such vectors typically include a phage promoter, such as that from T7, T3, or SP6, flanking the nucleic acid insert. Often two different such promoters flank the inserted nucleic acid, permitting separate in vitro production of both sense and antisense strands.

Expression vectors of the invention which will drive expression of polypeptides from the inserted heterologous nucleic acid will often include a variety of other genetic elements operatively linked to the protein-encoding heterologous nucleic acid insert, typically genetic elements that drive and regulate transcription, such as promoters and enhancer elements, those that facilitate RNA processing, such as transcription termination, splicing signals and/or polyadenylation signals, and those that facilitate translation, such as ribosomal consensus sequences. Other transcription control sequences include, e.g., operators, silencers, and the like. Use of such expression control elements, including those that confer inducible expression, and developmental or tissue-regulated expression are well-known in the art.

Tissue-specific regulatory elements capable of expressing GP354 in the pancreas, nervous system or mammary glands may be particularly useful and are known in the art, e.g., the neuron-specific neurofilament promoter (Byrne and Ruddle Proc. Natl. Acad. Sci. USA 86:5473-5477 (1989)), a pancreas-specific promoter (Edlund et al. Science 230:912-916 (1985)), and mammary gland-specific promoters (e.g., milk whey promoter; U.S. Pat. No. 4,873,316 and European Application Publication No. 264,166). Developmentally-regulated promoters may also be selected, including but not limited to the murine hox promoters (Kessel and Gruss Science 249:374-379 (1990)) and the α-fetoprotein promoter (Campes and Tilghman Genes Dev. 3:537-546 (1989)). A huge variety of inducible promoters are known and may be selected based on the particular application.

Expression vectors can be designed to fuse the expressed polypeptide to small protein tags that facilitate purification and/or visualization. Many such tags are known and available. Expression vectors can also be designed to fuse proteins encoded by the heterologous nucleic acid insert to polypeptides larger than purification and/or identification tags. Useful protein fusions include those that permit display of the encoded protein on the surface of a phage or cell, fusions to intrinsically fluorescent proteins, such as luciferase or those that have a green fluorescent protein (GFP)-like chromophore, fusions to the IgG Fc region or other immunoglobulin type constant domains, and fusions for use in two hybrid selection systems.

For secretion of expressed proteins, a wide variety of vectors are available which include appropriate sequences that encode secretion signals, such as leader peptides. Vectors designed for phage display, yeast display, and mammalian display, for example, target recombinant proteins using an N-terminal cell surface targeting signal and a C-terminal transmembrane anchoring domain.

A wide variety of vectors now exist that fuse proteins encoded by heterologous nucleic acids to the chromophore of the substrate-independent, intrinsically fluorescent green fluorescent protein from Aequorea Victoria (“GFP”) and its many color-shifted and/or stabilized variants.

Vectors which allow fusions of heterologous sequences to the IgG Fc region to increase serum half-life of protein pharmaceutical products through interaction with the FcRn receptor (also denominated the FcRp receptor and the Brambell receptor, FcRb), are also widely available.

For long-term, high-yield recombinant production of the proteins, protein fusions, and protein fragments of the present invention, stable expression is preferred. Stable expression is readily achieved by integration into the host cell genome of vectors (preferably having selectable markers), followed by selection for integrants.

B. Host Cells

The present invention further includes host cells—either prokaryotic (bacteria) or eukaryotic (e.g., yeast, insect, plant and animal cells)—comprising the nucleic acid constructs such as vectors of the present invention, either present episomally within the cell or integrated, in whole or in part, into the host cell chromosome.

Among other considerations, some of which are described above, a host cell strain may be chosen for its ability to process the expressed protein in the desired fashion. Such post-translational modifications of the polypeptide include, but are not limited to, acetylation, carboxylation, glycosylation, phosphorylation, lipidation, and acylation, and it is an aspect of the present invention to provide GP354 proteins with such post-translational modifications.

Representative, non-limiting examples of appropriate host cells include bacterial cells, such as E. coli, Caulobacter crescentus, Streptomyces species, and Salmonella typhimurium; yeast cells, such as Saccharomyces cerevisiae, Schizosaccharomyces pombe, Pichia pastoris, Pichia methanolica; insect cell lines, such as those from Spodoptera frugiperda—e.g., Sf9 and Sf21 cell lines, and expresSF™ cells (Protein Sciences Corp., Meriden, Conn., USA)—Drosophila S2 cells, and Trichoplusia ni High Five® Cells (Invitrogen, Carlsbad, Calif., USA); and mammalian cells. Typical mammalian cells include COS1 and COS7 cells, chinese hamster ovary (CHO) cells, NIH 3T3 cells, 293 cells, HEPG2 cells, HeLa cells, L cells, HeLa, MDCK, HEK293, W138, murine ES cell lines (e.g., from strains 129/SV, C57/BL6, DBA-1, 129/SVJ), K562, Jurkat cells, and BW5147. Other useful mammalian cell lines are well known and readily available from the American Type Culture Collection (ATCC) (Manassas, Va., USA) and the National Institute of General medical Sciences (NIGMS) Human Genetic Cell Repository at the Coriell Cell Repositories (Camden, N.J., USA).

Methods for introducing the vectors and nucleic acids of the present invention into the host cells are well known in the art; the choice of technique will depend primarily upon the specific vector to be introduced and the host cell chosen.

### GP354 Proteins Polypeptides and Fragments

The present invention provides GP354 proteins and various fragments thereof suitable for use as antigens (e.g., for epitope mapping), for use as immunogens (e.g., for raising antibodies or as vaccines), and for use in therapeutic compositions. Also provided are fusions of GP354 polypeptides and fragments to heterologous polypeptides, and conjugates of the proteins, fragments, and fusions of the present invention to other moieties (e.g., to carrier proteins, to fluorophores).

In some embodiments, the invention provides an isolated GP354 polypeptide comprising the amino acid sequence encoded by a full-length gp354 cDNA (SEQ ID NO:1, 7 or 11), or a degenerate variant. The invention also provides an isolated GP354 polypeptide having the amino acid sequence encoded by a full-length gp354 cDNA (SEQ ID NO:1, 7 or 11), optionally having one or more conservative amino acid substitutions.

The invention also provides an isolated GP354 polypeptide comprising the amino acid sequence encoded by a polynucleotide sequence that hybridizes under high stringency conditions to a probe having part or all of the nucleotide sequence of a gp354 cDNA (SEQ ID NO:1, 7 or 11). Preferably, an isolated GP354 polypeptide encoded by a stringently or moderately stringent cross-hybridizing polynucleotide of the invention will have at least one biological activity of GP354.

In another series of embodiments, the invention provides an isolated GP354 polypeptide comprising the GP354 amino acid sequence of SEQ ID NO:2, 8 or 12, optionally having one or more conservative amino acid substitutions. Also provided is an isolated GP354 polypeptide having the amino acid sequence encoded by the GP354 polypeptide sequence of SEQ ID NO:2, 8 or 12, optionally having one or more conservative amino acid substitutions. The invention further provides fragments of each of the above-described isolated polypeptides, particularly fragments having at least 6 amino acids, 8 amino acids, 15 amino acids up to the entirety of the sequence given in SEQ ID NO:2, 8 or 12.

Each of the above isolated polypeptides includes an N-terminal 18 or 21 amino acid signal sequence which is typically removed upon insertion of the protein through a membrane. Accordingly, the invention provides the above isolated GP354 polypeptides from which the N-terminal signal sequence has been removed. Cleavage is predicted to occur between the G and P residues at positions 18-19 of SEQ ID NO:2 or at positions 21-22 of SEQ ID NO:8.

The invention thus provides an isolated GP354 polypeptide comprising all or a portion of the predicted mature N-terminal extracellular domain of GP354. (See FIGS. 1 and 5; SEQ ID NOs:2 and 8 for GP354 domains and sequences). The predicted mature extracellular domain of GP354 (i.e., lacking the secretion signal sequence), consists of amino acids 19-507 of SEQ ID NO:2, or of amino acids 22-510 of SEQ ID NO:8. Also included are fragments of the above sequences having at least 6 amino acids, 8 amino acids, 15 amino acids up to the entirety of the specified sequence.

The invention also provides an isolated GP354 polypeptide comprising or having all or a portion of the N-terminal extracellular domain of GP354. (See FIGS. 1 and 5; SEQ ID NOs:2 and 8 for GP354 domains and sequences). The N-terminal extracellular domain of GP354 consists of amino acids 1-507 of SEQ ID NO:2, or of amino acids 1-510 of SEQ ID NO:8. Also included are fragments of the above sequences having at least 6 amino acids, 8 amino acids, 15 amino acids up to the entirety of the specified sequence.

In preferred embodiments, the isolated GP354 polypeptide has or comprises the entire extracellular domain of GP354 and lacks a functional GP354 transmembrane domain. The transmembrane domain may either be excluded, deleted or mutated to render it non-functional. The transmembrane domain of GP354 consists of amino acids 508-530 of SEQ ID NO:2, or of amino acids 511-533 of SEQ ID NO:8.

In other preferred embodiments, the isolated GP354 polypeptide consists of part or all of the GP354 N-terminal extracellular domain fused to a heterologous protein domain. Preferably, the isolated GP354 polypeptide comprises at least one extracellular Ig domain, more preferably comprises two GP354 extracellular Ig domains, and most preferably comprises three, four or five GP354 extracellular Ig domains.

Also preferred is an isolated GP354 polypeptide comprising a GP354 fragment selected from the group consisting of the transmembrane domain of GP354 and the C-terminal cytoplasmic region of GP354. In other preferred embodiments, the isolated GP354 polypeptide consists of part or all of the GP354 cytoplasmic or transmembrane domains fused to a heterologous protein domain.

The GP354 fragments of the invention may be continuous portions of the native GP354 protein. However, it will be appreciated that knowledge of the GP354 gene and protein sequences as provided herein permits recombining of various domains that are not contiguous in the native GP354 protein.

The invention also provides polypeptides comprising select portions of GP354 and related proteins. As will be further discussed herein below, these protein fragments, especially when coupled to heterologous protein fragments, can be used, for example, to target agents to particular cell types through protein-protein interaction; to inhibit protein-protein interactions between Ig domain containing proteins; for competitive binding assays; and to raise fragment-specific GP354 antibodies.

In a first series of such embodiments, the protein fragment comprises, in at least one copy, one, two, three, four or five of the Ig domains characteristic of the N-terminal extracellular portion of GP354. Specifically, the five extracellular Ig domains are encoded by amino acids 35-102, 136-203, 239-290, 323-374 and 410-485, respectively, of the GP354 amino acid sequence of SEQ ID NO:2 (see FIG. 1), and are encoded by amino acids 38-109, 139-206, 242-293, 326-377 and 413-488, respectively, of the GP354 amino acid sequence of SEQ ID NO:8 (see FIG. 5). In preferred embodiments, the protein fragment encodes at least two, preferably three, more preferably four and most preferably all five domains in at least one copy.

Preferably, the protein fragment contains an N-terminal signal secretion sequence that will mediate transport of the polypeptide through a membrane. The GP354 signal secretion sequence is encoded by amino acids 1-18 of the GP354 amino acid sequence of SEQ ID NO:2 (see FIG. 1) and by amino acids 1-21 of SEQ ID NO:8 (see FIG. 5). More preferably, the signal secretion sequence of the protein fragment is from GP354.

The above preferred protein fragments may optionally include a transmembrane domain, if insertion of the polypeptide into a membrane is so-desired. The transmembrane domain may be a GP354 domain (see below) or may be encoded by a heterologous gene encoding a transmembrane domain of a heterologous membrane-associated protein.

If so-desired, the above preferred protein fragments may further comprise an intracellular C-terminal domain if specific signaling reactions are desired in response to GP354 binding interactions. The intracellular domain may be derived from GP354 (see below) or may be encoded by a heterologous gene encoding an intracellular domain of a heterologous membrane-associated protein.

Other preferred embodiments of the protein fragments of the invention are those that comprise the transmembrane domain of GP354. Specifically, the GP354 transmembrane domain is encoded by amino acids 508-530 of the GP354 amino acid sequence of SEQ ID NO:2 (see FIG. 1).

Yet other preferred embodiments of the above-described protein fragments have a C-terminal intracellular domain of GP354. Specifically, one intracellular domain of GP354 is encoded by amino acids 531-592 of the GP354 amino acid sequence of SEQ ID NO:2 (see FIG. 1). Another form of an intracellular domain of GP354 is encoded by amino acids 534-708 of the GP354 amino acid sequence of SEQ ID NO:8 (see FIG. 5). It is believed that these different intracellular domain forms may be produced by alternative splicing.

A preferred protein fragment of the invention is encoded by nucleotides 139-923 of the gp354 cDNA sequence of SEQ ID NO:1 (see FIG. 1). It is encoded by an RT-PCR fragment amplified from pancreatic RNA using primers GX1-218 (SEQ ID NO:16) and GX1-219 (SEQ ID NO:17; see Example 2) and consists of amino acids 47-307 of SEQ ID NO:2, i.e., it encodes most of the first N-terminal Ig domain (missing the first 12 of 68 amino acids), and the second and third Ig domains of GP354.

As described above, the invention further provides proteins that differ in sequence from those described with particularity in the above-referenced SEQ ID NOs, whether by way of insertion or deletion, by way of conservative or moderately conservative substitutions, as hybridization related proteins, or as cross-hybridizing proteins, with those that substantially retain a GP354 activity preferred. As also discussed above, the invention further provides fusions of the polypeptides, proteins and protein fragments herein described to heterologous polypeptides.

When used as immunogens, the various protein embodiments of the present invention can be used, inter alia, to elicit antibodies that bind to a variety of epitopes of the GP354 protein.

### Other Defining Characteristics of GP354 Proteins

FIG. 1 presents the deduced amino acid sequences (SEQ ID NO:2) encoded by the gp354 cDNA clone (SEQ ID NO:1). Similarly, the amino acid sequences presented in SEQ ID NOs:4, 8, 10 and 12 are deduced from the nucleotide sequences presented in SEQ ID NOs:3, 7, 9 and 11, respectively. Unless otherwise indicated, amino acid sequences of the proteins of the present invention were determined as a predicted translation from a nucleic acid sequence. Accordingly, any amino acid sequence presented herein may contain errors due to errors in the nucleic acid sequence, as described in detail above. Furthermore, single nucleotide polymorphisms (SNPs) occur frequently in eukaryotic genomes—more than 1.4 million SNPs have already identified in the human genome, International Human Genome Sequencing Consortium, Nature 409:860-921 (2001)—and the sequence determined from one individual of a species may differ from other allelic forms present within the population. Small deletions and insertions can often be found that do not alter the function of the protein.

Accordingly, the present invention provides GP354 polypeptides not only identical in sequence to those described with particularity herein, but also isolated proteins at least about 80% identical in sequence to those described with particularity herein, typically at least about 85%, 90%, 91%, 92%, 93%, 94%, or 95% identical in sequence to those described with particularity herein, usefully at least about 96%, 97%, 98%, or 99% identical in sequence to those described with particularity herein, and, most conservatively, at least about 99.5%, 99.6%, 99.7%, 99.8% and 99.9% identical in sequence to those described with particularity herein. These sequence variants can be naturally occurring or can result from human intervention by way of random or directed mutagenesis.

For purposes herein, percent identity of two amino acid sequences is determined using the procedure of Tatiana et al., “Blast 2 sequences—a new tool for comparing protein and nucleotide sequences”, FEMS Microbiol Lett. 174:247-250 (1999), which procedure is effectuated by the computer program Blast 2 SEQUENCES, available online at the National Center for Biotechnology Information.

To assess percent identity of amino acid sequences, the BlastP module of Blast 2 SEQUENCES is used with default values of (i) BLOSUM62 matrix, Henikoff et al., Proc. Natl. Acad. Sci USA 89(22):10915-9 (1992); (ii) open gap 11 and extension gap 1 penalties; and (iii) gap x_dropoff 50 expect 10 word size 3 filter, and both sequences are entered in their entireties.

As is well known, amino acid substitutions occur frequently among natural allelic variants, with conservative substitutions often occasioning only de minimis change in protein function. Accordingly, the present invention provides proteins not only identical in sequence to those described with particularity herein, but also isolated proteins having the sequence of GP354 proteins, or portions thereof, with conservative amino acid substitutions. Also provided are isolated proteins having the sequence of GP354 proteins, and portions thereof, with moderately conservative amino acid substitutions. These conservatively-substituted or moderately conservatively-substituted variants can be naturally occurring or can result from human intervention.

Allelic variation may account for differences in amino acid sequence between SEQ ID NO:2 and SEQ ID NO:8 at positions 195, 196, 539 and 540, for example. Splice variants (e.g., differential 5′ or 3′ splice site selection) may also account for the differences between the C-terminal amino acid sequences of SEQ ID NO:2 and SEQ ID NO:8.

As is also well known in the art, relatedness of proteins can also be characterized using a functional test, the ability of the encoding nucleic acids to base-pair to one another at defined hybridization stringencies. It is, therefore, another aspect of the invention to provide isolated proteins not only identical in sequence to those described with particularity herein, but also to provide isolated proteins (“hybridization related proteins”) that are encoded by nucleic acids that hybridize under high stringency conditions (as defined herein above) to all or to a portion of various of the isolated polynucleotides of the present invention (“reference nucleic acids”).

The hybridization related proteins can be alternative isoforms, homologs, paralogs, and orthologs of the GP354 protein of the present invention. Particularly useful orthologs are those from other primate species, such as chimpanzee, rhesus macaque monkey, baboon, orangutan, and gorilla; from rodents, such as rats, mice, guinea pigs; from lagomorphs, such as rabbits, and from domestic livestock, such as cow, pig, sheep, horse, goat.

Relatedness of proteins can also be characterized using a second functional test, the ability of a first protein to inhibit competitively the binding of a second protein to an antibody. It is, therefore, another aspect of the present invention to provide isolated proteins not only identical in sequence to those described with particularity herein, but also to provide isolated proteins (“cross-reactive proteins”) that competitively inhibit the binding of antibodies to all or to a portion of various of the isolated GP354 proteins of the present invention (“reference proteins”). Such competitive inhibition can readily be determined using immunoassays well known in the art.

Among the proteins of the present invention that differ in amino acid sequence from those described with particularity herein—including those that have deletions and insertions causing up to 10% non-identity, those having conservative or moderately conservative substitutions, hybridization related proteins, and cross-reactive proteins—those that substantially retain one or more GP354 activities are preferred (see supra).

Residues that are tolerant of change while retaining function can be identified by altering the protein at known residues using methods known in the art, such as alanine scanning mutagenesis, Cunningham et al., Science 244(4908): 1081-5 (1989); transposon linker scanning mutagenesis, Chen et al., Gene 263(1-2):39-48 (2001); combinations of homolog- and alanine-scanning mutagenesis, Jin et al., J. Mol. Biol. 226(3):851-65 (1992); combinatorial alanine scanning, Weiss et al., Proc. Natl. Acad. Sci. USA 97(16):8950-4 (2000), followed by functional assay. Transposon linker scanning kits are available commercially (New England Biolabs, Beverly, Mass., USA, catalog. no. E7-102S; EZ::TN™ In-Frame Linker Insertion Kit, catalog no. EZI04KN, Epicentre Technologies Corporation, Madison, Wis., USA).

As further described below, the isolated proteins of the present invention can readily be used as specific immunogens to raise antibodies that specifically recognize GP354 proteins, their isoforms, homologs, paralogs, and/or orthologs. The antibodies, in turn, can be used, inter alia, specifically to assay for the GP354 proteins of the present invention—e.g. by ELISA for detection of protein fluid samples, such as serum, by immunohistochemistry or laser scanning cytometry, for detection of protein in tissue samples, or by flow cytometry, for detection of intracellular protein in cell suspensions—for specific antibody-mediated isolation and/or purification of GP354 proteins, as for example by immunoprecipitation, and for use as specific agonists or antagonists of GP354 action.

The isolated proteins of the present invention are also immediately available for use as specific standards in assays used to determine the concentration and/or amount specifically of the GP354 proteins of the present invention. As is well known, ELISA kits for detection and quantitation of protein analytes typically include isolated and purified protein of known concentration for use as a measurement standard (e.g., the human interferon-γ OptElA kit, catalog no. 555142, Pharmingen, San Diego, Calif., USA includes human recombinant gamma interferon, baculovirus produced).

The isolated proteins of the present invention are also immediately available for use as specific biomolecule capture probes for surface-enhanced laser desorption ionization (SELDI) detection of protein-protein interactions, WO 98/59362; WO 98/59360; WO 98/59361; and Merchant et al., Electrophoresis 21(6): 1164-77 (2000), the disclosures of which are incorporated herein by reference in their entireties. Analogously, the isolated proteins of the present invention are also immediately available for use as specific biomolecule capture probes on BIACORE surface plasmon resonance probes. See Weinberger et al., Pharmacogenomics 1(4):395-416 (2000); Malmqvist, Biochem. Soc. Trans. 27(2):335-40 (1999).

The isolated proteins of the present invention are also useful as a therapeutic supplement in patients diagnosed to have a specific deficiency in GP354 production or activity.

The invention also provides fragments of various of the proteins of the present invention. The protein fragments are useful as antigenic and immunogenic fragments of GP354. By “fragments” of a protein is here intended isolated proteins (equally, polypeptides, peptides, oligopeptides), however obtained, that have an amino acid sequence identical to a portion of the reference amino acid sequence, which portion is at least 6 amino acids and less than the entirety of the reference nucleic acid. As so defined, “fragments” need not be obtained by physical fragmentation of the reference protein, although such provenance is not thereby precluded.

Fragments of at least 6 contiguous amino acids are useful in mapping B cell and T cell epitopes of the reference protein. See, e.g., Geysen et al., “Use of peptide synthesis to probe viral antigens for epitopes to a resolution of a single amino acid,” Proc. Natl. Acad. Sci. USA 81:3998-4002 (1984) and U.S. Pat. Nos. 4,708,871 and 5,595,915, the disclosures of which are incorporated herein by reference in their entireties. Because the fragment need not itself be immunogenic, part of an immunodominant epitope, nor even recognized by native antibody, to be useful in such epitope mapping, all fragments of at least 6 amino acids of the proteins of the present invention have utility in such a study.

Fragments of at least eight contiguous amino acids, often at least fifteen contiguous amino acids, have utility as immunogens for raising antibodies that recognize the proteins of the present invention. See, e.g., Lemer, “Tapping the immunological repertoire to produce antibodies of predetermined specificity,” Nature 299:592-596 (1982); Shinnick et al., “Synthetic peptide immunogens as vaccines,” Annu. Rev. Microbiol. 37:425-46 (1983); Sutcliffe et al., “Antibodies that react with predetermined sites on proteins,” Science 219:660-6 (1983), the disclosures of which are incorporated herein by reference in their entireties. As further described in the above-cited references, virtually all 8-mers, conjugated to a carrier, such as a protein, prove immunogenic—that is, prove capable of eliciting antibody for the conjugated peptide; accordingly, all fragments of at least 8 amino acids of the proteins of the present invention have utility as immunogens.

Fragments of at least 8, 9, 10 or 12 contiguous amino acids are also useful as competitive inhibitors of binding of the entire protein, or a portion thereof, to antibodies (as in epitope mapping), and to natural binding partners, such as subunits in a multimeric complex or to receptors or ligands of the subject protein; this competitive inhibition permits identification and separation of molecules that bind specifically to the protein of interest, U.S. Pat. Nos. 5,539,084 and 5,783,674, incorporated herein by reference in their entireties.

The protein, or protein fragment, of the present invention is thus at least 6 amino acids in length, typically at least 8, 9, 10 or 12 amino acids in length, and often at least 15 amino acids in length. Often, the protein or the present invention, or fragment thereof, is at least 20, 25, 30, 35, or 50 amino acids or more in length. Larger fragments having at least 75, 100, 150 or more amino acids are also useful, and at times preferred.

The present invention further provides fusions of each of the GP354 proteins and protein fragments of the present invention to heterologous polypeptides. By fusion is here intended that the protein or protein fragment of the present invention is linearly contiguous to the heterologous polypeptide in a peptide-bonded polymer of amino acids or amino acid analogues; by “heterologous polypeptide” is here intended a polypeptide that does not naturally occur in contiguity with the protein or protein fragment of the present invention. As so defined, the fusion can consist entirely of a plurality of fragments of the GP354 protein in altered arrangement; in such case, any of the GP354 fragments can be considered heterologous to the other GP354 fragments in the fusion protein. More typically, however, the heterologous polypeptide is not drawn from the GP354 protein itself.

The fusion proteins of the present invention will include at least one fragment of the protein of the present invention, which fragment is at least 6, typically at least 8, often at least 15, and usefully at least 16, 17, 18, 19, or 20 amino acids long. The fragment of the protein of the present to be included in the fusion can usefully be at least 25, 50, 75, 100, or 150 amino acids long. Fusions that include the entirety of the GP354 proteins of the invention, or functional domains, such as the N-terminal GP354 Ig domains and the C-terminal intracellular domain have particular utility. Fusions comprising GP354 Ig domains will be useful in engineering fusion proteins that will recognize other Ig domain-containing molecules and cells that displaying them on their surface. This, in turn, may be useful for targeting a heterologous sequence, such as a toxin or a therapeutic, to a pancreatic cell or a CNS-derived cell that expressed GP354 or a binding partner; or to all or a portion of a cell surface molecule derived from a pancreatic cell or a CNS-derived cell that expresses GP354 or a binding partner.

The heterologous polypeptide included within the fusion protein of the present invention is at least 6 amino acids in length, often at least 8 amino acids in length, and preferably, at least 15, 20, and 25 amino acids in length. Fusions that include larger polypeptides, such as the IgG Fc region, and even entire proteins (such as luciferase or GFP chromophore-containing proteins), have particular utility.

As described above in the description of vectors and expression vectors of the present invention, heterologous polypeptides included in the fusion proteins of the present invention usefully include those designed to facilitate purification and/or visualization of recombinantly-expressed proteins. Although purification tags can also be incorporated into fusions that are chemically synthesized, chemical synthesis typically provides sufficient purity that further purification by HPLC suffices; however, visualization tags as above described retain their utility even when the protein is produced by chemical synthesis, and when so included render the fusion proteins of the present invention useful as directly detectable markers of GP354 presence.

As also discussed above, heterologous polypeptides to be included in the fusion proteins of the present invention can usefully include those that facilitate secretion of recombinantly expressed proteins—into the periplasmic space or extracellular milieu for prokaryotic hosts, into the culture medium for eukaryotic cells—through incorporation of secretion signals and/or leader sequences.

Other useful protein fusions of the present invention include those that permit use of the protein of the present invention as bait in a yeast two-hybrid system. See Bartel et al. (eds.), The Yeast Two-Hybrid System, Oxford University Press (1997) (ISBN: 0195109384); Zhu et al., Yeast Hybrid Technologies, Eaton Publishing, (2000) (ISBN 1-881299-15-5); Fields et al., Trends Genet. 10(8):286-92 (1994); Mendelsohn et al., Curr. Opin. Biotechnol. 5(5):482-6 (1994); Luban et al., Curr. Opin. Biotechnol. 6(1):59-64 (1995); Allen et al., Trends Biochem. Sci. 20(12):511-6 (1995); Drees, Curr. Opin. Chem. Biol. 3(1):64-70 (1999); Topcu et al., Pharm. Res. 17(9):1049-55 (2000); Fashena et al., Gene 250(1-2):1-14 (2000), the disclosures of which are incorporated herein by reference in their entireties. Typically, such fusion is to either E. coli LexA or yeast GAL4 DNA binding domains. Related bait plasmids are available that express the bait fused to a nuclear localization signal.

Other useful protein fusions include those that permit display of the encoded protein on the surface of a phage or cell, fusions to intrinsically delectable proteins, such as fluorescent or light-emitting proteins, and fusions to stable protein domains such as an immunoglobulin heavy chain domain like the IgG Fc region, as described above.

The proteins and protein fragments of the present invention can also usefully be fused to protein toxins, such as Pseudomonas exotoxin A, diphtheria toxin, shiga toxin A, anthrax toxin lethal factor, ricin, or other biologically deleterious moieties in order to effect specific ablation of cells that bind or take up the proteins of the present invention.

The isolated proteins, protein fragments, and protein fusions of the present invention can be composed of natural amino acids linked by native peptide bonds, or can contain any or all of nonnatural amino acid analogues, nonnative bonds, and post-synthetic (post-translational) modifications, either throughout the length of the protein or localized to one or more portions thereof.

As is well known in the art, when the isolated protein is used, e.g., for epitope mapping, the range of such nonnatural analogues, nonnative inter-residue bonds, or post-synthesis modifications will be limited to those that permit binding of the peptide to antibodies. When used as an immunogen for the preparation of antibodies in a non-human host, such as a mouse, the range of such nonnatural analogues, nonnative inter-residue bonds, or post-synthesis modifications will be limited to those that do not interfere with the immunogenicity of the protein. When the isolated protein is used as a therapeutic agent, such as a vaccine or for replacement therapy, the range of such changes will be limited to those that do not confer toxicity upon the isolated protein.

Techniques for incorporating non-natural amino acids during solid phase chemical synthesis or by recombinant methods are well established in the art. Procedures are described, inter alia, in Chan et al. (eds.), Fmoc Solid Phase Peptide Synthesis: A Practical Approach (Practical Approach Series), Oxford Univ. Press (March 2000) (ISBN: 0199637245); Jones, Amino Acid and Peptide Synthesis (Oxford Chemistry Primers, No 7), Oxford Univ. Press (August 1992) (ISBN: 0198556683); and Bodanszky, Principles of Peptide Synthesis (Springer Laboratory), Springer Verlag (December 1993) (ISBN: 0387564314), the disclosures of which are incorporated herein by reference in their entireties.

D-enantiomers of natural amino acids can readily be incorporated during chemical peptide synthesis: peptides assembled from D-amino acids are more resistant to proteolytic attack; incorporation of D-enantiomers can also be used to confer specific three dimensional conformations on the peptide. Other amino acid analogues commonly added during chemical synthesis include ornithine, norleucine, phosphorylated amino acids (typically phosphoserine, phosphothreonine, phosphotyrosine), L-malonyltyrosine, a non-hydrolyzable analog of phosphotyrosine (Kole et al., Biochem. Biophys. Res. Com. 209:817-821 (1995)), and various halogenated phenylalanine derivatives.

Amino acid analogues having detectable labels are also usefully incorporated during synthesis to provide a labeled polypeptide. Biotin, for example can be added using biotinoyl-(9-fluorenylmethoxycarbonyl)-L-lysine (FMOC biocytin) (Molecular Probes, Eugene, Oreg., USA). (Biotin can also be added enzymatically by incorporation into a fusion protein of a E. coli BirA substrate peptide.) The FMOC and tBOC derivatives of dabcyl-L-lysine (Molecular Probes, Inc., Eugene, Oreg., USA) can be used to incorporate the dabcyl chromophore at selected sites in the peptide sequence during synthesis. The aminonaphthalene derivative EDANS, the most common fluorophore for pairing with the dabcyl quencher in fluorescence resonance energy transfer (FRET) systems, can be introduced during automated synthesis of peptides by using EDANS—FMOC-L-glutamic acid or the corresponding tBOC derivative (both from Molecular Probes, Inc., Eugene, Oreg., USA). Tetramethylrhodamine fluorophores can be incorporated during automated FMOC synthesis of peptides using (FMOC)—TMR-L-lysine (Molecular Probes, Inc. Eugene, Oreg., USA).

Other useful amino acid analogues that can be incorporated during chemical synthesis include aspartic acid, glutamic acid, lysine, and tyrosine analogues having allyl side-chain protection (Applied Biosystems, Inc., Foster City, Calif., USA); the allyl side chain permits synthesis of cyclic, branched-chain, sulfonated, glycosylated, and phosphorylated peptides. A large number of other FMOC-protected non-natural amino acid analogues capable of incorporation during chemical synthesis are available commercially, e.g., from The Peptide Laboratory (Richmond, Calif., USA).

Non-natural amino acid residues can also be added biosynthetically by engineering a suppressor tRNA, typically one that recognizes the UAG stop codon, by chemical aminoacylation with the desired unnatural amino acid and. Conventional site-directed mutagenesis is used to introduce the chosen stop codon UAG at the site of interest in the protein gene. When the acylated suppressor tRNA and the mutant gene are combined in an in vitro transcription/translation system, the unnatural amino acid is incorporated in response to the UAG codon to give a protein containing that amino acid at the specified position. Liu et al., Proc. Natl Acad. Sci. USA 96(9):4780-5 (1999); Wang et al., Science 292(5516):498-500 (2001).

The isolated GP354 proteins, protein fragments and fusion proteins of the present invention can also include non-native inter-residue bonds, including bonds that lead to circular and branched forms. The isolated GP354 proteins and protein fragments of the present invention can also include post-translational and post-synthetic modifications, either throughout the length of the protein or localized to one or more portions thereof.

For example, when produced by recombinant expression in eukaryotic cells, the isolated proteins, fragments, and fusion proteins of the present invention will typically include N-linked and/or 0-linked glycosylation, the pattern of which will reflect both the availability of glycosylation sites on the protein sequence and the identity of the host cell. Further modification of glycosylation pattern can be performed enzymatically. As another example, recombinant polypeptides of the invention may also include an initial modified methionine residue, in some cases resulting from host-mediated processes.

When the proteins, protein fragments, and protein fusions of the present invention are produced by chemical synthesis, post-synthetic modification can be performed before deprotection and cleavage from the resin or after deprotection and cleavage. Modification before deprotection and cleavage of the synthesized protein often allows greater control, e.g. by allowing targeting of the modifying moiety to the N-terminus of a resin-bound synthetic peptide. Useful post-synthetic (and post-translational) modifications include conjugation to detectable labels, such as fluorophores. A wide variety of amine-reactive and thiol-reactive fluorophore derivatives have been synthesized that react under nondenaturing conditions with N-terminal amino groups and epsilon amino groups of lysine residues, on the one hand, and with free thiol groups of cysteine residues, on the other.

Kits are available commercially that permit conjugation of proteins to a variety of amine-reactive or thiol-reactive fluorophores: Molecular Probes, Inc. (Eugene, Oreg., USA), e.g., offers kits for conjugating proteins to Alexa Fluor 350, Alexa Fluor 430, Fluorescein-EX, Alexa Fluor 488, Oregon Green 488, Alexa Fluor 532, Alexa Fluor 546, Alexa Fluor 546, Alexa Fluor 568, Alexa Fluor 594, and Texas Red-X. A wide variety of other amine-reactive and thiol-reactive fluorophores are available commercially (Molecular Probes, Inc., Eugene, Oreg., USA), including Alexa Fluor® 350, Alexa Fluor® 488, Alexa Fluor® 532, Alexa Fluor® 546, Alexa Fluor® 568, Alexa Fluor® 594, Alexa Fluo® 647 (monoclonal antibody labeling kits), BODIPY dyes, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red.

The polypeptides of the present invention can also be conjugated to fluorophores, other proteins, and other macromolecules, using bifunctional linking reagents. Common homobifunctional reagents include, e.g., APG, AEDP, BASED, BMB, BMDB, BMH, BMOE, BM[PEO]3, BM[PEO]4, BS3, BSOCOES, DFDNB, DMA, DMP, DMS, DPDPB, DSG, DSP (Lomant's Reagent), DSS, DST, DTBP, DTME, DTSSP, EGS, HBVS, Sulfo-BSOCOES, Sulfo-DST, Sulfo-EGS (all available from Pierce, Rockford, Ill., USA); common heterobifunctional cross-linkers include ABH, AMAS, ANB-NOS, APDP, ASBA, BMPA, BMPH, BMPS, EDC, EMCA, EMCH, EMCS, KMUA, KMUH, GMBS, LC-SMCC, LC-SPDP, MBS, M2C2H, MPBH, MSA, NHS-ASA, PDPH, PMPI, SADP, SAED, SAND, SANPAH, SASD, SATP, SBAP, SFAD, SIA, SIAB, SMCC, SMPB, SMPH, SMPT, SPDP, Sulfo-EMCS, Sulfo-GMBS, Sulfo-HSAB, Sulfo-KMUS, Sulfo-LC-SPDP, Sulfo-MBS, Sulfo-NHS-LC-ASA, Sulfo-SADP, Sulfo-SANPAH, Sulfo-SIAB, Sulfo-SMCC, Sulfo-SMPB, Sulfo-LC-SMPT, SVSB, TFCS (all available Pierce, Rockford, Ill., USA).

The proteins, protein fragments, and protein fusions of the present invention can be conjugated, using such cross-linking reagents, to fluorophores that are not amine- or thiol-reactive. Other labels that usefully can be conjugated to the proteins, protein fragments, and fusion proteins of the present invention include radioactive labels, echosonographic contrast reagents, and MRI contrast agents. The proteins, protein fragments, and protein fusions of the present invention can also usefully be conjugated using cross-linking agents to carrier proteins, such as KLH, bovine thyroglobulin, and even bovine serum albumin (BSA), to increase immunogenicity for raising anti-GP354 antibodies.

The GP354 proteins, protein fragments, and protein fusions of the present invention can also usefully be conjugated to polyethylene glycol (PEG); PEGylation increases the serum half life of proteins administered intravenously for replacement therapy. Delgado et al., Crit. Rev. Ther. Drug Carrier Syst. 9(3-4):249-304 (1992); Scott et al., Curr. Pharm. Des. 4(6):423-38 (1998); DeSantis et al., Curr. Opin. Biotechnol. 10(4):324-30 (1999), incorporated herein by reference in their entireties. PEG monomers can be attached to the protein directly or through a linker, with PEGylation using PEG monomers activated with tresyl chloride (2,2,2-trifluoroethanesulphonyl chloride) permitting direct attachment under mild conditions.

The isolated GP354 proteins of the present invention, including fusions thereof, can be produced by recombinant expression, typically using the expression vectors of the present invention as above-described or, especially if fewer than about 100 amino acids, optionally by chemical synthesis (typically, solid phase synthesis), and, on occasion, by in vitro translation.

Production of the isolated proteins of the present invention can optionally be followed by purification. Purification of recombinantly expressed proteins is now well within the skill in the art. See, e.g., Thomer et al. (eds.), Applications of Chimeric Genes and Hybrid Proteins, Part A: Gene Expression and Protein Purification (Methods in Enzymology, Volume 326), Academic Press (2000), (ISBN: 0121822273); Harbin (ed.), Cloning, Gene Exression and Protein Purification: Experimental Procedures and Process Rationale, Oxford Univ. Press (2001) (ISBN: 0195132947); Marshak et al., Strategies for Protein Purification and Characterization: A Laboratory Course Manual, Cold Spring Harbor Laboratory Press (1996) (ISBN: 0-87969-385-1); and Roe (ed.), Protein Purification Applications, Oxford University Press (2001), the disclosures of which are incorporated herein by reference in their entireties, and thus need not be detailed here.

Briefly, however, if purification tags have been fused through use of an expression vector that appends such tag, purification can be effected, at least in part, by means appropriate to the tag, such as use of immobilized metal affinity chromatography for polyhistidine tags. Other techniques common in the art include ammonium sulfate fractionation, immuno-precipitation, fast protein liquid chromatography (FPLC), high performance liquid chromatography (HPLC), and preparative gel electrophoresis. Purification of chemically-synthesized peptides can readily be effected, e.g., by HPLC.

Accordingly, it is an aspect of the present invention to provide the isolated GP354 proteins of the present invention in pure or substantially pure form. A purified protein of the present invention is an isolated protein, as above described, that is present at a concentration of at least 95%, as measured on a mass basis (w/w) with respect to total protein in a composition. Such purities can often be obtained during chemical synthesis without further purification, as, e.g., by HPLC. Purified proteins of the present invention can be present at a concentration (measured on a mass basis with respect to total protein in a composition) of 96%, 97%, 98%, and even 99%. The proteins of the present invention can even be present at levels of 99.5%, 99.6%, and even 99.7%, 99.8%, or even 99.9% following purification, as by HPLC.

Although high levels of purity are preferred when the isolated proteins of the present invention are used as therapeutic agents—such as vaccines, or for replacement therapy—the isolated proteins of the present invention are also useful at lower purity. For example, partially purified proteins of the present invention can be used as immunogens to raise antibodies in laboratory animals.

Thus, the present invention provides the isolated proteins of the present invention in substantially purified form. A “substantially purified protein” of the present invention is an isolated protein, as above described, present at a concentration of at least 70%, measured on a mass basis with respect to total protein in a composition. Usefully, the substantially purified protein is present at a concentration, measured on a mass basis with respect to total protein in a composition, of at least 75%, 80%, or even at least 85%, 90%, 91%, 92%, 93%, 94%, 94.5% or even at least 94.9%.

In preferred embodiments, the purified and substantially purified proteins of the present invention are in compositions that lack detectable ampholytes, acrylamide monomers, bis-acrylamide monomers, and polyacrylamide.

The GP354 proteins, fragments, and fusions of the present invention can usefully be attached to a substrate. The substrate can porous, substantially nonporous (such as plastic), or solid; planar or non-planar; the bond can be covalent or noncovalent. Porous substrates, commonly membranes, typically comprise nitrocellulose, polyvinylidene fluoride (PVDF), or cationically derivatized, hydrophilic PVDF; so bound, the proteins, fragments, and fusions of the present invention can be used to detect and quantify antibodies, e.g. in serum, that bind specifically to the immobilized protein of the present invention. Proteins, fragments, and fusions of the present invention when bound to substantially nonporous substrates, such as plastics, may be used to detect and quantify antibodies, e.g. in serum, that bind specifically to the immobilized protein of the present invention.

The proteins, fragments, and fusions of the present invention can also be attached to a substrate suitable for use as a surface enhanced laser desorption ionization source; so attached, the protein, fragment, or fusion of the present invention is useful for binding and then detecting secondary proteins that bind with sufficient affinity or avidity to the surface-bound protein to indicate biologic interaction therebetween.

The proteins, fragments, and fusions of the present invention can also be attached to a substrate suitable for use in surface plasmon resonance detection. So attached, the protein, fragment, or fusion of the present invention is useful for binding and then detecting secondary proteins that bind with sufficient affinity or avidity to the surface-bound protein to indicate significant biological interaction between the two.

### Antibodies and Antibody-producing Cells

The invention provides antibodies, including fragments and derivatives thereof, that bind specifically to GP354 proteins and protein fragments of the invention, or that bind to one or more of the proteins and protein fragments encoded by the isolated GP354 nucleic acids of the invention. The antibodies can be specific for linear epitopes, discontinuous epitopes, or conformational epitopes of such proteins or protein fragments, either as present on the protein in its native conformation or, in some cases, as present on the proteins as denatured, as, e.g., by solubilization in SDS.

The invention also provides antibodies, including fragments and derivatives thereof, the binding of which can be competitively inhibited by one or more of the GP354 proteins and protein fragments of the present invention, or by one or more of the proteins and protein fragments encoded by the isolated gp354 polynucleotides of the present invention.

In a first series of antibody embodiments, the invention provides antibodies, both polyclonal and monoclonal, and fragments and derivatives thereof, that bind specifically to a polypeptide having an amino acid sequence presented in SEQ ID NO:2, 4, 8, 10 or 12.

Such antibodies are useful in a variety of in vitro immunoassays, such as Western blotting and ELISA. Such antibodies are also useful in isolating and purifying GP354 proteins, including related cross-reactive proteins, by immuno-precipitation, immunoaffinity chromatography, or magnetic bead-mediated purification. Such methods are well-known in the art.

In a second series of antibody embodiments, the invention provides antibodies, both polyclonal and monoclonal, and fragments and derivatives thereof, the specific binding of which can be competitively inhibited by the isolated proteins and polypeptides of the present invention.

In other embodiments, the invention further provides the above-described antibodies detectably labeled, and in yet other embodiments, provides the above-described antibodies attached to a substrate.

Anti-PanCAM antibodies (Examples 11 and 19, infra) of this invention can be used to isolate pancreatic beta cells (e.g., from large animals such as cows, pigs, and horses; or from primates such as monkeys and chimpanzees). Beta cells so obtained are useful in making limited-lifetime encapsulated (and therefore immune-protected) xenografts in diabetic patients (such as human patients).

In some embodiments, the antibodies of this invention are used to target therapeutic agents to beta islets. For instance, type I diabetes is marked by T cell infiltration into beta islets. Thus, cytotoxic agents (i.e., agents that impair the viability and/or the functions of a cell) specific for these T cells (e.g., anti-CD3, -CD4, or -CD8 antibodies) can be conjugated to anti-PanCAM antibodies and thereby directed to the diseased site. In another example, antisense therapeutics (such as antisense oligos to MHC class II molecules, which are highly expressed in T lymphocytes attacking beta cells; Chaturvedi et al., J Immunol 164(12):6610-20 (2000)) can be linked to anti-PanCAM antibodies and be directed to the beta islets.

The antibodies of this invention can also be conjugated to cytotoxic agents (e.g., nonspecific agents such as radioactive agents; targetable toxins such as ricin and cholera toxin; and chemotherapy agents such as doxorubicin) so as to direct the cytotoxins to the site of insulinomas, which are difficult to treat because they tend to be multi-focal. The antibodies may also be used to deliver DNA to the insulinoma as gene therapy to inhibit or otherwise modify the behavior of the tumor (e.g., to deliver an antisense oligonucleotide or DNA construct to either the responsible oncogene or the insulin gene itself, or to a gene in the signal transduction machinery regulating insulin release).

As used herein, the term “antibody” refers to a polypeptide, at least a portion of which is encoded by at least one immunoglobulin gene, which can bind specifically to a first molecular species, and to fragments or derivatives thereof that remain capable of such specific binding.

By “bind specifically” and “specific binding” is here intended the ability of the antibody to bind to a first molecular species in preference to binding to other molecular species with which the antibody and first molecular species are admixed. An antibody is said specifically to “recognize” a first molecular species when it can bind specifically to that first molecular species.

As is well known in the art, the degree to which an antibody can discriminate as among molecular species in a mixture will depend, in part, upon the conformational relatedness of the species in the mixture; typically, the antibodies of the present invention will discriminate over adventitious binding to non-GP354 proteins by at least two-fold, more typically by at least 5-fold, typically by more than 10-fold, 25-fold, 50-fold, 75-fold, and often by more than 100-fold, and on occasion by more than 500-fold or 1000-fold. When used to detect the proteins or protein fragments of the present invention, the antibody of the present invention is sufficiently specific when it can be used to determine the presence of the protein of the present invention in samples derived from human pancreatic and neural tissues.

Typically, the affinity or avidity of an antibody (or antibody multimer, as in the case of an IgM pentamer) of the present invention for a GP354 protein or protein fragment of the present invention will be at least about 1×10⁻⁶ molar (M), typically at least about 5×10⁻⁷ M, usefully at least about 1×10⁻⁷ M, with affinities and avidities of at least 1×10⁻⁸ M, 5×10⁻⁹ M, and 1×10⁻¹⁰ M proving especially useful.

The antibodies of the present invention can be naturally-occurring forms, such as IgG, IgM, IgD, IgE, and IgA, from any mammalian species.

Human antibodies can, but will infrequently, be drawn directly from human donors or human cells. In such case, antibodies to the proteins of the present invention will typically have resulted from fortuitous immunization, such as autoimmune immunization, with the protein or protein fragments of the present invention. Such antibodies will typically, but will not invariably, be polyclonal.

Human antibodies are more frequently obtained using transgenic animals that express human immunoglobulin genes, which transgenic animals can be affirmatively immunized with a GP354 protein immunogen of the present invention. Human Ig-transgenic mice capable of producing human antibodies and methods of producing human antibodies therefrom upon specific immunization are well known in the art. See, e.g., in U.S. Pat. Nos. 6,162,963; 6,150,584; 6,114,598; 6,075,181; 5,939,598; 5,877,397; 5,874,299; 5,814,318; 5,789,650; 5,770,429; 5,661,016; 5,633,425; 5,625,126; 5,569,825; 5,545,807; 5,545,806, and 5,591,669, the disclosures of which are incorporated herein by reference in their entireties. Such antibodies are typically monoclonal, and are typically produced using techniques developed for production of murine antibodies.

Human antibodies are particularly useful, and often preferred, when the antibodies of the present invention are to be administered to human beings as in vivo diagnostic or therapeutic agents, since recipient immune response to the administered antibody will often be substantially less than that occasioned by administration of an antibody derived from another species, such as mouse.

IgG, IgM, IgD, IgE and IgA antibodies of the present invention are also usefully obtained from other mammalian species, including rodents—typically mouse, but also rat, guinea pig, and hamster—lagomorphs, typically rabbits, and also larger mammals, such as sheep, goats, cows, and horses. In such cases, as with the transgenic human-antibody-producing non-human mammals, fortuitous immunization is not required, and the non-human mammal is typically affirmatively immunized, according to standard immunization protocols, with the protein or protein fragment of the present invention.

As discussed above, virtually all fragments of eight or more contiguous amino acids of the proteins of the present invention can be used effectively as immunogens when conjugated to a carrier, typically a protein such as bovine thyroglobulin, keyhole limpet hemocyanin, or bovine serum albumin, conveniently using a bifunctional linker such as those described elsewhere above, which discussion is incorporated by reference here.

Immunogenicity can also be conferred by fusion of the proteins and protein fragments of the present invention to other moieties. Peptides of the present invention can, for example, be produced by solid phase synthesis on a branched polylysine core matrix; these multiple antigenic peptides (MAPs) provide high purity, increased avidity, accurate chemical definition and improved safety in vaccine development. Tam et al., Proc. Natl. Acad. Sci. USA 85:5409-5413 (1988); Posnett et al., J. Biol. Chem. 263, 1719-1725 (1988).

Protocols for immunizing non-human mammals are well-established in the art, Harlow et al. (eds.), Antibodies: A Laboratory Manual, Cold Spring Harbor Laboratory (1998) (ISBN: 0879693142); Coligan et al. (eds.), Current Protocols in Immunology, John Wiley & Sons, Inc. (2001) (ISBN: 0-471-52276-7); Zola, Monoclonal Antibodies: Preparation and Use of Monoclonal Antibodies and Engineered Antibody Derivatives (Basics: From Background to Bench), Springer Verlag (2000) (ISBN: 0387915907), the disclosures of which are incorporated herein by reference.

Antibodies from nonhuman mammals can be polyclonal or monoclonal, with polyclonal antibodies having certain advantages in immuno-histochemical detection of the proteins of the present invention and monoclonal antibodies having advantages in identifying and distinguishing particular epitopes of the proteins of the present invention.

Following immunization, the antibodies of the present invention can be produced using any art-accepted technique. Such techniques are well known in the art, Coligan et al. (eds.), Current Protocols in Immunology, John Wiley & Sons, Inc. (2001) (ISBN: 0-471-52276-7); Zola, Monoclonal Antibodies: Preparation and Use of Monoclonal Antibodies and Engineered Antibody Derivatives (Basics: From Background to Bench), Springer Verlag (2000) (ISBN: 0387915907); Howard et al. (eds.), Basic Methods in Antibody Production and Characterization, CRC Press (2000) (ISBN: 0849394457); Harlow et al. (eds.), Antibodies: A Laboratory Manual, Cold Spring Harbor Laboratory (1998) (ISBN: 0879693142); Davis (ed.), Monoclonal Antibody Protocols, Vol. 45, Humana Press (1995) (ISBN: 0896033082); Delves (ed.), Antibody Production: Essential Techniques, John Wiley & Son Ltd (1997) (ISBN: 0471970107); Kenney, Antibody Solution: An Antibody Methods Manual, Chapman & Hall (1997) (ISBN: 0412141914), incorporated herein by reference in their entireties, and thus need not be detailed here.

Recombinant expression in host cells is particularly useful when fragments or derivatives of the antibodies of the present invention are desired. Host cells for recombinant antibody production—either whole antibodies, antibody fragments, or antibody derivatives—can be prokaryotic or eukaryotic.

Prokaryotic hosts are particularly useful for producing phage displayed antibodies of the present invention. The technology of phage-displayed antibodies, in which antibody variable region fragments are fused, for example, to the gene III protein (pIII) or gene VIII protein (pVIII) for display on the surface of filamentous phage, such as M13, is by now well-established, Sidhu, Curr. Opin. Biotechnol. 11(6):610-6 (2000); Griffiths et al., Curr. Opin. Biotechnol. 9(1): 102-8 (1998); Hoogenboom et al., Immunotechnology, 4(1):1-20 (1998); Rader et al., Curr. Opin. Biotechnol. 8:503-508 (1997); Aujame et al., Hum. Antibodies 8:155-168 (1997); Hoogenboom, Trends Biotechnol. 15:62-70 (1997); de Kruif et al., 17:453-455 (1996); Barbas et al., Trends Biotechnol. 14:230-234 (1996); Winter et al., Annu. Rev. Immunol. 433-455 (1994), and techniques and protocols required to generate, propagate, screen (pan), and use the antibody fragments from such libraries have recently been compiled, Barbas et al., Phage Display: A Laboratory Manual, Cold Spring Harbor Laboratory Press (2001) (ISBN 0-87969-546-3); Kay et al. (eds.), Phage Display of Peptides and Proteins: A Laboratory Manual, Academic Press, Inc. (1996); Abelson et al. (eds.), Combinatorial Chemistry, Methods in Enzymology vol. 267, Academic Press (May 1996), the disclosures of which are incorporated herein by reference in their entireties. Typically, phage-displayed antibody fragments are scFv fragments or Fab fragments; when desired, full length antibodies can be produced by cloning the variable regions from the displaying phage into a complete antibody and expressing the full length antibody in a further prokaryotic or a eukaryotic host cell.

Eukaryotic cells are also useful for expression of the antibodies, antibody fragments, and antibody derivatives of the present invention. For example, antibody fragments of the present invention can be produced in Pichia pastoris, Takahashi et al., Biosci. Biotechnol. Biochem. 64(10):2138-44 (2000); Freyre et al., J. Biotechnol. 76(2-3):157-63 (2000); Fischer et al., Biotechnol. Appl. Biochem. 30 (Pt 2):117-20 (1999); Pennell et al., Res. Immunol. 149(6):599-603 (1998); Eldin et al., J. Immunol. Methods. 201(1):67-75 (1997); and in Saccharomyces cerevisiae, Frenken et al., Res. Immunol. 149(6):589-99 (1998); Shusta et al., Nature Biotechnol. 16(8):773-7 (1998), the disclosures of which are incorporated herein by reference in their entireties.

Antibodies, including antibody fragments and derivatives, of the invention can also be produced in insect cells, Li et al., Protein Expr. Purif 21(l):121-8 (2001); Ailor et al., Biotechnol. Bioeng. 58(2-3):196-203 (1998); Hsu et al., Biotechnol. Prog. 13(1):96-104 (1997); Edelman et al., Immunology 91(1):13-9 (1997); and Nesbit et al., J. Immunol. Methods. 151(1-2):201-8 (1992), the disclosures of which are incorporated herein by reference in their entireties.

Antibodies and fragments and derivatives thereof of the present invention may also be produced in plant cells, Giddings et al., Nature Biotechnol. 18(11): 1151-5 (2000); Gavilondo et al., Biotechniques 29(1):128-38 (2000); Fischer et al., J. Biol. Regul. Homeost. Agents 14(2):83-92 (2000); Fischer et al., Biotechnol. Appl. Biochem. 30 (Pt 2):113-6 (1999); Fischer et al., Biol. Chem. 380(7-8):825-39 (1999); Russell, Curr. Top. Microbiol. Immunol. 240:119-38 (1999); and Ma et al., Plant Physiol. 109(2):341-6 (1995), the disclosures of which are incorporated herein by reference in their entireties.

Mammalian cells useful for recombinant expression of antibodies, antibody fragments, and antibody derivatives of the present invention include CHO cells, COS cells, 293 cells, and myeloma cells. Verma et al., J. Immunol. Methods 216(1-2): 165-81 (1998), review and compare bacterial, yeast, insect and mammalian expression systems for expression of antibodies.

Antibodies of the present invention may also be prepared by cell free translation, as further described in Merk et al., J. Biochem. (Tokyo). 125(2):328-33 (1999) and Ryabova et al., Nature Biotechnol. 15(1):79-84 (1997), and in the milk of transgenic animals, as further described in Pollock et al., J. Immunol. Methods 231(1-2):147-57 (1999), the disclosures of which are incorporated herein by reference in their entireties.

The invention further provides antibody fragments that bind specifically to one or more of the GP354 proteins and protein fragments of the present invention, to one or more of the proteins and protein fragments encoded by the isolated gp354 polynucleotides of the present invention, or the binding of which can be competitively inhibited by one or more of the proteins and protein fragments of the present invention or one or more of the proteins and protein fragments encoded by the isolated polynucleotides of the present invention.

Among such useful fragments are Fab, Fab′, Fv, F(ab)′₂, and single chain Fv (scFv) fragments. Other useful fragments are described in Hudson, Curr. Opin. Biotechnol. 9(4):395-402 (1998). The present invention thus provides antibody derivatives that bind specifically to one or more of the GP354 proteins and protein fragments of the present invention, to one or more of the proteins and protein fragments encoded by the isolated nucleic acids of the present invention, or the binding of which can be competitively inhibited by one or more of the proteins and protein fragments of the present invention or one or more of the proteins and protein fragments encoded by the isolated polynucleotides of the present invention.

Among such useful derivatives are chimeric, primatized, and humanized antibodies; such derivatives are less immunogenic in human beings, and thus more suitable for in vivo administration, than are unmodified antibodies from non-human mammalian species.

Chimeric antibodies typically include heavy and/or light chain variable regions (including both CDR and framework residues) of immunoglobulins of one species, typically mouse, fused to constant regions of another species, typically human. See, e.g., U.S. Pat. No. 5,807,715; Morrison et al., Proc. Natl. Acad. Sci USA.81(21):6851-5 (1984); Sharon et al., Nature 309(5966):364-7 (1984); Takeda et al., Nature 314(6010):452-4 (1985), the disclosures of which are incorporated herein by reference in their entireties.

Primatized and humanized antibodies typically include heavy and/or light chain CDRs from a murine antibody grafted into a non-human primate or human antibody V region framework, usually further comprising a human constant region, Riechmann et al., Nature 332(6162):323-7 (1988); Co et al., Nature 351(6326):501-2 (1991); U.S. Pat. Nos. 6,054,297; 5,821,337; 5,770,196; 5,766,886; 5,821,123; 5,869,619; 6,180,377; 6,013,256; 5,693,761; and 6,180,370, the disclosures of which are incorporated herein by reference in their entireties.

Other useful antibody derivatives of the invention include heteromeric antibody complexes and antibody fusions, such as diabodies (bispecific antibodies), single-chain diabodies, and intrabodies.

The antibodies of the present invention, including fragments and derivatives thereof, can usefully be labeled. It is, therefore, another aspect of the present invention to provide labeled antibodies that bind specifically to one or more of the proteins and protein fragments of the present invention, to one or more of the GP354 proteins and protein fragments encoded by the isolated polynucleotides of the present invention, or the binding of which can be competitively inhibited by one or more of the proteins and protein fragments of the present invention or one or more of the proteins and protein fragments encoded by the isolated polynucleotides of the present invention.

The choice of label depends, in part, upon the desired use. When the antibodies of the present invention are used for immunohistochemical staining of tissue samples, the label can usefully be an enzyme that catalyzes production and local deposition of a detectable product. Enzymes typically conjugated to antibodies to permit their immunohistochemical visualization are well known, and include alkaline phosphatase, β-galactosidase, glucose oxidase, horseradish peroxidase (HRP), and urease. The antibodies of the invention can also be labeled using colloidal gold.

A multitude of typical substrates for production and deposition of visually detectable products, luminescent and fluorescent labels, are also well known and need not be further described. See, e.g., Thorpe et al., Methods Enzymol. 133:331-53 (1986); Kricka et al., J. Immunoassay 17(1):67-83 (1996); and Lundqvist et al., J. Biolumin. Chemilumin. 10(6):353-9 (1995), the disclosures of which are incorporated herein by reference in their entireties. Kits for enhanced chemiluminescent detection (ECL) are available commercially.

When the antibodies of the present invention are used, e.g., for flow cytometric detection, for scanning laser cytometric detection, or for fluorescent immunoassay, they can usefully be labeled with fluorophores. There are a wide variety of fluorophore labels that can usefully be attached to the antibodies of the present invention. Many are available, e.g., from Molecular Probes, Inc., Eugene, Oreg., USA.

For secondary detection using labeled avidin, streptavidin, captavidin or neutravidin, the antibodies of the present invention can usefully be labeled with biotin.

When the antibodies of the present invention are used, e.g., for Western blotting applications, they can usefully be labeled with radioisotopes, such as ³³P, ³²P, ³⁵S, ³H, and ¹²⁵I. As another example, when the antibodies of the present invention are used for radioimmunotherapy, the label can usefully be ²²⁸Th, ²²⁷Ac, ²²⁵Ac, ²²³Ra, ²¹³B, ²¹²pb, ²¹²Bi,²¹¹ At, ²⁰³Pb, ¹⁹⁴Os, ¹⁸⁸Re, ¹⁸⁶Re, ¹⁵³Sm, ¹⁴⁹Tb, ¹³¹I, ¹²⁵I, ¹¹¹In, ¹⁰⁵Rh, ^{99m}Tc, ⁹⁷Ru, ⁹⁰Y ⁹⁰Sr, ⁸⁸Y, ⁷²Se, ⁶⁷Cu, or ⁴⁷Sc. As another when the antibodies of the present invention are to be used for in vivo diagnostic use, they can be rendered detectable by conjugation to MRI contrast agents, such as gadolinium diethylenetriaminepentaacetic acid (DTPA), Lauffer et al., Radiology 207(2):529-38 (1998), or by radioisotopic labeling. As would be understood by the skilled artisan, use of any of the labels described above is not restricted to the application as for which they were mentioned.

The antibodies of the present invention, including fragments and derivatives thereof, can also be conjugated to biologically deleterious moieties, such as toxins, in order to target the toxin's ablative action to cells that display and/or express the proteins of the present invention. Commonly, the antibody in such immunotoxins is conjugated to Pseudomonas exotoxin A, diphtheria toxin, shiga toxin A, anthrax toxin lethal factor, or ricin. See Hall (ed.), Immunotoxin Methods and Protocols (Methods in Molecular Biology, Vol 166), Humana Press (2000) (ISBN:0896037754); and Frankel et al. (eds.), Clinical Applications of Immunotoxins, Springer-Verlag New York, Incorporated (1998) (ISBN:3540640975), the disclosures of which are incorporated herein by reference in their entireties, for review.

The antibodies of the present invention can usefully be attached to a substrate. The invention thus provides antibodies that bind specifically to one or more of the GP354 proteins and protein fragments of the present invention, to one or more of the proteins and protein fragments encoded by the isolated polynucleotides of the present invention, or the binding of which can be competitively inhibited by one or more of the proteins and protein fragments of the present invention or one or more of the proteins and protein fragments encoded by the isolated polynucleotides of the present invention, attached to a substrate. Substrates can be porous or nonporous, planar or nonplanar.

For example, the antibodies of the present invention can usefully be conjugated to filtration media, such as NHS-activated Sepharose or CNBr-activated Sepharose for purposes of immunoaffinity chromatography.

The antibodies of the present invention can also usefully be attached to paramagnetic microspheres, typically by biotin-streptavidin interaction, which microsphere can then be used for isolation of cells that express or display the proteins of the present invention. As another example, the antibodies of the present invention can usefully be attached to the surface of a microtiter plate for ELISA.

As noted above, the antibodies of the present invention can be produced in prokaryotic and eukaryotic cells. The invention thus also provides cells that express the antibodies of the present invention, including hybridoma cells, B cells, plasma cells, and host cells recombinantly modified to express the antibodies of the present invention.

The present invention also provides aptamers evolved to bind specifically to one or more of the GP354 proteins and protein fragments of the present invention, to one or more of the proteins and protein fragments encoded by the isolated polynucleotides of the present invention, or the binding of which can be competitively inhibited by one or more of the proteins and protein fragments of the present invention or one or more of the proteins and protein fragments encoded by the isolated polynucleotides of the present invention.

### Pharmaceutical Compositions and Therapeutic Methods

GP354 is a new member of the immunoglobulin (Ig) superfamily expressed predominantly in the pancreas and in lower amounts in neural tissue, e.g., the CNS. GP354, an integral cell surface membrane protein, has five signature Ig domains in its extracellular portion which are known in other family members to mediate cell-cell recognition and adhesion reactions. As a member of the Ig superfamily, GP354 is likely important for mediating cell-cell recognition, binding and adhesion functions in the pancreatic, neural and potentially other tissues in which it is expressed.

As a pancreatic enriched protein, GP354 will be a suitable therapeutic target for treating abnormal conditions, disorders and/or diseases related to improper cell-cell binding, adhesion and signaling in the pancreas, particularly during tissue development and during tissue regeneration and/or healing, e.g., after pancreatic damage, trauma or degenerative conditions. It is also envisioned that GP354 will be useful for inhibiting pancreatic cell death associated with immune, auto-immune, and degenerative conditions. It is envisioned that the neural form of GP354 will be a similarly suitable therapeutic target for tissue regeneration and repair and for inhibiting degeneration and cell death in CNS tissue.

The invention accordingly provides pharmaceutical compositions comprising nucleic acids, proteins, and antibodies of the present invention, as well as mimetics, agonists, antagonists, or modulators of GP354 activity, may be administered as pharmaceutical agents for the treatment (i.e., the amelioration of) of disorders, conditions or diseases associated with mis-expression of GP354 or to overcome abnormal expression or activities of other components which participate in GP354 related molecular and cellular recognition pathways. As GP354 expression is relatively concentrated in the pancreas, it is anticipated that GP354 mis-expression may be associated with pancreatic disorder or disease, and/or with congenital defects in pancreatic development of function.

Disorders and diseases of the pancreas, for which administration of a composition of the invention may be useful, include acute pancreatitis (often but not always manifesting in abnormal pancreatic exocrine functions, such as elevated serum, ascitic and/or pleural fluid amylase levels, or abnormal lipase or trypsinogen levels. Pancreatic inflammation and necrosis are also associated with acute as well as with chronic pancreatitis and exocrine insufficiency. A variety of pancreatic endocrine tumors have been characterized, and auto-immune disorders which affect the pancreas have also been described. For a more detailed description of diagnoses and treaments of pancreatic disorders and diseases, see Harrison's PRINCIPLES OF INTERNAL MEDICINE, 14^{th} Ed., (Anthony S. Fauci et al., editors), McGraw-Hill Companies, Inc., 1998, Part Eleven, Section 3, the disclosure of which is incorporated by reference in its entirety.

GP354 expression is also detected in neural CNS tissue, albeit at lower levels than is detected in the pancreas. It is therefore envisioned that GP354 mis-expression may be associated with neural dysfunction, disorder or disease, or abnormal development of the CNS. Examples of neural disorders which may be ameliorated by treatment with a composition of the invention include, without limitation, Alzheimer's disease, Parkinson's disease, senile dementia, migraine, epilepsy, neuritis, neurasthenia, neuropathy, and any other diseases involving GP354-mediated neural migration, neural degeneration (e.g., GP354-mediated autoimmune diseases such as certain forms of multiple sclerosis), and neural tumors (e.g., glioma, astroblastoma, and astrocytoma).

Some other diseases for which compositions of the invention may have utility include endocrine and hormonal problems (e.g., diabetes), pancreatic diseases, cancers (particularly pancreatic cancer), and the like. The use of GP354 modulators, including GP354 antisense reagents, GP354 ligands and anti-GP354 antibodies, to treat individuals having or at risk of developing such diseases is an aspect of the invention.

A composition of the invention typically contains from about 0.1 to 90% by weight (such as 1 to 20% or 1 to 10%) of a therapeutic agent of the invention in a pharmaceutically accepted carrier. Solid formulations of the compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, or alginic acid. Disintegrators that can be used include, without limitation, microcrystalline cellulose, corn starch, sodium starch glycolate, and alginic acid. Tablet binders that can be used include acacia, methylcellulose, sodium carboxymethylcellulose, polyvinylpyrrolidone(Povidone™), hydroxypropyl methylcellulose, sucrose, starch, and ethylcellulose. Lubricants that can be used include magnesium stearates, stearic acid, silicone fluid, talc, waxes, oils, and colloidal silica.

Liquid formulations of the compositions for oral administration prepared in water or other aqueous vehicles can contain various suspending agents such as methylcellulose, alginates, tragacanth, pectin, kelgin, carrageenan, acacia, polyvinylpyrrolidone, and polyvinyl alcohol. The liquid formulations can also include solutions, emulsions, syrups and elixirs containing, together with the active compound(s), wetting agents, sweeteners, and coloring and flavoring agents. Various liquid and powder formulations can be prepared by conventional methods for inhalation into the lungs of the mammal to be treated.

Injectable formulations of the compositions can contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol, and the like). For intravenous injections, water soluble versions of the compounds can be administered by the drip method, whereby a pharmaceutical formulation containing the antifungal agent and a physiologically acceptable excipient is infused. Physiologically acceptable excipients can include, for example, 5% dextrose, 0.9% saline, Ringer's solution or other suitable excipients. Intramuscular preparations, e.g., a sterile formulation of a suitable soluble salt form of the compounds, can be dissolved and administered in a pharmaceutical excipient such as Water-for-Injection, 0.9% saline, or 5% glucose solution. A suitable insoluble form of the compound can be prepared and administered as a suspension in an aqueous base or a pharmaceutically acceptable oil base, such as an ester of a long chain fatty acid (e.g., ethyl oleate).

A topical semi-solid ointment formulation typically contains a concentration of the active ingredient from about 1 to 20%, e.g., 5 to 10%, in a carrier such as a pharmaceutical cream base. Various formulations for topical use include drops, tinctures, lotions, creams, solutions, and ointments containing the active ingredient and various supports and vehicles. The optimal percentage of the therapeutic agent in each pharmaceutical formulation varies according to the formulation itself and the therapeutic effect desired in the specific pathologies and correlated therapeutic regimens.

Inhalation and transdermal formulations can also readily be prepared.

Pharmaceutical formulation is a well-established art, and is further described in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7^{th} ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X), the disclosures of which are incorporated herein by reference in their entireties. Conventional methods, known to those of ordinary skill in the art of medicine, can be used to administer the pharmaceutical formulation(s) to the patient.

Typically, the pharmaceutical formulation will be administered to the patient by applying to the skin of the patient a transdermal patch containing the pharmaceutical formulation, and leaving the patch in contact with the patient's skin (generally for 1 to 5 hours per patch). Other transdermal routes of administration (e.g., through use of a topically applied cream, ointment, or the like) can be used by applying conventional techniques. The pharmaceutical formulation(s) can also be administered via other conventional routes (e.g., enteral, subcutaneous, intrapulmonary, transmucosal, intraperitoneal, intrauterine, sublingual, intrathecal, or intramuscular routes) by using standard methods. In addition, the pharmaceutical formulations can be administered to the patient via injectable depot routes of administration such as by using 1-, 3-, or 6-month depot injectable or biodegradable materials and methods.

Regardless of the route of administration, the therapeutic protein or antibody agent typically is administered at a daily dosage of 0.01 mg to 30 mg/kg of body weight of the patient (e.g., 1 mg/kg to 5 mg/kg). The pharmaceutical formulation can be administered in multiple doses per day, if desired, to achieve the total desired daily dose. The effectiveness of the method of treatment can be assessed by monitoring the patient for known signs or symptoms of a disorder.

The pharmaceutical compositions of the invention may be included in a container, package or dispenser alone or as part of a kit with labels and instructions for administration.

### Transgenic Animals and Cells

In another aspect, the invention provides transgenic cells and non-human organisms comprising gp354 isoform nucleic acids, and transgenic cells and non-human organisms with targeted disruption of the endogenous ortholog of the human gp354 gene. The cells can be embryonic stem cells or somatic cells. The transgenic non-human organisms can be chimeric, non-chimeric heterozygotes, and non-chimeric homozygotes.

Host cells of the invention may be used to produce non-human transgenic animals. For example, in some embodiments, a host cell of the invention is a fertilized oocyte or an embryonic stem cell into which gp354 nucleotide sequences have been introduced. Such a host cell may be used to create non-human transgenic animals in which exogenous gp354 sequences have been introduced into their genome or used to alter or replace related endogenous gp354 sequences in the animal.

As used herein, a “transgenic animal” is a non-human animal, preferably a mammal, more preferably a cow, goat, sheep, or rodent such as a rat or mouse, in which one or more of the cells of the animal includes a transgene. Other examples of transgenic animals include non-human primates, dogs, chickens, amphibians, etc.

As used herein, a “transgene” is exogenous DNA that is integrated into the genome of a cell from which a transgenic animal develops and that remains in the genome of the mature animal, thereby directing the expression of an encoded gene product in one or more cell types or tissues of the transgenic animal.

As used herein, a “homologous recombinant animal” is a non-human animal, preferably a mammal, more preferably a mouse, in which an endogenous gp354 gene has been altered by homologous recombination between the endogenous gene and an exogenous DNA molecule introduced into a cell of the animal, e.g., an embryonic cell of the animal, prior to development of the animal.

The non-human transgenic animals of the invention will be useful for studying the function and/or activity of gp354 and for identifying and/or evaluating modulators of gp354 activity. They will also be useful in methods for producing a GP354 protein or polypeptides fragment, i.e., in which the protein is produced in the mammary gland of a non-human mammal.

A transgenic animal of the invention can be created by introducing gp354-encoding nucleic acid into the male pronuclei of a fertilized oocyte, e.g., by microinjection, retroviral infection, and allowing the oocyte to develop in a pseudopregnant female foster animal. A polynucleotide comprising or having human gp354 DNA sequences of SEQ ID NO:1, 3, 5, 6, 7, 9, or 11, may be introduced as a transgene into the genome of a non-human animal. Alternatively, a non-human homolog of the human gp354 gene, such as a mouse gp354 gene, isolated by hybridization to an isolated polynucleotide of the invention, may be used as a transgene. Heterologous transcription control sequence sequences, intronic sequences, polyadenylation signals and the like may also be operatively linked with the transgene to increase the efficiency or otherwise regulate the expression (e.g., in a developmental or tissue specific manner) the transgene in the recipient host animal.

Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan 1986, In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the gp354 transgene in its genome and/or expression of gp354 mRNA in tissues or cells of the animals. A transgenic founder animal can then be used to breed additional animals carrying the transgene. Moreover, transgenic animals carrying a transgene encoding gp354 can further be bred to other transgenic animals carrying other transgenes.

To create a homologous recombinant animal, a vector is prepared which contains at least a portion of a gp354 gene into which a deletion, addition or substitution has been introduced to thereby alter, e.g., functionally disrupt, the gp354 gene. The gp354 gene can be a human gene (e.g., SEQ ID NO:1, 5, 9 or 11), but more preferably, is a non-human homolog of a human gp354 gene. For example, a mouse homolog of the human gp354 gene of SEQ ID NO:1, 5, 9 or 11 or can be used to construct a homologous recombination vector suitable for altering an endogenous gp354 gene in the mouse genome.

In some embodiments, the vector is designed such that, upon homologous recombination, the endogenous gp354 gene is functionally disrupted (i.e., no longer encodes a functional protein; also referred to as a “knock out” vector). Alternatively, the vector can be designed such that, upon homologous recombination, the endogenous gp354 gene is mutated or otherwise altered but still encodes functional protein (e.g., the upstream regulatory region can be altered to thereby alter the expression of the endogenous GP354 protein). In the homologous recombination vector, the altered portion of the gp354 gene is flanked at its 5′ and 3′ ends by additional nucleic acid of the gp354 gene to allow for homologous recombination to occur between the exogenous gp354 gene carried by the vector and an endogenous gp354 gene in an embryonic stem cell. The additional flanking gp354 nucleic acid is of sufficient length for successful homologous recombination with the endogenous gene. Typically, several kilobases of flanking DNA (both at the 5′ and 3′ ends) are included in the vector. See e.g., Thomas et al. Cell 51:503 (1987) for an exemplary description of homologous recombination vectors.

The vector is introduced into an embryonic stem cell line (e.g., by electroporation) and cells in which the introduced gp354 gene has homologously recombined with the endogenous gp354 gene are selected (see e.g., Li et al. Cell 69:915 (1992)). The selected cells are then injected into a blastocyst of an animal (e.g., a mouse) to form aggregation chimeras. See e.g., Bradley 1987, In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene.

Methods for constructing homologous recombination vectors and homologous recombinant animals are described further in Bradley, Curr. Opin. Biotechnol. 2:823-829 (1991); PCT International Publication Nos.: WO 90/11354; WO 91/01140; WO 92/0968; and WO 93/04169.

Clones of the non-human transgenic animals described herein can also be produced according to the methods described in Wilmut et al. Nature 385:810-813 (1997). In brief, a cell, e.g., a somatic cell, from the transgenic animal can be isolated and induced to exit the growth cycle and enter G0 phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

Regulated expression of transgenes in vivo may be accomplished using controllable recombination systems, such as the cre/loxP recombinase system (see, e.g., Lakso et al. Proc. Natl. Acad. Sci. USA 89:6232-6236 (1992)) and the FLP recombinase system(O'Gorman et al. Science 251:1351-1355 (1991)). If a cre/loxP recombinase system is used to regulate expression of the transgene, animals containing transgenes encoding both the Cre recombinase and a selected protein are required. Transgenic animals containing both elements of the system can be obtained, e.g., by mating two transgenic animals, each containing either the transgene encoding the selected protein or the transgene encoding a recombinase.

### Antisense Reagents and Methods

A. Antisense

Many of the isolated polynucleotides of the invention are antisense polynucleotides that recognize and hybridize to gp354 polynucleotides. Full-length and fragment antisense polynucleotides are provided. Fragment antisense molecules of the invention include (i) those that specifically recognize and hybridize to gp354 RNA (as determined by sequence comparison of DNA encoding GP354 to DNA encoding other known molecules). Identification of sequences unique to GP354 encoding polynucleotides can be deduced through use of any publicly available sequence database, and/or through use of commercially available sequence comparison programs. After identification of the desired sequences, isolation through restriction digestion or amplification using any of the various polymerase chain reaction techniques well known in the art can be performed. Antisense polynucleotides are particularly relevant to regulating expression of GP354 by those cells expressing gp354 mRNA.

Antisense oligonucleotides, or fragments of a nucleotide sequence set forth in SEQ ID NO:1, 3, 5, 6, 7, 9 or 11, or sequences complementary or homologous thereto, derived from the nucleotide sequences encoding GP354 are useful as diagnostic tools for probing gene expression in various tissues. For example, tissue can be probed in situ with oligonucleotide probes carrying detectable groups by conventional autoradiography techniques to investigate native expression of this enzyme or pathological conditions relating thereto. In specific aspects, antisense nucleic acid molecules are provided that comprise a sequence complementary to at least about 10, 25, 50, 100, 250 or 500 nucleotides or an entire gp354 coding strand, or to only a portion thereof. Nucleic acid molecules encoding fragments, homologs, derivatives and analogs of a GP354 protein of SEQ ID NO:2, 4, 8, 10 or 12, antisense nucleic acids complementary to a GP354 nucleic acid sequence of SEQ ID NO:1, 3, 5, 6, 7, 9 or 11 are additionally provided.

Antisense nucleic acid molecules of the invention may be antisense to a “coding region” or non-coding regions of the coding strand of a nucleotide sequence encoding GP354. The term “coding region” refers to the region of the nucleotide sequence comprising codons which are translated into amino acid residues (e.g., a protein coding region of human GP354 corresponds to the coding region presented in SEQ ID NO:1, 7 or 11).

Antisense oligonucleotides are preferably directed to a regulatory region of a nucleotide sequence of SEQ ID NO:1, 7 or 11, or mRNA corresponding thereto, including, but not limited to, the initiation codon, TATA box, enhancer sequences, and the like. The antisense nucleic acid molecule can be complementary to the entire coding or non-coding region of gp354, but more preferably is an oligonucleotide that is antisense to only a portion of the coding or non-coding region of gp354 mRNA. For example, the antisense oligonucleotide can be complementary to the region surrounding the translation start site of gp354 mRNA. An antisense oligonucleotide can be, for example, about 5, 10, 15, 20, 25, 30, 35, 40, 45 or 50 nucleotides in length.

Antisense nucleic acids of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention (preferably oligonucleotides of 10 to 20 nucleotides in length) are typically administered to a subject or generated in situ such that they hybridize with or bind to cellular mRNA and/or genomic DNA encoding a GP354 protein to thereby inhibit expression of the protein, e.g., by inhibiting transcription and/or translation. Suppression of gp354 expression at either the transcriptional or translational level is useful to generate cellular or animal models for diseases/conditions characterized by aberrant gp354 expression.

The hybridization can be by conventional nucleotide complementarity to form a stable duplex, or, for example, in the case of an antisense nucleic acid molecule that binds to DNA duplexes, through specific interactions in the major groove of the double helix. Phosphorothioate and methylphosphonate antisense oligonucleotides are specifically contemplated for therapeutic use by the invention. The antisense oligonucleotides may be further modified by adding poly-L-lysine, transferrin, polylysine, or cholesterol moieties at their 5′ end.

An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet other embodiments, the antisense nucleic acid molecule of the invention is an a-anomeric nucleic acid molecule. An a-anomeric nucleic acid molecule forms specific double-stranded hybrids with complementary RNA in which, contrary to the usual b-units, the strands run parallel to each other (Gaultier et al. Nucl. Acids Res 15: 6625-6641 (1987)). The antisense nucleic acid molecule can also comprise a 2′-O-methylribonucleotide (Inoue et al. Nucl. Acids Res 15: 6131-6148 (1987)) or a chimeric RNA-DNA analogue (Inoue et al. FEBS Lett 215: 327-330 (1987)).

B. Ribozymes and Catalytic Nucleic Acids

In still another series of embodiments, an antisense nucleic acid of the invention is part of a gp354 specific ribozyme (or, as modified, a “nucleozyme”). Ribozymes are catalytic RNA molecules with ribonuclease activity that are capable of cleaving a single-stranded nucleic acid, such as an mRNA, to which they have a complementary region. Thus, ribozymes (such as hammerhead, hairpin, Group I intron ribozymes, and the like) can be used to catalytically cleave gp354 mRNA transcripts to thereby inhibit translation of gp354 mRNA. A ribozyme having specificity for a gp354-encoding nucleic acid can be designed based upon the nucleotide sequence of a gp354 polynucleotide disclosed herein (SEQ ID NO:1, 3, 5, 6, 7, 9, or 11). See, e.g., U.S. Pat. Nos. 5,116,742; 5,334,711; 5,652,094; and 6,204,027, incorporated herein by reference in their entireties.

For example, a derivative of a Tetrahymena L-19 IVS RNA can be constructed in which the nucleotide sequence of the active site is complementary to the nucleotide sequence to be cleaved in a GP354-encoding mRNA. See, e.g., Cech et al. U.S. Pat. No. 4,987,071; and Cech et al. U.S. Pat. No. 5,116,742. Alternatively, gp354 mRNA can be used to select a catalytic RNA having a specific ribonuclease activity from a pool of RNA molecules. See, e.g., Bartel et al., Science 261:1411-1418 (1993).

Expression of the gp354 gene may be inhibited by targeting nucleotide sequences complementary to the regulatory region of the gp354 (e.g., the gp354 promoter and/or enhancers) to form triple helical structures that prevent transcription of the gp354 gene in target cells. See generally, Helene, Anticancer Drug Des. 6: 569-84 (1991); Helene et al. Ann. N.Y. Acad. Sci. 660:27-36 (1992); and Maher Bioassays 14: 807-15 (1992).

C. Peptide Nucleic Acids (PNA)

In other preferred oligonucleotide mimetics, especially useful for in vivo administration, both the sugar and the internucleoside linkage are replaced with novel groups, such as peptide nucleic acids (PNA). See, e.g., Hyrup et al. Bioorg. Med. Chem. Lett. 4:5-23 (1996). In PNA compounds, the phosphodiester backbone of the nucleic acid is replaced with an amide-containing backbone, in particular by repeating N-(2-aminoethyl) glycine units linked by amide bonds. Nucleobases are bound directly or indirectly to aza-nitrogen atoms of the amide portion of the backbone, typically by methylene carbonyl linkages. The synthesis of PNA oligomers can be performed using standard solid phase peptide synthesis protocols as described in Hyrup et al., supra; and Perry-O'Keefe et al., Proc. Natl. Acad. Sci. USA 93:14670-675 (1996).

PNAs of gp354 can be used in therapeutic and diagnostic applications. For example, PNAs can be used as antisense or antigene agents for sequence-specific modulation of gene expression by, e.g., inducing transcription or translation arrest or inhibiting replication. PNAs of gp354 can also be used, e.g., in the analysis of single base pair mutations in a gene by, e.g., PNA directed PCR clamping; as artificial restriction enzymes when used in combination with other enzymes, e.g., S1 nucleases; or as probes or primers for DNA sequence and hybridization (Hyrup et al., supra; and Perry-O'Keefe, supra).

In other embodiments, PNAs of gp354 can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of gp354 can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup et al., supra). The synthesis of PNA-DNA chimeras can be performed as described in Hyrup, supra and Finn et al., Nucl. Acids Res. 24:3357-63 (1996).

For example, a DNA chain can be synthesized on a solid support using standard phosphoramidite coupling chemistry, and modified nucleoside analogs, e.g., 5′-(4-methoxytrityl) amino-5′-deoxy-thymidine phosphoramidite, can be used between the PNA and the 5′ end of DNA (Mag et al., Nucl. Acids Res. 17:5973-88 (1989)). PNA monomers are then coupled in a stepwise manner to produce a chimeric molecule with a 5′ PNA segment and a 3′ DNA segment (Finn et al., supra). Alternatively, chimeric molecules can be synthesized with a 5′ DNA segment and a 3′ PNA segment. See, Petersen et al., Bioorg. Med. Chem. Lett. 5:1119-11124 (1975).

In other embodiments, the oligonucleotide may include other appended groups such as peptides (e.g., for targeting host cell receptors in vivo), or agents facilitating transport across the cell membrane (see, e.g., Letsinger et al., Proc. Natl. Acad. Sci. USA 86:6553-6556 (1989); Lemaitre et al., Proc. Natl. Acad. Sci. USA 84:648-652 (1987); PCT Publication No. W088/09810) or the blood-brain barrier (see, e.g., PCT Publication No. W089/10134). In addition, oligonucleotides can be modified with hybridization triggered cleavage agents (See, e.g., Krol et al., BioTechniques 6:958-976 (1988)), or intercalating agents (See, e.g., Zon, Pharm. Res. 5: 539-549 (1988)). To this end, the oligonucleotide may be conjugated to another molecule, e.g., a peptide, a hybridization triggered cross-linking agent, a transport agent, a hybridization-triggered cleavage agent, etc.

PNA chemistry and applications are reviewed, inter alia, in Ray et al., FASEB J. 14(9):1041-60 (2000); Nielsen et al., Pharmacol Toxicol. 86(1):3-7 (2000); Larsen et al., Biochim Biophys Acta. 1489(1):159-66 (1999); Nielsen, Curr. Opin. Struct. Biol. 9(3):353-7 (1999), and Nielsen, Curr. Opin. Biotechnol. 10(1):71-5 (1999), the disclosures of which are incorporated herein by reference in their entireties.

### Diagnostic Methods

A. Nucleic Acid Diagnostics

As described above, the isolated polynucleotides of the invention can be used as nucleic acid probes to assess the levels of gp354 mRNA in tissues in which it is normally expressed (e.g., pancreas and CNS), and in tissues in which it is not normally expressed, if such abnormal tissue mis-expression is suspected.

The invention thus provides a method for detecting the presence of a gp354 polynucleotide in a biological sample (e.g., a cell extract, fluid or tissue sample derived from a patient) by contacting the sample with an isolated polynucleotide of the invention which is capable of specifically detecting by hybridization gp354 polynucleotide sequences.

Preferably, the method comprises the steps of contacting the sample with an the isolated nucleic acid under high stringency hybridization conditions and detecting hybridization of the isolated polynucleotide to a nucleic acid in the sample, wherein the occurrence of said hybridization indicates the presence of a gp354-encoding sequence in the sample.

The isolated polynucleotides of the invention can be used as nucleic acid probes that are specific to particular cell types in the pancreas and central nervous system based on the specific expression of gp354 in these tissued. Accordingly, the present invention provides a method for identifying a cell as a pancreatic or a neural cell by detecting the presence of a gp354 polynucleotide in a biological sample (e.g., a cell extract, fluid or tissue sample derived from a patient) by contacting the sample with an isolated polynucleotide of the invention which is capable of specifically detecting by hybridization gp354 polynucleotide sequences.

The present invention also provides a diagnostic assay for identifying the presence or absence of a genetic lesion or mutation characterized by at least one of: (i) aberrant modification or mutation of a gene encoding a GP354 protein; (ii) mis-regulation of a gene encoding a GP354 protein; and (iii) aberrant post-translational modification of a GP354 protein, wherein a wild-type form of the gene encodes a protein with a GP354 biological activity.

The present invention further provides a method of identifying a homolog of a human gp354 gene, comprising the step of hybridizing a nucleic acid library with a nucleic acid probe comprising SEQ ID NO:1, 3, 5, 6, 7, 9 or 11, or a portion thereof having at least 17 nucleotides, under medium or high stringency hybridization conditions; and determining whether the nucleic acid probe hybridizes to a nucleic acid sequence in the library. If the nucleic acid sequence in the library hybridizes under such selected conditions, it is a homolog of a human gp354 gene.

B. Antibody Diagnostics

Antibodies of the present invention can be used to assess the expression levels of GP354 proteins in tissues in which it is normally expressed (e.g., pancreas and CNS), and in tissues in which it is not normally expressed, if such abnormal tissue mis-expression is suspected.

The invention thus provides a method for detecting the presence of a GP354 protein or its activity in a biological sample (e.g., a cell extract, fluid or tissue sample derived from a patient) by contacting the sample with an agent capable of detecting an indicator of the presence of GP354 protein or its activity. Preferably, the agent is an antibody specific for at least one epitope of GP354 protein.

Accordingly, the invention provides a method for determining whether a GP354 protein is present in a sample, comprising the step of contacting the sample with an antibody having at least one GP354 epitope and detecting specific binding of the antibody to an antigen, which indicates the presence of a GP354 protein in the sample.

The above method will also be useful for identifying a test cell in a subject as a pancreatic or a neural cell by comparing the amount of GP354 polypeptides present in a biological sample (e.g., a cell extract, fluid or tissue sample derived from the subject) from the subject test cell to the amount of GP354 polypeptides present in a parallel biological sample from non-pancreatic or non-neural tissue.

In some embodiments, anti-PanCAM antibodies described herein can be used to quantify pancreatic islet injury in early type I diabetes, as well as any therapy aimed at restoring islet function. For instance, the antibodies are attached to a contrast marker (e.g., Gadolinium) for MRI and CT applications, or to a radioactive marker (e.g., Technecium) for nuclear medicine applications. The relevant diagnostic modality is then used to quantify and document changes in islet sizes and/or numbers. The antibodies so labeled may also localize islet cell tumors using such a diagnostic modality.

C. Methods for Diagnosing Disease

The gp354 isolated polynucleotides, proteins and GP354 specific antibodies of the invention will be useful in methods for diagnosing a variety of disorders and disease conditions associated with aberrant gp354 expression.

The invention thus provides a method for diagnosing a disease condition in a subject, comprising the steps of comparing the amount or activity of a GP354 protein in a tissue sample from the subject to the amount or activity of the GP354 polypeptide in a control sample (e.g., an equivalent one derived from a healthy subject), wherein a significant difference in the amount or activity of the GP354 polypeptide in the tissue sample relative to the amount or activity of the GP354 polypeptide in the control sample indicates that the subject has a disease condition.

In preferred embodiments, the amount or activity of a GP354 protein in a tissue sample is assessed by competitive binding assays using a GP354 polypeptides or fragment of the invention, or by an immunoassay using a GP354 specific antibody of the invention. Preferably, the method is used to diagnose a disease condition relating to the pancreas or to the nervous system.

Also provided are methods for diagnosing a disease condition in a subject by monitoring relative gp354 mRNA levels in difference tissues. Preferably, the methods comprise the step of comparing the amount of a gp354 mRNA in a test tissue sample from the subject to the amount of gp354 mRNA in a control sample, wherein a significant difference in the amount of the mRNA in the test sample relative to the amount in the control sample indicates that the subject has a disease condition.

In preferred embodiments, the amount of gp354 mRNA in a tissue sample is assessed by hybridization using an isolated gp354 polynucleotide or nucleic acid fragment of the invention. Preferably, the method is used to diagnose a disease condition relating to the pancreas or to the nervous system.

### Computer Readable Means

A further aspect of the invention is a computer readable means for storing the gp354 nucleic acid and amino acid sequences of the instant invention. In preferred embodiments, the invention provides a computer readable means for storing SEQ ID NOs as described herein, as the complete set of sequences or in any combination. The records of the computer readable means can be accessed for reading and display and for interface with a computer system for the application of programs allowing for the location of data upon a query for data meeting certain criteria, the comparison of sequences, the alignment or ordering of sequences meeting a set of criteria, and the like.

The nucleic acid and amino acid sequences of the invention are particularly useful as components in databases useful for search analyses as well as in sequence analysis algorithms. As used in these embodiments, the terms “nucleic acid sequences of the invention” and “amino acid sequences of the invention” mean any detectable chemical or physical characteristic of a polynucleotide or polypeptide of the invention that is or may be reduced to or stored in a computer readable form. These include, without limitation, chromatographic scan data or peak data, photographic data or scan data therefrom, and mass spectrographic data.

This invention provides computer readable media having stored thereon sequences of the invention. A computer readable medium may comprise one or more of the following: a nucleic acid sequence comprising a sequence of a nucleic acid sequence of the invention; an amino acid sequence comprising an amino acid sequence of the invention; a set of nucleic acid sequences wherein at least one of said sequences comprises the sequence of a nucleic acid sequence of the invention; a set of amino acid sequences wherein at least one of said sequences comprises the sequence of an amino acid sequence of the invention; a data set representing a nucleic acid sequence comprising the sequence of one or more nucleic acid sequences of the invention; a data set representing a nucleic acid sequence encoding an amino acid sequence comprising the sequence of an amino acid sequence of the invention; a set of nucleic acid sequences wherein at least one of said sequences comprises the sequence of a nucleic acid sequence of the invention; a set of amino acid sequences wherein at least one of said sequences comprises the sequence of an amino acid sequence of the invention; a data set representing a nucleic acid sequence comprising the sequence of a nucleic acid sequence of the invention; a data set representing a nucleic acid sequence encoding an amino acid sequence comprising the sequence of an amino acid sequence of the invention. The computer readable medium can be any composition of matter used to store information or data, including, for example, commercially available floppy disks, tapes, hard drives, compact disks, and video disks.

Accordingly, the invention provides a diagnostic assay for identifying a homolog of a human gp354 gene, comprising the step of screening a nucleic acid database with a query sequence consisting of SEQ ID NO:1, 3, 5, 6, 7, 9 or 11, or a portion thereof having 300 or more nucleotides, wherein a nucleic acid sequence in said database that is at least 65% but less than 100% identical to SEQ ID NO:1, 3, 5, 6, 7, 9 or 11, or said portion thereof, if found, is a homolog of a human gp354 gene.

Also provided by the invention are methods for the analysis of character sequences, particularly genetic sequences of the invention. Preferred methods of sequence analysis include, for example, methods of sequence homology analysis, such as identity and similarity analysis, RNA structure analysis, sequence assembly, cladistic analysis, sequence motif analysis, open reading frame determination, nucleic acid base calling, and sequencing chromatogram peak analysis.

A computer-based method is provided for performing nucleic acid homology identification. This method comprises the steps of providing a nucleic acid sequence comprising the sequence of a nucleic acid of the invention in a computer readable medium; and comparing said nucleic acid sequence to at least one nucleic acid or amino acid sequence to identify homology.

A computer-based method is also provided for performing amino acid homology identification, said method comprising the steps of: providing an amino acid sequence comprising the sequence of a polypeptide of the invention in a computer readable medium; and comparing said amino acid sequence to at least one nucleic acid or an amino acid sequence to identify homology.

A computer based method is still further provided for assembly of overlapping nucleic acid sequences into a single nucleic acid sequence, said method comprising the steps of: providing a first nucleic acid sequence comprising the sequence of a nucleic acid of the invention in a computer readable medium; and screening for at least one overlapping region between said first nucleic acid sequence and a second nucleic acid sequence.

### EXAMPLES

The following example is meant to illustrate the methods and materials of the present invention. Suitable modifications and adaptations of the described conditions and parameters normally encountered in the art of molecular biology which are apparent to those skilled in the art are within the spirit and scope of the present invention.

For the experiments described below, all RT-PCR and fragments were gel-purified prior to cloning. The fragments were separated by agarose gel electrophoresis by standard methods. DNA fragments were excised from the agarose gel and purified from the gel using QIAEX resin according to the manufacturer's specifications (Qiagen, Valencia, Calif.). The gel-purified fragments were cloned into plasmid vectors and then the plasmids were used to transform competent TOP10 E. coli host cells. Plasmids produced by the host cells were isolated by a standard alkaline lysis miniprep procedure (Qiagen, Valencia, Calif.). Sequencing was executed by a standard dideoxy termination method (Applied Biosystems, Foster City, Calif.).

### Example 1

### In Silico Gene Prediction and Sequence Analysis

The gene prediction software programs GENSCAN (Burge et al., J. Mol. Biol. 268:78-94 (1997)) and GENEMARKHMM (Lukashin et al., Nucl. Acids Res. 26:1107-1115 (1998)) were used to identify novel genes in the high throughput genomic sequences deposited in GenBank. To do so, the Genbank data entries were downloaded to a local server, and individual sequence contigs were separated according to the annotation provided with the sequence entries. The parameters used in the analyses were the default parameters included with the programs (Burge et al., supra; and Lukashin et al., supra).

Gene candidates with at least two identical exons predicted from GENSCAN and GENEMARKHMM were further analyzed. Specifically, the gene sequences were translated to protein sequences which were in turn used as queries in Blast analyses of a non-redundant protein sequence database incorporating Genpept, Swissprot, and PIR. The protein sequences were also analyzed for signal peptides using SIGNALP (Nielsen et al., Int J. Neural Syst 8:581-99 (1997)). Candidate genes encoding proteins with signal peptides and similarity to Ig superfamily proteins were subjected to experimental verification and cloning.

The BLAST (“Basic Local Alignment Search Tool”) algorithm is suitable for determining sequence similarity (Altschul et al., J. Mol. Biol., 215:403-410 (1990)). Software for performing BLAST analyses is publicly available through the National Center for Biotechnology Information website. This algorithm involves first identifying high scoring sequence pair (HSPs) by identifying short words of length W in the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: (1) the cumulative alignment score falls off by the quantity X from its maximum achieved value; (2) the cumulative score goes to zero or below, due to the accumulation of one or more negative-scoring residue alignments; or (3) the end of either sequence is reached. The BLAST algorithm parameters W, T and X determine the sensitivity and speed of the alignment. The BLAST program uses as defaults a word length (W) of 11, the BLOSUM62 scoring matrix (see Henikoff et al., Proc. Natl. Acad. Sci. USA, 89:10915-10919 (1992)) alignments (B) of 50, expectation (E) of 10, M=5, N=4, and a comparison of both strands.

BLAST (Karlin et al., Proc. Natl. Acad. Sci. USA, 90:5873-5787 (1993)) and GAPPED BLAST perform a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a nucleic acid is considered similar to a gp354 gene or cDNA if the smallest sum probability in comparison of the test nucleic acid to gp354 is less than about 1, preferably less than about 0.1, more preferably less than about 0.01, and most preferably less than about 0.001.

The gp354 gene (ORF) was identified in contig 38 of a BAC with the GenBank accession number AC022315, which was deposited on Feb. 10, 2000. The GENSCAN prediction for this gene was in the reverse orientation and included the following 14 exons, shown in TABLE 3.

TABLE 3
GENSCAN results

| Exon | Begin | End | Length |
|---|---|---|---|
| 14 | 1844 | 1779 | 66 |
| 13 | 3567 | 3464 | 104 |
| 12 | 4007 | 3903 | 105 |
| 11 | 4695 | 4476 | 220 |
| 10 | 4959 | 4859 | 101 |
| 09 | 5378 | 5246 | 133 |
| 08 | 5591 | 5464 | 128 |
| 07 | 5981 | 5833 | 149 |
| 06 | 6203 | 6098 | 106 |
| 05 | 7019 | 6869 | 151 |
| 04 | 7796 | 7636 | 161 |
| 03 | 8092 | 7943 | 150 |
| 02 | 9157 | 9008 | 150 |
| 01 | 9373 | 9322 | 52 |

BLAST analysis of the gp354 gene against publicly available EST databases showed no ESTs that matched the predicted gene.

### Example 2

### Amplification of gp354

A sequence of gp354 cDNA is obtained by performing rapid amplification of cDNA ends (RACE) using the MARATHON-READY RACE kit (Clontech, Palo Alto, Calif.) A MARATHON-READY cDNA is a double-stranded sDNA synthesized from human tissue mRNA and ligated to a standard set of adapters (Clontech). All RACE reactions use an adapter primer AP-1, 5′-CCATCCTAATACGACTCACTATAGGGC-3′ (SEQ ID NO:15) provided with the kit. The 3′ RACE for gp354 may use AP-1 together with the forward primer GX1-218, 5′-ACTGGGGGCTAGTTCAGTGGACTAA-3′ (SEQ ID NO:16), or the complement of the reverse primer, GX1-219, 5′-CCAAACAGCACATCCAGCGCAGTAC-3′ (SEQ ID NO:17). The 5′ RACE for gp354 may use AP-1 together with the reverse primer GX1-219, or the complement of the forward primer GX1-218. ADVANTAGE 2 DNA polymerase (Clontech) may be used for the amplification reactions. The MARATHON-READY kit may be used according to the manufacturer's specifications except that “touchdown” PCR (Don et al., Nuc. Acids Res. 19:4008 (1991)) conditions are used for thermal cycling. The thermal cycling conditions are as follows: 94° C. for 1 minute, one cycle of 94° C. for 15 seconds, 72° C. for 15 seconds, 68° C. for 15 seconds; one cycle of 94° C. for 15 seconds, 71° C. for 15 seconds, 68° C. for 15 seconds; one cycle of 94° C. for 15 seconds, 70° C. for 15 seconds, 68° C. for 15 seconds; one cycle of 94° C. for 15 seconds, 69° C. for 15 seconds, 68° C. for 15 seconds; 35 cycles of 94° C. for 15 seconds and 68° C. for 30 seconds; and 68° C. for 10 minutes.

### Example 3

### Confirmation of GP354 Expression by RT-PCR

Inter-exon PCR was used to confirm that the predicted gp354 gene was indeed expressed and to initiate the cloning process that would determine the true (rather than the predicted) gene structure. Templates for the PCR were single-stranded cDNAs contained in a multi-tissue cDNA panel generated by reverse transcription PCR (“RT-PCR”) from mRNA isolated from multiple human tissues according to the manufacturer's specifications (Clontech). These tissues were brain, heart, kidney, liver, lung, pancreas, pituitary, skeletal muscle, colon, ovary, peripheral blood leukocyte, prostate, small intestine, spleen, testis, and thymus.

Primers for the inter-exon PCR were selected from separate exons so that every PCR experiment had a built-in control to distinguish spliced messages from unspliced messages or from genomic contamination in the cDNA panels. One pair of such primers were GX1-220 and GX1-221 (Example 13, inpra). Another pair used were GX1-218 and GX1-219 (supra). The PCR was carried out according to the specification included with the multiple tissue cDNA panel (Clontech). Typical thermal cycler conditions for the PCR were: 94° C. for 1 minute, followed by 35 cycles of 94° C. for 20 seconds, 68° C. for 2 minutes, followed by 5 minutes at 68° C. after the last cycle. PCR products were separated on 1.0% agarose gel and visualized by ethidium bromide staining.

The results showed a fragment of approximately 785 bp in the pancreas and in no other tissues. The same observation was made when GX1-218 and GX1-219. This fragment was isolated and cloned into the PCR2.1 plasmid vector (Invitrogen, Carlsbad, Calif.). The resultant plasmid construct CS0026 was propagated in TOP10 E. coli host cells. Plasmids were purified by a standard alkaline lysis miniprep procedure (Qiagen, Valencia, Calif.). Sequencing was executed by standard di-deoxy terminator methods (Applied Biosystems, Foster City, Calif.). The sequence of the PCR fragment (SEQ ID NO:3) was compared with the sequences of the predicted gp354 gene to confirm the accuracy of the gene prediction.

### Example 4

### Identification of Full-Length gp354 cDNA by RACE

Because the gene prediction programs GENSCAN and GENEMARK have predictable error rates (Burge et al., supra; Lukashin et al., supra), the PCR fragment described in Example 3 are used as a seed sequence to obtain the rest of the gp354 cDNA sequence via RACE reactions. For the 3′ RACE reaction, the primer is GX1-218 or the complement of GX1-219, and the template is cDNAs derived from human pancreas tissue (see Example 3). For the 5′ RACE, the primer is GX1-219 or the complement of GX1-218, and the template is also cDNAs derived from human pancreas tissue. The 5′ and 3′ RACE fragments so obtained are gel-purified, cloned, and sequenced. To assemble the full-length gp354 cDNA sequence, the initial PCR product, the 5′ RACE product and the 3′ RACE product are assembled into a single contiguous sequence using the ASSEMBLE program in the GCG computer package (Genetics Computer Group, Madison, Wis.).

### Example 5

### Confirmation of GP354 Expression by Northern Blot Analysis

To confirm the expression of GP354, Northern blot analysis was conducted with each lane of the blot (Clontech catalogue no. 7760-1) containing 2 μg of polyA RNA. The tissues represented on the blot included heart, brain, placenta, lung, liver, skeletal muscle, kidney, and pancreas. The probe for the Northern blot was the PCR fragment described in Example 3 (SEQ ID NO:3). 50 ng of the probe was labeled by the random-primed method of Feinberg and Vogelstein (Anal. Biochem. 132:6-13 (1983)). Hybridization was carried out at 68° C. for one hour in EXPRESSHYB solution (Clontech catalogue no. 8015-1). Prior to autoradiography, the Northern blot was washed with 2×SSC/0.05% SDS at room temperature, followed by two washes with 0.1×SSC/0.1% SDS at 50° C.

A hybridizing RNA of approximately 3.2 kb was observed in the pancreas but not in any other tissues tested.

### Example 6

### PCR Screening of A Genomic Library and Subcloning of GP354 Coding Regions

Subcloning of the gp354 genomic locus may be accomplished by PCR from a genomic library, or directly from genomic DNA. For example, two microliters of a human genomic library (˜10⁸ PFU/ml) (Clontech) are added to 6 ml of an overnight culture of K802 cells (Clontech), and then distributed as 250 ml aliquots into each of 24 microtubes. The microtubes are incubated at 37° C. for 15 min. Seven milliliters of 0.8% agarose is added to each tube, mixed, then poured onto LB agar +10 mM MgSO₄ plates and incubated overnight at 37° C. To each plate 5 ml of SM phage buffer (0.1 M NaCl, 8.1 mM MgSO₄.7H₂O, 50 mM Tris.Cl (pH 7.5), 0.01% gelatin) is added and the top agarose is removed with a microscope slide and placed in a 50 ml centrifuge tube. A drop of chloroform is added and the tube is placed in a 37° C. shaker for 15 min, then centrifuged for 20 min at 4000 rpm (Sorvall RT6000 table top centrifuge) and the supernatant stored at 4° C. as a stock solution.

PCR may be then performed in 20 ml containing 8.8 ml H₂O, 4 ml 5×RAPID-LOAD BUFFER (Origene), 2 ml 10×PCR BUFFER II (Perkin Elmer), 2 ml 25 mM MgCl2, 0.8 ml 10 mM dNTP, 0.12 ml of a primer comprising at least a portion of the sequence of the 5′ end of the gp354 polynucleotide of SEQ ID NO:1 (1 mg/ml), 0.12 ml of a primer comprising at least a portion of the sequence that is complementary to the 3′ end of the gp354 polynucleotide of SEQ ID NO:1 (1 mg/ml), 0.2 ml AMPLITAQ GOLD polymerase (Perkin Elmer) and 2 ml of phage solution from each of the 24 tubes. The PCR reaction involves 1 cycle at 80° C. for 20 min, 95° C. for 10 min, then 22 cycles at 95° C. for 30 sec, 72° C. for 4 min decreasing 1° C. each cycle, 68° C. for 2 min, followed by 30 cycles at 95° C. for 30 sec, 55° C. for 30 sec, 68° C. for 60 sec. The reaction is loaded onto a 2% agarose gel.

From the tube that gives a PCR product of the correct size, 5 μl is used to set up five 1:10 dilutions that are plated onto LB agar +10 mM MgSO₄ plates and incubated overnight. A BA85 nitrocellulose filter (Schleicher & Schuell) is placed on top of each plate for 1 hour. The filter is removed, placed with the phage side up in a petri dish, and covered with 4 ml of SM buffer for 15 min to elute the phage. One milliliter of SM buffer is removed from each plate and used to set up a PCR reaction as described above. The plate of the lowest dilution to give a PCR product is subdivided, filter-lifted and the PCR reaction is repeated. The series of dilutions and subdividions of the plate is continued until a single plaque is isolated that gives a positive PCR band. Once a single plaque is isolated, 10 ml phage supernatant is added to 100 ml SM and 200 ml of K802 cells per plate with a total of 8 plates set up. The plates are incubated overnight at 37° C. Eight milliliters of SM is added to each plate, and the top agarose is scraped off with a microscope slide and collected in a centrifuge tube.

Three drops of chloroform are added to the centrifuge tube. Subsequently, the tube is vortexed, incubated at 37° C. for 15 min, and centrifuged for 20 min at 4000 rpm (Sorvall RT6000 table top centrifuge) to recover the phage. The recovered phage is used to isolate genomic phage DNA using the QIAGEN LAMBDA MIDI KIT. The sequences for primers may be derived from the sequences given herein.

To subclone the coding region of the gp354 gene, PCR is performed in a 50 μl reaction containing 33 μl H₂O, 5 μl 10×TT buffer (140 mM ammonium sulfate, 0.1% gelatin, 0.6 M Tris-tricine pH 8.4), 5 μl 15 mM MgSO₄, 2 μl 10 mM dNTP, 4 μl genomic phage DNA (0.1 μg/ml), 0.3 μl of a primer comprising at least a portion of the 5′ most coding sequence of the gp354 polynucleotide of SEQ ID NO:1 (1 μg/ml), 0.3 μl of a primer comprising a sequence that is complementary to at least a portion of the 3′ most coding sequence of the gp354 polynucleotide of SEQ ID NO:1 (1 μg/ml), 0.4 μl HIGH FIDELITY Taq polymerase (Boehringer Mannheim). The PCR reaction is started with 1 cycle of 94° C. for 2 min followed by 15 cycles at 94° C. for 30 sec, 55° C. for 60 sec., and 68° C. for 2 min.

The PCR product is loaded onto a 2% agarose gel. The DNA band of expected size is excised from the gel, placed in GENELUTE AGAROSE spin column (Supelco) and spun for 10 min at maximum speed. The eluted DNA is ethanol-precipitated and resuspended in 12 μl H₂O for ligation. The PCR primer sequences may be derived from the sequences provided herein.

The ligation reaction uses solutions from the TOPO TA Cloning Kit (Invitrogen). The reaction proceeds in a solution containing 4 μl of PCR product and 1 μl of pCRII-TOPO vector at room temperature for 5 min. The reaction is terminated by the addition of 1 μl of 6×TOPO Cloning Stop Solution. The ligation product is then placed on ice. Two microliters of the ligation reaction is used to transform ONE-SHOT TOP10 cells (Invitrogen). Briefly, the ligation reaction is mixed with the cells and placed on ice for 30 min. The cells are then heat-shocked for 30 seconds at 42° C. and placed on ice for two minutes. Next, 250 μl of SOC is added to the cells, which are incubated at 37° C. with shaking for one hour and then plated onto ampicillin plates.

A single colony from the plates is used to inoculate a 5 ml culture of LB medium. Plasmid DNA is purified from the culture using the CONCERT RAPID PLASMID MINIPREP SYSTEM (GibcoBRL) and the insert of the plasmid DNA is then sequenced.

The gp354 genomic phage DNA may be sequenced using the ABI PRISM 310 Genetic Analyzer (PE Applied Biosystems), which uses the advanced capillary electrophoresis technology and the ABI PRISM BIGDYE Terminator Cycle Sequencing Ready Reaction Kit. The cycle-sequencing reaction may contain 14 ml of H₂O, 16 ml of BIGDYE Terminator mix, 7 ml genomic phage DNA (0.1 mg/ml), and 3 ml primer (25 ng/ml). The reaction is performed in a Perkin-Elmer 9600 thermocycler at 95° C. for 5 min, followed by 99 cycles of 95° C. for 30 sec, 55° C. for 20 sec, and 60° C. for 4 min. The product is purified using a CENTRIFLEX gel filtration cartridge, dried under vacuum, and then dissolved in 16 μl of Template Suppression Reagent (PE Applied Biosystems). The samples are heated at 95° C. for 5 min and then placed in the 310 Genetic Analyzer.

The DNA subcloned into pCRII is sequenced using the ABI PRISM 310 Genetic Analyzer, supra. Each cycle-sequencing reaction contains 6 ml of H₂0, 8 ml of BIGDYE Terminator mix, 5 ml of miniprep DNA (0.1 mg/ml), and 1 ml of primer (25 ng/ml) and is performed in a Perkin-Elmer 9600 thermocycler with 25 cycles of 96° C. for 10 sec, 50° C. for 10 sec, and 60° C. for 4 min. The product is purified using a CENTRIFLEX gel filtration cartridge, dried under vacuum, and then dissolved in 16 μl of Template Suppression Reagent. The samples are heated at 95° C. for 5 min and then placed in the 310 Genetic Analyzer.

### Example 7

### Hybridization Analysis To Demonstrate GP354 Expression in Brain

The expression of gp354 in mammals, such as rat, may be investigated by in situ hybridization histochemistry. To investigate gp354 expression in the pancreas, for example, coronal and sagittal rat pancreas cryosections (20 μm thick) are prepared using a Reichert-Jung cryostat. Individual sections are thaw-mounted onto silanized, nuclease-free slides (CEL Associates, Inc., Houston, Tex.), and stored at −80° C. Sections are processed starting with post-fixation in cold 4% paraformaldehyde, rinsed in cold phosphate-buffered saline (PBS), acetylated using acetic anhydride in triethanolamine buffer, and dehydrated through a series of alcohol washes in 70%, 95%, and 100% alcohol at room temperature. Subsequently, sections are delipidated in chloroform, followed by rehydration through successive exposure to 100% and 95% alcohol at room temperature. Microscope slides containing processed cryosections are allowed to air dry prior to hybridization. Other tissues may be assayed in a similar fashion.

A gp354-specific probe may be generated using PCR and sequence information from SEQ ID NO:1 or SEQ ID NO:3. Following PCR amplification, the fragment is digested with restriction enzymes and cloned into pBluescript II cleaved with the same enzymes. For production of a probe specific for the sense strand of gp354, a cloned gp354 fragment cloned in pBluescript II may be linearized with a suitable restriction enzyme, which provides a substrate for labeled run-off transcripts (i.e., cRNA riboprobes) using the vector-borne T7 promoter and commercially available T7 RNA polymerase. A probe specific for the antisense strand of gp354 may also be readily prepared using the gp354 clone in pBluescript II by cleaving the recombinant plasmid with a suitable restriction enzyme to generate a linearized substrate for the production of labeled run-off cRNA transcripts using the T3 promoter and cognate polymerase.

The riboprobes may be labeled with [³⁵S]-UTP to yield a specific activity of about 0.40×10⁶ cpm/pmol for antisense riboprobes and about 0.65×10⁶ cpm/pmol for sense-strand riboprobes. Each riboprobe may be subsequently denatured and added (2 pmol/ml) to hybridization buffer which contains 50% formamide, 10% dextran, 0.3 M NaCl, 10 mM Tris (pH 8.0), 1 mM EDTA, 1×Denhardt's Solution, and 10 mM dithiothreitol.

Microscope slides containing sequential pancreas cryosections may be independently exposed to 45 μl of hybridization solution per slide and silanized cover slips may be placed over the sections being exposed to hybridization solution. Sections are incubated overnight (e.g., 15-18 hours) at 52° C. to allow hybridization to occur. Equivalent series of cryosections are then exposed to sense or antisense gp354-specific cRNA riboprobes.

Following the hybridization period, coverslips are washed off the slides in 1×SSC, followed by RNase A treatment by exposing the slides to 20 μg/ml RNase A in a buffer containing 10 mM Tris.HCl (pH 7.4), 0.5 M EDTA, and 0.5 M NaCl for 45 minutes at 37° C. The cryosections are then subjected to three high-stringency washes in 0.1 ×SSC at 52° C. for 20 minutes each. Following the series of washes, cryosections are dehydrated by consecutive exposure to 70%, 95%, and 100% ammonium acetate in alcohol, followed by air drying and exposure to KODAK BIOMAX MR-1 film. After 13 days of exposure, the film is developed, and any significant hybridization signal is detected.

Based on these results, slides containing tissue that hybridized, as shown by film autoradiograms, are coated with KODAK NTB-2 nuclear track emulsion and the slides are stored in the dark for 32 days. The slides are then developed and counterstained with hematoxylin. Emulsion-coated sections are analyzed microscopically to determine the specificity of labeling. The signal is determined to be specific if autoradiographic grains (generated by antisense probe hybridization) are clearly associated with cresyl violate-stained cell bodies. Autoradio-graphic grains found between cell bodies indicate non-specific binding of the probe.

Expression of GP354 in the pancreas and the brain (infra) provides an indication that modulators of GP354 activity have utility for treating certain neural disorders by inhibiting or increasing the activity of GP354 in the nervous system.

### Example 8

### Northern Blot Analysis of gp354-RNA

Northern blot hybridizations may be performed to examine the expression of gp354 mRNA. A clone containing at least a portion of the sequence of SEQ ID NO:1, SEQ ID NO:3, or a complement thereto, may be used as a probe. Vector-specific primers are used in PCR to generate a hybridization probe fragment for ³²P-labeling. The PCR is performed as follows: (1) mix the following reagents:

| | |
|---|---|
| 1 μl | gp354-containing plasmid |
| 2 μl | forward primer |
| 2 μl | reverse primer |
| 10 μl | 10× PCR buffer provided by the manufacturer of the Taq |
| | polymerase (e.g., Amersham Pharmacia Biotech) |
| 1 μl | 10 mM dNTP (e.g., Boehringer Mannheim catalogue no. 1 |
| | 969 064) |
| 0.5 μl | Taq polymerase (such as Amersham Pharmacia Biotech |
| | catalogue no. 27-0799-62) |
| 83.5 μl | water |

(2) perform PCR in a thermocylcer using the following program: 94° C. 5 min; 30 cycles of 94° C., 1 min, 55° C., 1 min, and 72° C. 1 min; and then 72° C., 10 min.

The PCR product may be purified using QIAQUICK PCR Purification Kit (Qiagen catalogue no. 28104). The purified PCR fragment is labeled with ³²P-dCTP (Amersham Pharmacia Biotech catalogue no. AA0005/250) by random priming using “Ready-to-go DNA Labeling Beads” (Amersham Pharmacia Biotech cat. no. 27-9240-01). Hybridization is carried out on a human multi-tissue Northern blot from Clontech according to the manufacturer's protocol. After overnight exposure on a Molecular Dynamics PHOSPHORIMAGER screen (cat. no. MD146-814), bands of about 1.35 kb are visualized.

### Example 9

### Recombinant Expression of GP354 in Eukaryotic Host Cells

A. Expression of gp354 in Mammalian Cells

To produce GP354 protein, a GP354-encoding polynucleotide is expressed using recombinant techniques. For example, the GP354-encoding sequence described in Example 1 is subcloned into the commercial expression vector pzeoSV2 (Invitrogen). The resultant expression construct is transfected into Chinese Hamster Ovary (CHO) cells using the transfection reagent FUGENE6 (Boehringer-Mannheim) and the transfection protocol provided in the product insert. Other eukaryotic cell lines, including human embryonic kidney (HEK 293) and COS cells, are suitable as well.

Cells stably expressing GP354 are selected by growth in the presence of 100 μg/ml zeocin (Stratagene, LaJolla, Calif.). Optionally, GP354 may be purified from the cells using standard chromatographic techniques. To facilitate purification, antisera are raised against one or more synthetic peptide sequences that correspond to portions of the GP354 amino acid sequence, and the antisera are used to affinity-purify GP354. The GP354 protein also may be expressed in-frame with a tag sequence (e.g., polyhistidine, haemagglutinin, or FLAG) to facilitate purification. Moreover, it will be appreciated that many of the uses for GP354 polypeptides, such as assays described below, do not require purification of GP354 from the host cell.

B. Expression of GP354 in 293 Cells

For expression of GP354 in mammalian cells 293 (transformed human or primate embryonic kidney cells), a plasmid bearing the relevant gp354 coding sequence is prepared, using vector pSecTag2A (Invitrogen). Vector pSecTag2A contains the murine IgK chain leader sequence for secretion, the c-myc epitope for detection of the recombinant protein with the anti-myc antibody, a C-terminal polyhistidine for purification with nickel chelate chromatography, and a Zeocin-resistant gene for selection of stable transfectants. The forward primer for amplification of this gp354 cDNA is determined by routine procedures and preferably contains a 5′ extension of nucleotides to introduce the HindIII cloning site and nucleotides matching the gp354 sequence. The reverse primer is also determined by routine procedures and preferably contains a 5′ extension of nucleotides to introduce an XhoI restriction site for cloning and nucleotides corresponding to the reverse complement of the gp354 sequence. The PCR conditions are 55° C. as the annealing temperature. The PCR product is gel purified and cloned into the HindIII-XhoI sites of the vector. The DNA is purified using QIAGEN chromatography columns and transfected into 293 cells using the DOTAP transfection medium (Boehringer Mannheim). Transiently transfected cells are tested for expression at 24 hours after transfection, using Western blots probed with anti-His and anti-GP354 peptide antibodies.

Permanently transfected cells are selected with Zeocin and propagated. Production of the recombinant protein is detected from both cells and media by Western blots probed with anti-His, anti-Myc or anti-GP354 peptide antibodies.

### C. Expression of GP354 in COS Cells

For expression of GP354 in COS7 cells, a polynucleotide having a sequence of SEQ ID NO:1, for example, can be cloned into vector p3-CI. This vector is a pUC 18-derived plasmid that contains the HCMV (human cytomegalovirus) promoter-intron located upstream from the bGH (bovine growth hormone) polyadenylation sequence and a multiple cloning site. In addition, the plasmid contains the dhrf (dihydrofolate reductase) gene which provides selection in the presence of the drug methotrexane (MTX) for selection of stable transformants.

The forward primer is determined by routine procedures and preferably contains a 5′ extension which introduces an XbaI restriction site for cloning, followed by nucleotides which correspond to a nucleotide sequence of SEQ ID NO:1. The reverse primer is also determined by routine procedures and preferably contains 5′-extension of nucleotides which introduces a SalI cloning site followed by nucleotides which correspond to the reverse complement of a nucleotide sequence of SEQ ID NO:1.

The PCR consists of an initial denaturation step of 5 min at 95° C.; 30 cycles of 30 sec denaturation at 95° C., 30 sec annealing at 58° C. and 30 sec extension at 72° C.; and followed by 5 min extension at 72° C. The PCR product is gel purified and ligated into the XbaI and SalI sites of vector p3-Cl. This construct is used to transform competent E. coli cells. The plasmid DNA is then purified from the E. coli culture with QIAGEN chromatography columns and transfected into COS7 cells using the LIPOFECTAMINE reagent from BRL in accordance with the manufacturer's specification. Forty-eight and 72 hours after transfection, the media and the cells are tested for recombinant protein expression.

GP354 expressed from a COS cell culture can be purified by first concentrating the cell-growth media to about 10 mg protein/ml. The purification can be accomplished by, for example, chromatography.

Purified GP354 is concentrated to 0.5 mg/ml in an AMICON concentrator fitted with a YM-10 membrane and stored at −80° C.

D. Expression of GP354 in Insect Cells

For expression of GP354 in a baculovirus system, a polynucleotide having a sequence of SEQ ID NO:1 is amplified by PCR. The forward primer is determined by routine procedures and preferably contains a 5′ extension which adds the NdeI cloning site, followed by nucleotides which correspond to a nucleotide sequence of SEQ ID NO:1. The reverse primer is also determined by routine procedures and preferably contains a 5′ extension which introduces the KpnI cloning site, followed by nucleotides which correspond to the reverse complement of a nucleotide sequence of SEQ ID NO:1.

The PCR product is gel purified, digested with NdeI and KpnI, and cloned into the corresponding sites of expression vector pAcHTL-A (Pharmingen, San Diego, Calif.). The pAcHTL vector contains the strong polyhedrin promoter of the Autographa californica nuclear polyhedrosis virus (AcMNPV), and a 6XHis tag upstream from the multiple cloning site. Nucleic acid sequences encoding a protein kinase site for phosphorylation and a thrombin site for excision of the recombinant protein precede the multiple cloning site.

Of course, many other baculovirus vectors, such as pAc373, pVL941 and pAcIM1, can be used in place of pAcHTL-A. Other suitable vectors for the expression of GP354 polypeptides can be also used, provided that the vector construct includes appropriately located signals for transcription, translation, and trafficking, such as an in-frame AUG and a signal peptide, as required. Such vectors are described in, e.g., Luckow et al., Virology 170:31-39 (1989).

The virus is grown and isolated using standard baculovirus expression methods, such as those described in Summers et al., A MANUAL OF METHODS FOR BACULOVIRUS VECTORS AND INSECT CELL CULTURE PROCEDURES, Texas Agricultural Experimental Station Bulletin No. 1555 (1987). In preferred embodiments, pAcHLT-A containing the gp354 gene is introduced into baculovirus using the BACULOGOLD transfection kit (Pharmingen). Individual virus isolates are analyzed for protein production by radiolabeling infected cells with ³⁵S-methionine at 24 hours post infection. Infected cells are harvested at 48 hours post infection, and the labeled proteins are visualized by SDS-PAGE. Viruses exhibiting high expression levels can be isolated and used for scaled up expression.

For expression of a GP354 polypeptide in a Sf9 cells, a polynucleotide having the sequence of SEQ ID NO:1 can be amplified by PCR using the methods described above for baculovirus expression. The gp354 cDNA is cloned into vector pAcHLT-A (Pharmingen) for expression in Sf9 insect cells. The insert is cloned into the NdeI and KpnI sites, after elimination of an internal NdeI site (using the same primers described above for expression in baculovirus). DNA is purified with QIAGEN chromatography columns and expressed in Sf9 cells. Preliminary Western blot experiments from non-purified plaques are tested for the presence of a recombinant protein of the expected size using a GP354-specific antibody. The results are confirmed after further purification and expression optimization in HiG5 cells.

### Example 10

### Interaction Trap/Two-hybrid System

In order to assay for GP354-interacting proteins, the interaction trap/two-hybrid library screening method can be used. This assay was first described in Fields et al., Nature 340:245 (1989). A protocol is published in CURRENT PROTOCOLS IN MOLECULAR BIOLOGY, John Wiley & Sons, NY (1999) and Ausubel, F. M. et al. SHORT PROTOCOLS IN MOLECULAR BIOLOGY, fourth edition, Greene and Wiley-interscience, NY (1992). Kits are commercially available from, e.g., Clontech (MATCHMAKER Two-Hybrid System 3).

A fusion of the nucleotide sequences encoding all or partial GP354 and the DNA-binding domain (DNA-BD) of yeast transcription factor GAL4 is constructed using an appropriate vector (i.e., pGBKT7). Similarly, a GAL4 active domain (AD) fusion library is constructed in a second plasmid (i.e., pGADT7) from cDNA of potential GP354-binding proteins. For protocols on making cDNA libraries, see, e.g., Sambrook et al. MOLECULAR CLONING: A LABORATORY MANUAL, second edition, Cold Spring Harbor Press, Cold Spring Harbor, N.Y. (1989).

The DNA-BD/GP354 fusion construct is verified by sequencing, and tested for autonomous reporter gene activation and cell toxicity, both of which would prevent a successful two-hybrid analysis. Similar controls are performed with the AD/library fusion construct to ensure expression in host cells and lack of transcriptional activity. Yeast cells are transformed (ca. 105 transformants/mg of DNA) with both the GP354 and library fusion plasmids according to standard procedure (Ausubel, et al., supra). In vivo binding of DNA-BD/GP354 with AD/library proteins results in transcription of specific yeast plasmid reporter genes (i.e., lacZ, HIS3, ADE2, LEU2). Yeast cells are plated on nutrient-deficient media to screen for expression of reporter genes. Colonies are dually assayed for b-galactosidase activity upon growth in Xga1 (5-bromo-4-chloro-3-indolyl-b-D-galactoside) supplemented media (filter assay for b-galactosidase activity is described in Breeden et al., Cold Spring Harb. Symp. Quant. Biol., 50:643 (1985). Positive AD-library plasmids are rescued from transformants and reintroduced into the original yeast strain as well as other strains containing unrelated DNA-BD fusion proteins to confirm specific GP354/library protein interactions. Insert DNA is sequenced to verify the presence of an open reading frame fused to GAL4 AD and to determine the identity of the GP354-binding protein.

### Example 11

### Antibodies To GP354 Polypeptides

Standard techniques are employed to generate polyclonal or monoclonal antibodies to GP354, and to generate useful antigen-binding fragments thereof or variants thereof, including “humanized” variants. Such protocols can be found, for example, in Sambrook et al., supra, and Harlow et al. (Eds.) supra). In some embodiments, recombinant GP354 polypeptides (or cells or cell membranes containing such polypeptides) are used as antigen to generate the antibodies. In other embodiments, one or more peptides having amino acid sequences corresponding to an immunogenic portion of GP354 (e.g., 6, 7, 8, 9, 10, 11, 12, 13, 14, 15, 16, 17, 18, 19, 20, or more amino acids) are used as antigen. Peptides corresponding to extracellular portions of GP354, especially hydrophilic extracellular portions, are preferred. The antigen may be mixed with an adjuvant or linked to a hapten to increase antibody production.

A. Polyclonal or Monoclonal Antibodies

In one exemplary protocol, recombinant GP354 or a synthetic fragment thereof is used to immunize a mouse to generate monoclonal antibodies, or to immunize a larger mammal, such as a rabbit, for polyclonal antibodies. To increase antigenicity, peptides can be conjugated to keyhole limpet hemocyanin commercially available from ,e.g., Pierce. For an initial injection, the antigen is emulsified with Freund's Complete Adjuvant and injected subcutaneously. At intervals of two to three weeks, additional aliquots of GP354 antigen are emulsified with Freund's Incomplete Adjuvant and injected subcutaneously. Prior to the final booster injection, a serum sample is taken from the immunized mice and assayed by Western blot to confirm the presence of antibodies that immunoreact with GP354. Sera from the immunized animals may be used as polyclonal antisera or used to isolate polyclonal antibodies that recognize GP354.

Alternatively, the mice are sacrificed and their spleen removed for generation of monoclonal antibodies. To generate monoclonal antibodies, the spleens are placed in 10 ml of serum-free RPMI 1640, and single cell suspensions are formed by grinding the spleens in serum-free RPMI 1640 supplemented with 2 mM L-glutamine, 1 mM sodium pyruvate, 100 units/ml penicillin, and 100 μg/ml streptomycin (RPMI) (Gibco, Canada). The cell suspensions are filtered and washed by centrifugation and resuspended in serum-free RPMI. Thymocytes taken from three naive Balb/c mice are prepared in a similar manner and used as a feeder layer. NS-1 myeloma cells, kept in log phase in RPMI with 10% fetal bovine serum (FBS) (Hyclone Laboratories, Inc., Logan, Utah) for three days prior to fusion, are centrifuged and washed as well.

To produce hybridoma fusions, spleen cells from the immunized mice are combined with NS-1 cells and centrifuged, and the supernatant is aspirated. The cell pellet is dislodged by tapping the tube, and 2 ml of 37° C. PEG 1500 (50% in 75 mM HEPES, pH 8.0) is stirred into the pellet, followed by the addition of serum-free RPMI. Thereafter, the cells are centrifuged, resuspended in RPMI containing 15% FBS, 100 μM sodium hypoxanthine, 0.4 μM aminopterin, 16 μM thymidine (HAT) (Gibco), 25 units/ml IL-6 (Boehringer-Mannheim) and 1.5×10⁶ thymocytes/ml, and plated into 10 flat-bottom 96-well tissue culture plates.

On days 2, 4, and 6 after the fusion, 100 μl of medium is removed from the wells of the tissue culture plates and replaced with fresh medium. On day 8, the fusions are screened by ELISA, testing for the presence of mouse IgG that binds to GP354. Cells from selected wells are further cloned by dilution until monoclonal cultures producing anti-GP354 antibodies are obtained.

B. Humanization of Anti-GP354 Monoclonal Antibodies

The expression pattern of GP354 as reported herein and the potential of GP354 as targets for therapeutic intervention suggest therapeutic indications for GP354 inhibitors (antagonists). GP354-neutralizing antibodies comprise one class of therapeutics useful as GP354 antagonists. The following are protocols to improve the utility of anti-GP354 monoclonal antibodies as therapeutics in humans by “humanizing” the monoclonal antibodies. Humanized antibodies have improved serum half-life and are less immunogenic in humans. The principles of antibody humanization have been described in the literature. For instance, to minimize potential binding to complement, a humanized antibody is preferred to be of the IgG₄ subtype.

One level of humanization can be achieved by generating chimeric antibodies comprising the variable domains of a non-human antibody of interest and the constant domains of a human antibody. See, e.g., Morrison et al., Adv. Immunol., 44:65-92 (1989). The variable domains of anti-GP354 antibodies can be cloned from the genomic DNA of an appropriate B-cell hybridoma or from cDNA derived from the hybridoma. The V region gene fragments are linked to exons encoding human antibody constant domains. The resultant construct is expressed in suitable mammalian host cells (e.g., myeloma or CHO cells).

To achieve an even greater level of humanization, only those portions of the variable region gene fragments that encode antigen-binding complementarity determining regions (CDRs) of the non-human monoclonal antibody are cloned into human antibody sequences. See, e.g., Jones et al., Nature 321:522-525 (1986); Riechmann et al., Nature 332:323-327 (1988); Verhoeyen et al., Science 239:1534-36 (1988); and Tempest et al., Bio/Technology 9:266-71 (1991). If necessary, the β-sheet framework of the human antibody surrounding the CDR3 region is also modified (i.e., “back-mutated”) to more closely mirror the three dimensional structure of the antigen-binding site of the original monoclonal antibody. See Kettleborough et al., Protein Engin. 4:773-783 (1991); and Foote et al., J. Mol. Biol. 224:487-499 (1992).

In an alternative approach, the surface of a non-human monoclonal antibody of interest is humanized by altering selected surface residues of the non-human antibody, e.g., by site-directed mutagenesis, while retaining all of the interior and contacting residues of the non-human antibody. See Padlan, Mol. Immunol., 28(4/5):489-98 (1991).

The foregoing approaches are employed using anti-GP354 monoclonal antibodies and the hybridomas that produce them. The humanized anti-GP354 antibodies are useful as therapeutics to treat or palliate conditions wherein GP354 expression or ligand-mediated GP354 signaling is undesirable.

C. Human GP354-neutralizing Antibodies from Phage Display

Anti-GP354 antibodies can be also generated by phage display techniques such as those described in Aujame et al., Hum. Antibodies 8(4):155-168 (1997); Hoogenboom, supra; and Rader et al., Curr. Opin. Biotechnol. 8:503-508 (1997). For example, antibody variable regions in the form of Fab fragments or linked single chain Fv fragments are fused to the amino terminus of filamentous phage minor coat protein pIII. Expression of the fusion protein and incorporation thereof into the mature phage coat results in phage particles that present an antibody on their surface and contain the genetic material encoding the antibody. A phage library comprising such constructs is expressed in bacteria, and the library is screened for GP354-specific phage-antibodies using labeled or immobilized GP354 as antigen-probe.

D. Human GP354-specific Antibodies from Transgenic Mice

Human GP354-specific antibodies are generated in transgenic mice essentially as described in Brüggemann et al., Immunol. Today 17(8):391-97 (1996) and Bruggemann et al., Curr. Opin. Biotechnol. 8:455-58 (1997). Transgenic mice carrying human V-gene segments in germline configuration and that express these transgenes in their lymphoid tissue are immunized with a GP354 composition using conventional immunization protocols. Hybridomas are generated using B cells from the immunized mice using conventional protocols and screened to identify hybridomas secreting anti-GP354 human antibodies (e.g., as described above).

### Example 12

### Assays to Identify Modulators of GP354 Activity

Set forth below are several non-limiting assays for identifying modulators (agonists and antagonists) of GP354 activity. Among the modulators that can be identified by these assays are natural ligands of the receptor; synthetic analogs and derivatives of the natural ligands; antibodies and/or antibody-like compounds derived from natural antibodies or from antibody-like combinatorial libraries; and/or synthetic compounds identified by high-throughput screening of libraries; and the like.

All modulators that bind GP354 are useful for identifying GP354 in tissue samples (e.g., for diagnostic purposes or therapeutic purposes). Agonist and antagonist modulators are useful for up-regulating and down-regulating GP354 activity, respectively, so as to treat GP354-mediated diseases. The assays may be performed using single putative modulators, and/or may be performed using a known agonist in combination with candidate antagonists (or visa versa).

A. cAMP Assays

In one type of assay, levels of cyclic adenosine monophosphate (cAMP) are measured in gp354-transfected cells that have been exposed to candidate modulator compounds. Protocols for cAMP assays have been described in the literature. See, e.g., Sutherland et al., Circulation 37:279 (1968); Frandsen et al., Life Sci. 18:529-541 (1976); Dooley et al., J. of Pharmacol. Exp. Therap. 283(2): 735-41 (1997); and George et al., J. Biomol. Screening 2(4):235-40 (1997). An exemplary protocol for such an assay, using an Adenylyl Cyclase Activation FLASHPLATE Assay from NEN Life Science Products, is set forth below.

Briefly, a GP354-encoding sequence is subcloned into an expression vector, such as pzeoSV2 (Invitrogen). CHO cells are transiently transfected with the resultant expression construct using known methods, such as the transfection protocol provided by Boehringer-Mannheim when supplying the FUGENE 6 transfection reagent. Transfected CHO cells are seeded into 96-well microplates from the FLASHPLATE assay kit, which are coated with solid scintillant to which antisera to cAMP have been bound. For a control, some wells are seeded with untransfected CHO cells. Other wells in the plate receive various amounts of a cAMP standard solution for use in creating a standard curve. One or more test compounds are added to the cells in each well, with compound-free medium or buffer as control. After treatment, cAMP is allowed to accumulate in the cells for exactly 15 minutes at room temperature. The assay is terminated by the addition of lysis buffer containing [¹²⁵]-cAMP, and the plate is counted using a Packard TOPCOUNT 96-well microplate scintillation counter. Unlabeled cAMP from the lysed cells or from standards and fixed amounts of [¹²⁵I]-cAMP compete for antibody bound to the plate. A standard curve is constructed, and cAMP values for the unknowns are obtained by interpolation. Changes in intracellular cAMP levels of cells in response to exposure to a test compound are indicative of GP354 modulating activity. Modulators that act as agonists of receptors which couple to the Gs subtype of G proteins will stimulate production of cAMP, leading to a measurable (e.g., 3-10) fold increase in cAMP levels. Agonists of receptors which couple to the Gi/o subtype of G proteins will inhibit forskolin-stimulated cAMP production, leading to a measurable decrease (e.g., 50-100%) in cAMP levels. Modulators that act as inverse agonists will reverse these effects at receptors that are either constitutively active or activated by known agonists.

B. Aequorin Assays

In another assay, cells (e.g., CHO cells) are transiently co-transfected with a gp354 expression construct and a construct that encodes the photoprotein apoaquorin. In the presence of the cofactor coelenterazine, apoaquorin will emit a measurable luminescence that is proportional to the amount of cytoplasmic free calcium. See generally, Cobbold, et al. “Aequorin measurements of cytoplasmic free calcium,” In: McCormack J. G. and Cobbold P. H., eds., CELLULAR CALCIUM: A PRACTICAL APPROACH. Oxford:IRL Press (1991); Stables et al., Anal. Biochem. 252:115-26 (1997); and Haugland, HANDBOOK OF FLUORESCENT PROBES AND RESEARCH CHEMICALS, Sixth edition, Eugene Oreg. (1996).

In one exemplary assay, a gp354 coding sequence is subcloned into pzeoSV2 (Invitrogen). CHO cells are transiently co-transfected with the resultant expression construct and a construct that encodes the photoprotein apoaquorin (Molecular Probes) using the transfection reagent FUGENE 6 (Boehringer-Mannheim) and the transfection protocol provided in the product insert.

The cells are cultured for 24 hours at 37° C. in MEM (Gibco/BRL, Gaithersburg, MD) supplemented with 10% fetal bovine serum, 2 mM glutamine, 10 U/ml penicillin and 10 μg/ml streptomycin. Then the culture medium is changed to serum-free MEM containing 5 μM coelenterazine (Molecular Probes). Culturing is continued for two more hours at 37° C. Subsequently, the cells are detached from the plate using VERSEN (Gibco/BRL), washed, and resuspended at 2×10⁻⁵ cells/ml in serum-free MEM.

Dilutions of candidate GP354 modulator compounds are prepared in serum-free MEM and dispensed into wells of an opaque 96-well assay plate at 50 μl/well. The plate is then loaded onto an MLX microtiter plate luminometer (Dynex Technologies, Inc., Chantilly, Va.). The instrument is programmed to dispense 50 μl cell suspensions into each well, one well at a time, and immediately read luminescence for 15 seconds. Dose-response curves for the candidate modulators are constructed using the area under the curve for each light signal peak. Data are analyzed with SLIDEWRITE, using the equation for a one-site ligand, and EC50 values are obtained. Changes in luminescence caused by the compounds are considered indicative of modulatory activity. Modulators that act as agonists at receptors which couple to the Gq subtype of G proteins give an increase in luminescence of up to 100 fold. Modulators that act as inverse agonists will reverse this effect at receptors that are either constitutively active or activated by known agonists.

C. Luciferase Reporter Gene Assay

The photoprotein luciferase provides another useful tool for identifying GP354 modulators. Cells (e.g., CHO cells or COS7 cells) are transiently co-transfected with a gp354 expression construct and a reporter construct which includes a gene for the luciferase protein downstream from a transcription factor binding site, such as the cAMP-response element (CRE), AP-1, or NF-kappa B. Expression levels of luciferase reflect the activation status of the signaling events. See generally, George et al., J. Biomol. Screening 2(4):235-240 (1997); and Stratowa et al., Curr. Opin. Biotechnol. 6:574-581 (1995). Luciferase activity may be quantitatively measured using, e.g., luciferase assay reagents that are available from Promega (Madison, Wis.).

In one exemplary assay, CHO cells are plated in 24-well culture plates at a density of 10⁵ cells/well one day prior to transfection, and cultured at 37° C. in MEM (Gibco/BRL) supplemented with 10% fetal bovine serum, 2 mM glutamine, 10 U/ml penicillin and 10 μg/ml streptomycin. Cells are transiently co-transfected with a gp354 expression construct and a reporter construct containing the luciferase gene. The reporter plasmid constructs CRE-luciferase, AP-1-luciferase and NF-kappaB-luciferase may be purchased from Stratagene (LaJolla, Calif.). Transfections are performed using the FUGENE 6 transfection reagent (Boehringer-Mannheim) according to the supplier's instructions. Cells transfected with the reporter construct alone are used as a control.

Twenty-four hours after transfection, the cells are washed once with PBS pre-warmed to 37° C. Serum-free MEM is then added to the cells either alone (control) or with one or more candidate modulators. The cells are then incubated at 37° C. for five hours. Thereafter, the cells are washed once with ice-cold PBS and lysed by the addition of 100 μl of lysis buffer per well from the luciferase assay kit supplied by Promega. After incubation for 15 minutes at room temperature, 15 μl of the lysate is mixed with 50 μl of substrate solution (Promega) in an opaque-white, 96-well plate, and the luminescence is read immediately on a Wallace model 1450 MICROBETA scintillation and luminescence counter (Wallace Instruments, Gaithersburg, Md.).

Differences in luminescence in the presence versus the absence of a candidate modulator compound are indicative of modulatory activity. Receptors that are either constitutively active or activated by agonists typically give a 3-fold to 20-fold stimulation of luminescence compared to cells transfected with the reporter gene alone. Modulators that act as inverse agonists will reverse this effect.

D. Intracellular Calcium Measurement Using FLIPR

Changes in intracellular calcium levels are another recognized indicator of receptor activity, and such assays can be employed to screen for modulators of GP354 activity. For example, CHO cells stably transfected with a gp354 expression vector are plated at a density of 4×10⁴ cells/well in Packard black-walled, 96-well plates specially designed to discriminate fluorescence signals emanating from the various wells on the plate. The cells are incubated for 60 minutes at 37° C. in modified Dulbecco's PBS (D-PBS) containing 36 mg/L pyruvate and 1 g/L glucose with the addition of 1% fetal bovine serum and one of four calcium indicator dyes (FLUO-3 AM, FLUO-4 AM, CALCIUM GREEN-1 AM, or OREGON GREEN 488 BAPTA-1 AM), each at a concentration of 4 μM. Plates are washed once with modified D-PBS without 1% fetal bovine serum and incubated for 10 minutes at 37° C. to remove residual dye from the cellular membrane. In addition, a series of washes with modified D-PBS without 1% fetal bovine serum is performed immediately prior to activation of the calcium response.

A calcium response is initiated by the addition of one or more candidate receptor agonist compounds, calcium ionophore A23187 (10 μM; positive control), or ATP (4 μM; positive control). Fluorescence is measured by Molecular Device's FLIPR with an argon laser (excitation at 488 nm). See, e.g., Kuntzweiler et al., Drug Dev. Res. 44(1): 14-20 (1998). The F-stop for the detector camera is set at 2.5 and the length of exposure is 0.4 milliseconds. Basal fluorescence of cells is measured for 20 seconds prior to addition of a candidate agonist, ATP, or A23187. The basal fluorescence level is subtracted from the response signal. The calcium signal is measured for approximately 200 seconds, taking readings every two seconds. Calcium ionophore A23187 and ATP typically increase the calcium signal about 200% above baseline levels. In general, activated GP354s increase the calcium signal at least about 10-15% above baseline signal.

E. Mitogenesis Assay

In a mitogenesis assay, the ability of candidate modulators to induce or inhibit gp354-mediated cell division is determined. See, e.g., Lajiness et al., J. Pharmacol. Exp. Therap. 267(3):1573-1581 (1993). For example, CHO cells stably expressing GP354 are seeded into 96-well plates at a density of 5000 cells/well and grown at 37° C. in MEM with 10% fetal calf serum for 48 hours, at which time the cells are rinsed twice with serum-free MEM. After rinsing, 80 μl of fresh MEM, or MEM containing a known mitogen, is added along with 20 μl MEM containing varying concentrations of one or more test compounds diluted in serum-free medium. As controls, some wells on each plate receive serum-free medium alone, and some receive medium containing 10% fetal bovine serum. Untransfected cells or cells transfected with vector alone also may serve as controls.

After culture for 16-18 hours, 1 μCi of [³H]-thymidine (2 Ci/mmol) is added to the wells and cells are incubated for an additional 2 hours at 37° C. The cells are trypsinized and collected on filter mats with a cell harvester (Tomtec); the filters are then counted in a Betaplate counter. The incorporation of [³H]-thymidine in serum-free test wells is compared to the results achieved in cells stimulated with serum (positive control). Use of multiple concentrations of test compounds permits creation and analysis of dose-response curves using the non-linear, least squares fit equation: A=B×[C/(D+C)]+G where A is the percent of serum stimulation; B is the maximal effect minus baseline; C is the EC50; D is the concentration of the compound; and G is the maximal effect. Parameters B, C and G are determined by Simplex optimization.

Agonists that bind to the receptor are expected to increase [³H]-thymidine incorporation into cells, showing up to 80% of the response to serum. Antagonists that bind to the receptor will inhibit the stimulation seen with a known agonist by up to 100%.

F. [³⁵S]GTPgS Binding Assay

It is possible to evaluate whether GP354 signals through a G protein-mediated pathway. G protein-coupled receptors signal through intracellular G proteins whose activities involve GTP binding and hydrolysis to yield bound GDP. Thus, measurement of binding of the non-hydrolyzable GTP analog [³⁵S]GTPgS in the presence and absence of candidate modulators provides another assay for modulator activity. See, e.g., Kowal et al., Neuropharmacology 37:179-187 (1998).

In one exemplary assay, cells stably transfected with a gp354 expression vector are grown in 10 cm tissue culture dishes to subconfluence, rinsed once with 5 ml of ice-cold Ca²⁺/Mg²⁺-free phosphate-buffered saline, and scraped into 5 ml of the same buffer. Cells are pelleted by centrifugation (500×g, 5 minutes), resuspended in TEE buffer (25 mM Tris, pH 7.5, 5 mM EDTA, 5 mM EGTA), and frozen in liquid nitrogen. After thawing, the cells are homogenized using a Dounce homogenizer (1 ml TEE per plate of cells), and centrifuged at 1,000×g for 5 minutes to remove nuclei and unbroken cells.

The homogenate supernatant is centrifuged at 20,000×g for 20 minutes to isolate the membrane fraction, and the membrane pellet is washed once with TEE and resuspended in binding buffer (20 mM HEPES, pH 7.5, 150 mM NaCl, 10 mM MgCl2, 1 mM EDTA). The resuspended membranes can be frozen in liquid nitrogen and stored at −70° C. until use.

Aliquots of cell membranes prepared as described above and stored at −70° C. are thawed, homogenized, and diluted into buffer containing 20 mM HEPES, 10 mM MgCl2, 1 mM EDTA, 120 mM NaCl, 10 μM GDP, and 0.2 mM ascorbate, at a concentration of 10-50 μg/ml. In a final volume of 90 μl, homogenates are incubated with varying concentrations of candidate modulator compounds or 100 μM GTP for 30 minutes at 30° C. and then placed on ice. To each sample, 10 μl guanosine 5′-O-(3[³⁵S]thio) triphosphate (NEN, 1200 Ci/mmol; [³⁵S]-GTPgS), was added to a final concentration of 100-200 pM. Samples are incubated at 30° C. for an additional 30 minutes, 1 ml of 10 mM HEPES, pH 7.4, 10 mM MgCl2, at 4° C. is added and the reaction is stopped by filtration.

Samples are filtered over Whatman GF/B filters and the filters are washed with 20 ml ice-cold 10 mM HEPES, pH 7.4, 10 mM MgCl₂. Filters are counted by liquid scintillation spectroscopy. Nonspecific binding of [³⁵S]-GTPgS is measured in the presence of 100 μM GTP and subtracted from the total. Compounds are selected that modulate the amount of [³⁵S]-GTPgS binding in the cells, compared to untransfected control cells. Activation of receptors by agonists gives up to a five-fold increase in [³⁵S]-GTPgS binding. This response is blocked by antagonists.

G. MAP Kinase Activity Assay

Evaluation of MAP kinase activity in cells expressing GP354 provides another assay to identify modulators of GP354 activity. See, e.g., Lajiness et al., supra, and Boulton et al., Cell 65:663-675 (1991). In one embodiment, CHO cells stably transfected with gp354 are seeded into 6-well plates at a density of 7×10⁴ cells/well 48 hours prior to the assay. During this 48 hour period, the cells are cultured at 37° C. in MEM medium supplemented with 10% fetal bovine serum, 2 mM glutamine, 10 U/ml penicillin and 10 μg/ml streptomycin. The cells are serum-starved for 1-2 hours prior to the addition of stimulants.

For the assay, the cells are treated with medium alone or medium containing either a candidate agonist or 200 nM Phorbol ester-myristoyl acetate (i.e., PMA, a positive control), and the cells are incubated at 37° C. for various amounts of time. To stop the reaction, the plates are placed on ice, the medium is aspirated, and the cells are rinsed with 1 ml of ice-cold PBS containing 1 mM EDTA. Thereafter, 200 μl of cell lysis buffer (12.5 mM MOPS, pH 7.3, 12.5 mM glycerophosphate, 7.5 mM MgCl₂, 0.5 mM EGTA, 0.5 mM sodium vanadate, 1 mM benzamidine, 1 mM dithiothreitol, 10 μg/ml leupeptin, 10 μg/ml aprotinin, 2 μg/ml pepstatin A, and 1 μM okadaic acid) is added to the cells. The cells are scraped from the plates and homogenized by 10 passages through a 23 ¾ G needle, and the cytosol fraction is prepared by centrifugation at 20,000×g for 15 minutes.

Aliquots (5-10 μl containing 1-5 μg protein) of cytosol are mixed with 1 mM MAPK Substrate Peptide (APRTPGGRR (SEQ ID NO:18), Upstate Biotechnology, Inc., NY) and 50 μM [g-³²P]ATP (NEN, 3000 Ci/mmol), diluted to a final specific activity of about 2000 cpm/pmol, in a total volume of 25 μl. The samples are incubated for 5 minutes at 30° C., and reactions are stopped by spotting 20 μl on 2 cm² squares of Whatman P81 phosphocellulose paper. The filter squares are washed in 4 changes of 1% H₃PO₄, and the squares are subjected to liquid scintillation spectroscopy to quantitate bound label. Equivalent cytosolic extracts are incubated without MAPK substrate peptide, and the bound labels from these samples are subtracted from the matched samples with the substrate peptide. The cytosolic extract from each well is used as a separate point. Protein concentrations are determined by a dye binding protein assay (Bio-Rad Laboratories). Agonist activation of the receptor is expected to result in up to a five-fold increase in MAPK enzyme activity. This increase is blocked by antagonists.

H. [³H]Arachidonic Acid Release

The activation of GP354s may also potentiate arachidonic acid release in cells, providing yet another useful assay for modulators of GP354 activity. See, e.g., Kanterman et al., Mol. Pharmacol. 39:364-369 (1991). For example, CHO cells that are stably transfected with a GP354 expression vector are plated in 24-well plates at a density of 1.5×10⁴ cells/well and grown in MEM medium supplemented with 10% fetal bovine serum, 2 mM glutamine, 10 U/ml penicillin and 10 μg/ml streptomycin for 48 hours at 37° C. before use. Cells of each well are labeled by incubation with [³H]-arachidonic acid (Amersham Corp., 210 Ci/mmol) at 0.5 μCi/ml in 1 ml MEM supplemented with 10 mM HEPES, pH 7.5, and 0.5% fatty-acid-free bovine serum albumin for 2 hours at 37° C. The cells are then washed twice with 1 ml of the same buffer. Candidate compounds are added in 1 ml of the same buffer, either alone or with 10 μM ATP, and the cells are incubated at 37° C. for 30 minutes. Buffer alone and mock-transfected cells are used as controls. Samples (0.5 ml) from each well are counted by liquid scintillation spectroscopy. Agonists which activate the receptor will lead to potentiation of the ATP-stimulated release of [³H]-arachidonic acid. This potentiation is blocked by antagonists.

I. Extracellular Acidification Rate

In yet another assay, the effects of candidate modulators of GP354 activity are assayed by monitoring extracellular changes in pH induced by the test compounds. See, e.g., Dunlop et al., J. Pharmacol. Toxicol. Meth. 40(1):47-55 (1998). In one embodiment, CHO cells transfected with a GP354 expression vector are seeded into 12 mm capsule cups (Molecular Devices Corp.) at 4×10⁵ cells/cup in MEM supplemented with 10% fetal bovine serum, 2 mM L-glutamine, 10 U/ml penicillin, and 10 μg/ml streptomycin. The cells are incubated in this medium at 37° C. in 5% CO2 for 24 hours.

Extracellular acidification rates are measured using a CYTOSENSOR MICROPHYSIOMETER (Molecular Devices Corp.). The capsule cups are loaded into the sensor chambers of the MICROPHYSIOMETER and the chambers are perfused with running buffer (bicarbonate-free MEM supplemented with 4 mM L-glutamine, 10 units/ml penicillin, 10 μg/ml streptomycin, 26 mM NaCl) at a flow rate of 100 μl/min. Candidate agonists or other agents are diluted into the running buffer and perfused through a second fluid path. During each 60-second pump cycle, the pump is run for 38 seconds and is off for the remaining 22 seconds. The pH of the running buffer in the sensor chamber is recorded during the cycle from 43-58 seconds, and the pump is re-started at 60 seconds to start the next cycle. The rate of acidification of the running buffer during the recording time is calculated by the Cytosoft program. Changes in the rate of acidification are calculated by subtracting the baseline value (the average of 4 rate measurements immediately before addition of a modulator candidate) from the highest rate measurement obtained after addition of a modulator candidate. The selected instrument detects 61 mV/pH unit. Modulators that act as agonists of the receptor result in an increase in the rate of extracellular acidification compared to the rate in the absence of agonist. This response is blocked by modulators which act as antagonists of the receptor.

### Example 13

### Cloning of Other gp354 Genes or Fragments

To obtain other gp354 genes or fragments, PCR was performed essentially as described in Example 3, supra. Oligonucleotides GX1-220 and GX1-221 were used for inter-exon PCR verification of the ab initio gp354 gene model. GX1-386 and GX1-387 were used to amplify the full extent of the predicted portion of the gene. GX1-218 and GX1-221 were used to amplify and clone the mouse PanCAM orthologue. GX3-238 and GX3-232 were used to amplify the full length PanCAM cDNA from a pancreas cDNA pool. Their sequences are as following:

| | | |
|---|---|---|
| GX1-218: | ACTGGGGGCTAGTTCAGTGGACTAA | (SEQ ID NO:16) |
| GX1-220: | GCCAGCATGACCTCCACATTAG | (SEQ ID NO:19) |
| GX1-221: | CAGCCTCAGCTCTGCACACATAGTC | (SEQ ID NO:20) |
| GX1-386: | ATGCGGGTCCCCGCCCTCCTCGTCCT | (SEQ ID NO:21) |
| GX1-387: | CTTGGTCTCCAGAGTCCCTTCCTCCTC | (SEQ ID NO:22) |
| GX3-232: | GGAGGAGTAAGGTCGCCAACTTGTC | (SEQ ID NO:23) |
| GX3-238: | ACCTTGGGGGACGAATGCTC | (SEQ ID NO:24) |

The PCR products were isolated and cloned as described in Example 3, supra.

The cloned GX1-220/GX1-221 PCR product was labeled by random hexamer extension using the protocol provided with the random hexamer kit (Invitrogen, Carlsbad, Calif.). The labeled fragment was used as a probe in Northern and multiple tissue expression (MTE) blots. Hybridizations were done according to the protocol provided with the multiple tissue blots (Clontech).

The Northern blot shows a diffused signal detected only in the pancreas, with a size in the range of 2.2-3.5 Kb. Hybridization done with an array of 76 normal human tissues revealed a positive spot in the position of the pancreas RNA sample. These data confirm that the gp354 gene is predominantly expressed in the pancreas.

### Example 14

### Cloning of the Full Length Human gp354 Gene

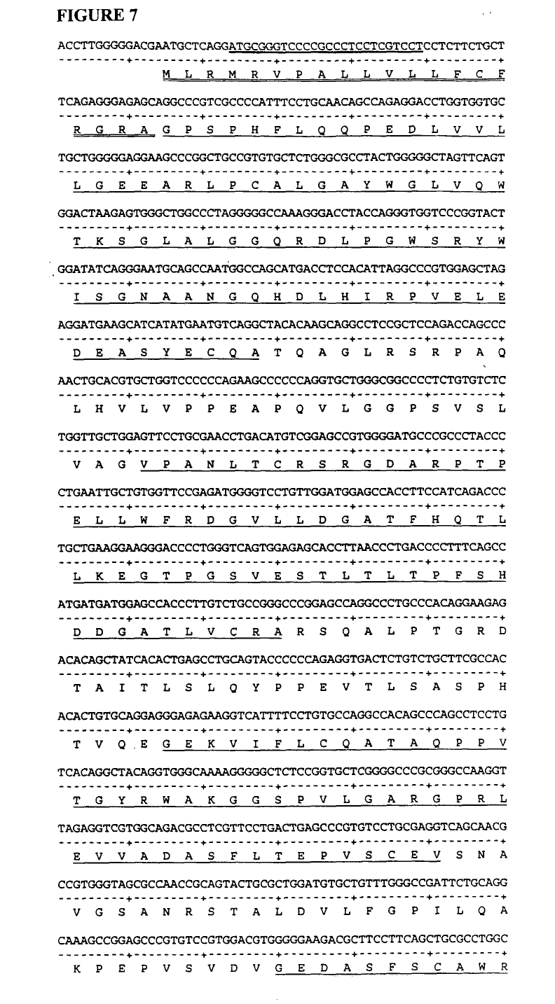

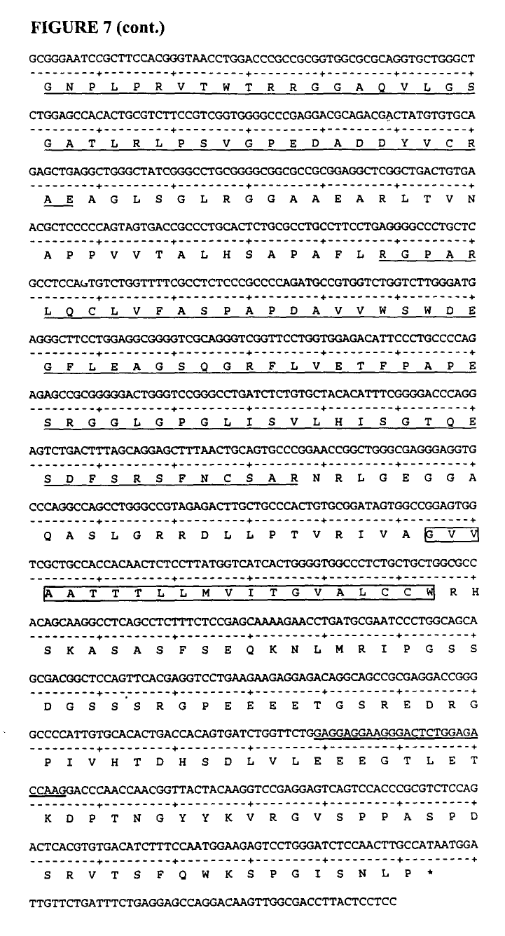

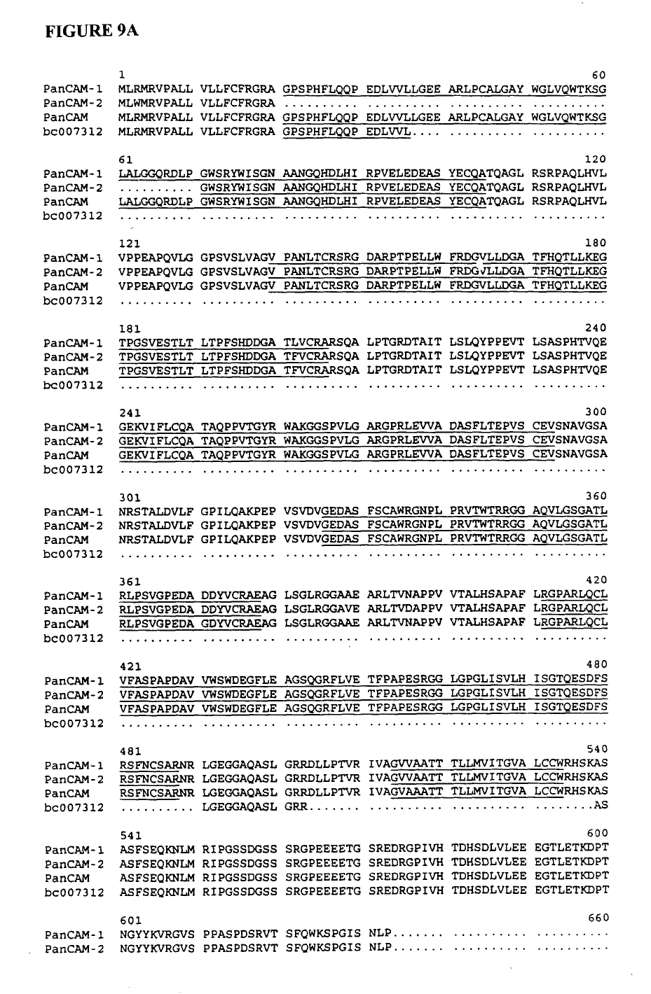

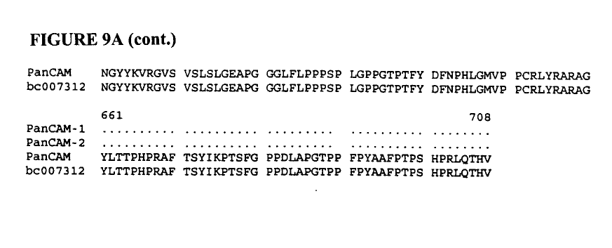

The entire 1,779 (including the stop codon) base pair predicted portion of the gene was amplified from a human pancreas cDNA pool by PCR, using primers GX1-386 and GX1-387 (supra) that encompassed the full length of the predicted open reading frame (FIG. 7). To amplify a full-length cDNA, we designed a pair of oligonucleotides (GX1-238 and GX1-232, supra) in the presumed 5′ and 3′ un-translated regions of the AL136654 sequence and used these for PCR of a human pancreas cDNA pool. Two different cDNA clones were obtained. Both were smaller than AL136654 and were within the size range determined on the Northern blot. They had uninterrupted open reading frames similar to that obtained from the original gene prediction, and encode proteins with 4 or 5 Ig domains (FIG. 9A).

Since this gene is predominantly expressed in the pancreas and encodes a protein with multiple C2 type Ig domains that are characteristic for cell adhesion molecules (Williams et al., Annu. Rev. Immunol. 6:381-405 (1988)), we also named this gene PanCAM, for pancreatic cell adhesion molecule.

The predicted protein encoded by the longest potential ORF of PanCAM is composed of 708 amino acid residues and contains a signal peptide, five Ig domains (three of them are C2 type), a trasmembrane domain, and a 174 amino acid intracellular domain. It is longer than the protein encoded by our original gene prediction on both the amino and carboxy termini.

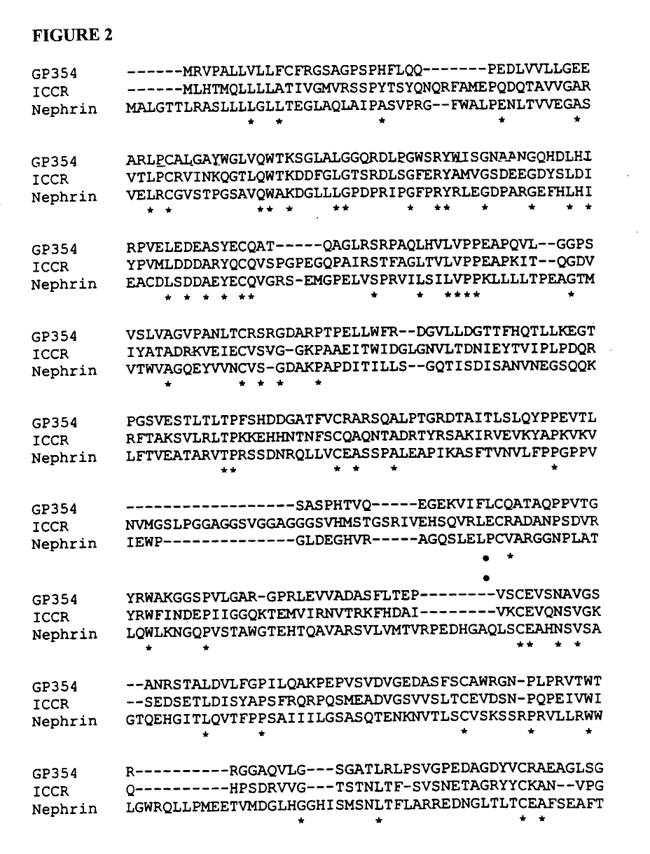

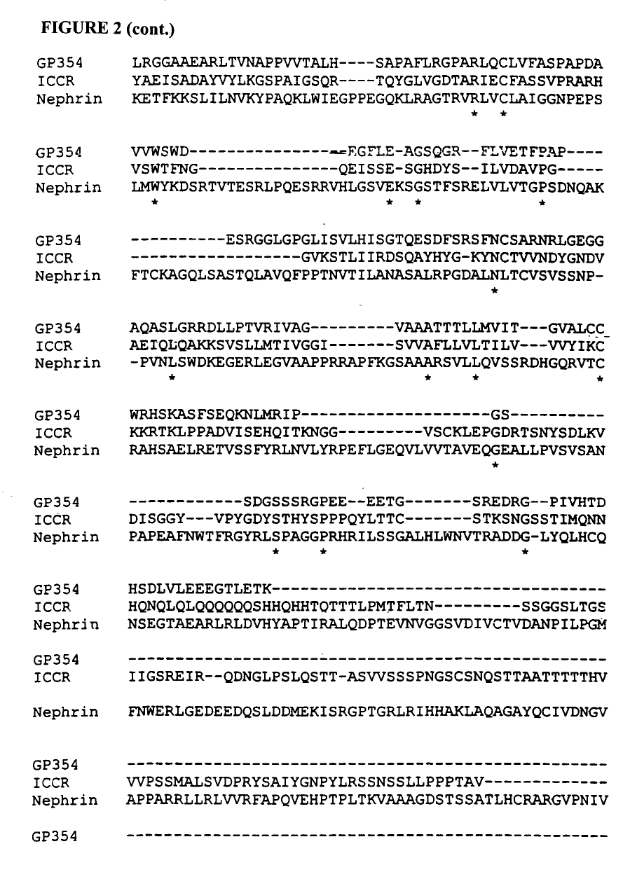

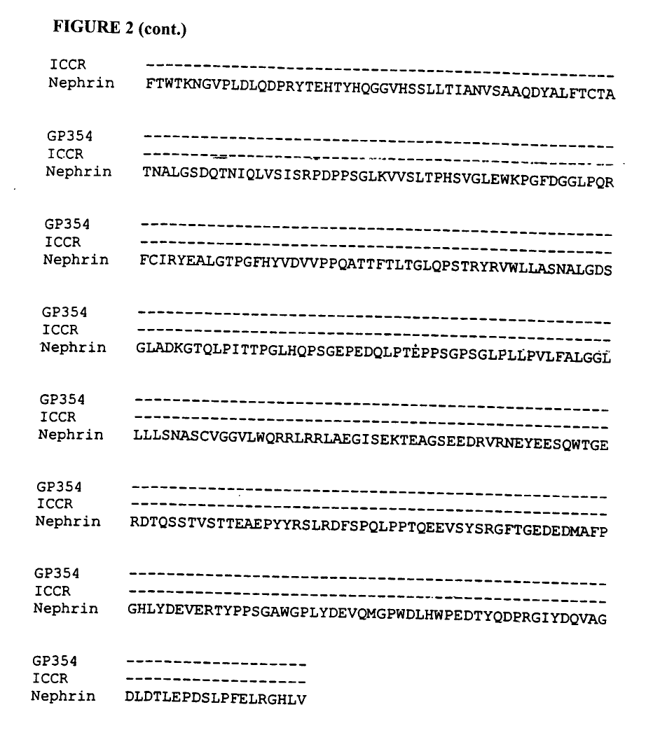

BLASTP searches of public databases identified several proteins with significant similarity to PanCAM. The three most similar proteins that have been characterized are human and mouse NEPH1, nephrin, and the Drosophila irregular chiasm-C roughest (“Irre”) protein (see, e.g., FIG. 2). In all of these cases, the homology is concentrated in the extracellular portion of the protein and there is no significant similarity in the intracellular portion.

Human NEPH1 (NP_{—}060710) and mouse NEPH1 proteins (NP_{—}570937) are potential cell adhesion molecules that play critical roles in kidney development (Donoviel et al., Mol. Cell. Biol. 21(14):4829-36 (2001)). Like PanCAM, NEPH1 has 5 Ig domains, and the human protein is 44% identical to PanCAM in the extracellular domain, while the mouse protein is 47% identical to PanCAM in the extracellular domain.

The second most similar BLASTP score is for human nephrin (NPHS1, NP_{—}004637), which shares 33% identity with GP354 in the extracellular domain. This protein contains eight Ig domains and is localized to the glomerula slit diaphragm. Nepgrin is believed to play a role in cell adhesion and to be involved in the development of glomeruli of kidney (Ruotsalainen et al., Proc. Natl. Acad. Sci. USA 96:7962-7967 (1999); Kestila et al., Mol. Cell 1(4):575-82 (1998)). Mutations in the nephrin gene cause congenital nephritis in humans (Kestila et al., supra).

The Drosophila irregular chiasm C roughest precursor (irre, NP_{—}525058) is another homologous protein. Irre is a cell adhesion protein required for development of compound eyes (Ramos et al., Genes Dev. 7(12B):2533-47 (1993); Schneider et al., Neuron 15(2):259-71 (1995)) and antenna (Reddy et al., Dev. Genes Evol. 209(10):581-91 (1999)). This protein contains 5 C2 type Ig domains, and shares 31% identity with PanCAM. Mutations at the Irre locus in Drosophila affect sensory organ development in the fly, apparently due at least in part to abnormal apoptotic activity (Ramos et al., supra).

NEPH1, nephrin and Irre are involved in developmental patterning and cell-cell communication. The similarity between GP354 and these proteins suggests that GP354 also plays a role in similar developmental pathways and, in particular, cell-cell interactions which trigger signal transduction pathways involved in organ and tissue development and/or maintenance in the pancreas and nervous system.

### Example 15

### Alternative Splicing of PanCAM

Sequence comparison of the two pancreas cDNA clones with two public EST sequences, AL136654 and BC007312 indicates that there are at least 4 alternative splice products of PanCAM (FIG. 9B). AL136654, identified in a fetal brain cDNA library, encompasses all of the predicted exons and most likely represents the longest full-length cDNA. BC007312, an EST clone derived from retinoblastoma cDNA, is the shortest transcript of PanCAM identified so far. BC007312 is generated by using cryptic splicing sites in exon 2 and exon 11 (FIG. 9B), where the encoded protein lacks all of the Ig domains, and has the signal peptide fused to the putative intracellular domain.

The pancreas cDNA clones differ from one another in the extracellular domain, but have identical transmembrane and intracellular domains. The shorter cDNA clone (PanCAM-2) from pancreas encodes a protein that is missing part of Ig domain 1, due to splicing out of exon 2 (FIG. 9B). The intracellular domains of the two pancreas cDNA clones are shorter than that of the other inferred protein sequences (FIG. 9A) due to splicing out part of the last exon.

### Example 16

### Cloning a partial cDNA of mouse PanCAM

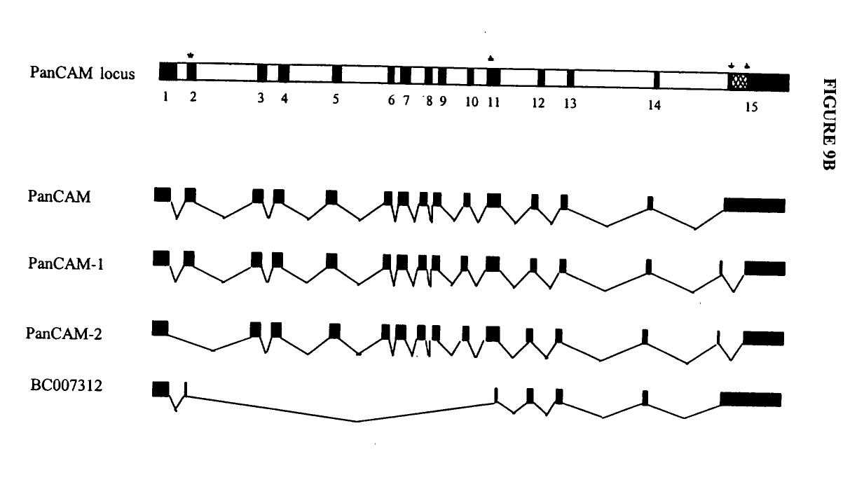

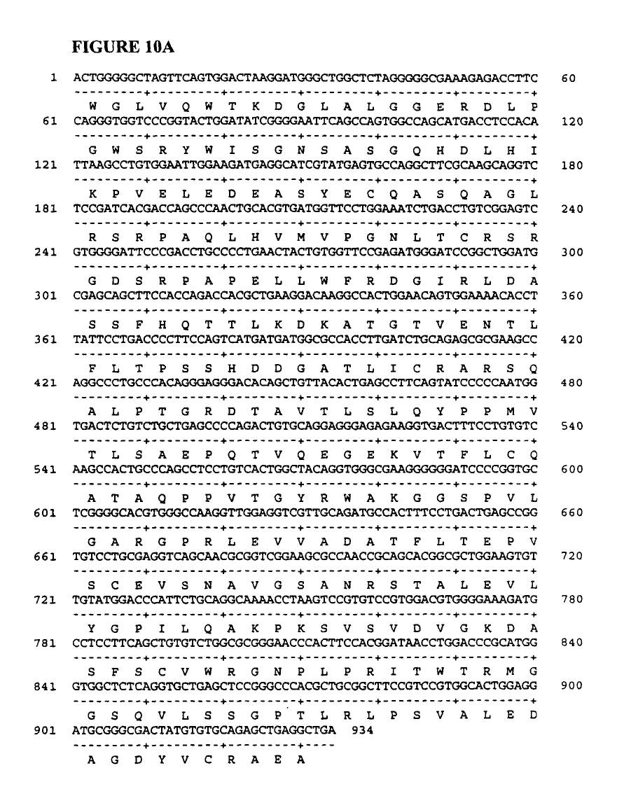

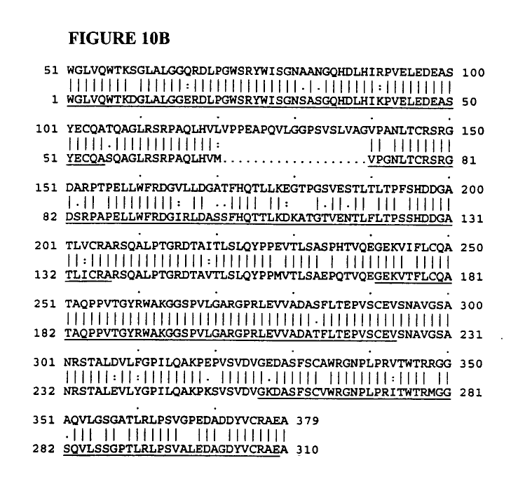

To detect and clone the mouse ortholog of PanCAM, multiple inter-exon oligonucleotide pairs were designed based on the human gene sequence, and tested by PCR on cDNA pools from multiple mouse tissues. One oligonucleotide pair generated a faint band from a mouse brain cDNA pool. This PCR product was cloned and sequenced. The mouse cDNA clone (SEQ ID NO:32) was 934 nucleotides in length, and the encoded protein is 85% identical to the human counterpart (FIGS. 10A-B).

### Example 17

### PanCAM is Beta Cell Specific in the Pancreas

To further characterize the PanCAM protein, a soluble form of the extracellular domain was used in a screen for phage antibodies. Four human Fab fragments against extracellular portion of this protein were screened out from a phage antibody library (Knappik et al., J. Mol. Biol. 296(1):57-86 (2000). All of these Fabs recognized the recombinant protein on Western blot. Three of them recognized PanCAM on paraffin-fixed tissue slides.

Immunohistochemical (IHC) staining was performed on slides containing human heart, brain, kidney, liver, lung, pancreas, spleen and muscle tissues, and on pancreas tissue from monkey (Cynomolqous) and mouse. Among the human tissues tested, only the pancreas showed staining. The results clearly showed that the gene was expressed specifically in pancreatic islets of human, monkey and mouse.

In addition to the pancreas staining, the mouse anterior pituitary was also positive for IHC, with somewhat weaker and scattered staining. The pituitary staining explained the appearance of EST clones in brain libraries, as well as our ability to clone the mouse ortholog from brain cDNA.

To identify expression patterns of PanCAM at the cellular level, we performed RT-PCR on RNAs from multiple cell lines, including 293E, Cos7, mouse pancreatic endothelial cell (CRL 2279) (Arbiser et al., Proc. Natl. Acad. Sci. USA 94(3):861-6 (1997)), alpha cell (CRL 2350) (Powers et al., Diabetes 39(4):406-14 (1990)), beta cell from fat/fat mice (CRL2364)(derived from a mouse strain with a carboxypeptidase E mutation and defective in proinsulin processing and insulin secretion; Naggert et al., Nat Genet 10(2):135-42 (1995); Varlamov et al., Endocrinology 138(11):4883-92 (1997)), and beta cells from wild type mice (CRL2055) (Hamaguchi et al., Diabetes 40(7):842-9 (1991)). The results confirmed that PanCAM expression was beta cell specific.

Notably, beta cells from fat/fat mice yielded two RT-PCR products. Each of the RT-PCR bands was much fainter than the single band amplified from wild type beta cells. Sequence of the two RT-PCR products from the mutant cell line confirmed that the larger molecular weight band was same as that of the wildtype control, whereas the lower molecular weight band was an alternative splicing product. A cryptic splicing site was used to generate the shorter transcript, causing deletion of 3′ half of this exon and 4 downstream exons. Consequently, Ig domains 2 and 3 are missing in this open reading frame, while the downstream reading frame is maintained.

### Example 18

### PanCAM-Positive Cells Diminish During Development of Diabetes in NOD Mice

As a first step to test whether PanCAM plays a role in the development of type I diabetes, pancreata from NOD mice at ages of 4, 8, and 12 weeks, as well as retired breeders (animals with fully developed diabetes) were examined by IHC. At 4 weeks of age, the staining pattern was the same as in wild type mice, with the majority of cells in pancreatic islets being positive for PanCAM. At 8 weeks, when the mice started to develop pancreatitis, T cell invasion of islets was seen, and the number of PanCAM-positive cells started to diminish. At 12 weeks, there were more T cells and fewer PanCAM-positive cells in pancreas islets. In retired breeders, most of PanCAM-positive cells were lost, and T cells were observed where the islets used to be. The results demonstrated that overall expression of PanCAM was correlated with the development of diabetes in the NOD mice.

### Example 19

### Isolation of Beta Cells Using Anti-PanCAM Antibodies

Monoclonal or polyclonal antibodies specific for an extracellular portion of PanCAM (e.g., human PanCAM) are immobilized on a solid or semi-solid substrate and then contacted with dispersed pancreatic cells. Non-beta cells are removed by washing the substrate with an appropriate buffer. The bound beta cells are then recovered. Alternatively, the beta cells can be isolated by flow cytometry using anti-PanCAM antibodies. The purified beta cells are then entrapped into macroporous gelatin microcarriers (e.g., CulthiSpher-S) and transplanted to a patient having or at risk of having diabetes (see, e.g., Guerra et al., Biotechnol. Bioeng. 75(6):741-4 (2001).

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention. All publications and other references mentioned herein are incorporated by reference in their entirety. In case of conflict, the present specification, including definitions, will control. The materials, methods, and examples are illustrative only and not intended to be limiting. Throughout this specification and claims, the word “comprise,” or variations such as “comprises” or “comprising” will be understood to imply the inclusion of a stated integer or group of integers but not the exclusion of any other integer or group of integers. All ATCC deposits mentioned herein were made according to the provisions of the Budapest Treaty. All restrictions on the availability to the public of the above ATCC deposits will be irrevocably removed upon the granting of a patent on this application.

What is claimed is:

1. An isolated polypeptide comprising amino acid residues 68-594 of SEQ ID NO:12.

2. The polypeptide of claim 1, comprising SEQ ID NO:12.

3. The polypeptide of claim 1, comprising SEQ ID NO:26.

4. The polypeptide of claim 1, comprising SEQ ID NO:28.

5. The polypeptide of claim 1, comprising SEQ ID NO:8.

6. The polypeptide of claim 1, further comprising a heterologous sequence.

7. An isolated polypeptide comprising amino acid residues 4-513 of SEQ ID NO:8.

8. An isolated polypeptide comprising amino acid residues 535-708 of SEQ ID NO:8.

9. An isolated polypeptide comprising amino acid residues 535-633 of SEQ ID NO:26.

10. An isolated polynucleotide comprising a nucleotide sequence encoding the isolated polypeptide of claim 1.

11. The polynucleotide of claim 10, comprising nucleotides 202-1782 of SEQ ID NO:11.

12. The polynucleotide of claim 10, comprising SEQ ID NO:11.

13. The polynucleotide of claim 10, comprising SEQ ID NO:25.

14. The polynucleotide of claim 10, comprising SEQ ID NO:27.

15. The polynucleotide of claim 10, comprising nucleotides 196-2319 of SEQ ID NO:7.

16. The polynucleotide of claim 10, comprising SEQ ID NO:7.

17. The polynucleotide of claim 10, further comprising a transcription regulatory sequence operatively linked to said nucleotide sequence.

18. A vector comprising the polynucleotide of claim 10.

19. The vector of claim 18, wherein the vector is a plasmid.

20. The vector of claim 18, wherein the vector is a viral vector.

21. The vector of claim 18, selected from the group consisting of baculoviruses, adenoviruses, parvoviruses, herpesviruses, poxviruses, adeno-associated viruses, Semliki Forest viruses, vaccinia viruses, lentiviruses and retroviruses.

22. A host cell comprising the polynucleotide of claim 10.

23. The host cell of claim 22, wherein the host cell is selected from the group consisting of a bacterial cell, insect cell, yeast cell, plant cell and mammalian cell.

24. The host cell of claim 22, wherein the host cell is a human cell.

25. A method of producing a PanCAM polypeptide, comprising:
culturing the host cell of claim 22 in a medium under conditions in which said nucleotide sequence is expressed, and
recovering the polypeptide from the cell or from the culture medium.

26. An antibody that binds to the polypeptide of claim 1 or 7.

27. The antibody of claim 26, wherein the antibody is a monoclonal antibody.

28. The antibody of claim 26, wherein the antibody is a humanized or a fully human antibody.

29. A method of determining the presence of a PanCAM-encoding sequence in a sample, comprising:
contacting the sample with a nucleic acid probe comprising SEQ ID NO:3, 5, 6, 7, 9, 11, 25, or 27, or a portion thereof having at least 17 nucleotides, under high stringency hybridization conditions, and
detecting hybridization of said polynucleotide to a nucleic acid in the sample, wherein the occurrence of said hybridization indicates the presence of a PanCAM-encoding sequence in the sample.

30. A method of determining the presence of a PanCAM protein in a sample, comprising:
contacting the sample with the antibody of claim 26; and
detecting specific binding of said antibody to an antigen, wherein the occurrence of said specific binding indicates the presence of a PanCAM protein in the sample.

31. A method of identifying a compound that binds a PanCAM protein, comprising:
contacting the PanCAM protein with a test compound; and
detecting a complex formed by the PanCAM protein and the test compound, wherein the presence of the complex indicates that the test compound binds to the PanCAM protein.

32. A method of identifying a compound that modulates the activity of a PanCAM protein, comprising:
contacting the PanCAM protein with a test compound; and
determining the effect of the test compound on the activity of the PanCAM protein, whereas a change of the activity after the contacting step indicates that the test compound modulates the activity of the PanCAM protein.

33. A method of identifying a homolog of a human PanCAM gene, comprising:
hybridizing a nucleic acid library with a nucleic acid probe comprising SEQ ID NO:3, 5, 6, 7, 9, 11, 25, or 27, or a portion thereof consisting at least 17 nucleotides, under medium or high stringency hybridization conditions; and
determining whether the nucleic acid probe hybridizes to a nucleic acid sequence in the library,
wherein the nucleic acid sequence so hybridized is a homolog of a human PanCAM gene.

34. A method of diagnosing a disease condition in a subject, comprising:
comparing the amount or activity of a PanCAM protein in a tissue sample from the subject to the amount or activity of the PanCAM protein in a control sample,
wherein a significant difference in the PanCAM amount or activity of the tissue sample relative to the control sample indicates that the subject has a disease condition.

35. A method of diagnosing a disease condition in a subject, comprising:
comparing the amount of a PanCAM mRNA in a tissue sample from the subject to the amount of the PanCAM mRNA in a control sample,
wherein a significant difference in the PanCAM mRNA amount of the tissue sample relative to the control sample indicates that the subject has a disease condition.

36. The method of claim 34 or 35, wherein the tissue sample is a pancreatic tissue sample.

37. The method of claim 36, wherein the significance difference is a significant decrease, and the disease condition relates to diabetes.

38. The method of claim 34 or 35, wherein the disease condition relates to the central nervous system.

39. The method of claim 34 or 35, wherein the disease condition relates to the pituitary gland.

40. A method of identifying in a test cell the presence or absence of a genetic lesion or mutation characterized by at least one of: (i) aberrant modification or mutation of a gene encoding a PanCAM protein, (ii) mis-regulation of a gene encoding a PanCAM protein, and (iii) aberrant post-translational modification of a PanCAM protein, comprising:
separately hybridizing nucleic acids from the test cell and from a reference cell that lacks the genetic lesion or mutation with a nucleic acid probe comprising SEQ ID NO:3, 5, 6, 7, 9, 11, 25, or 27, or a portion thereof having at least 17 nucleotides, under high stringency hybridization conditions;
separately washing the nucleic acid hybrids under high stringency wash conditions to allow dissociation of the hybrids; and
determining whether the nucleic acid probe dissociates more readily from the nucleic acids of the test cell compared to the nucleic acids of the reference cell.

41. A method of treating a disease condition in a subject, comprising administering to the subject a composition that comprises the polynucleotide of claim 10, or the antibody of claim 26.

42. The method of claim 41, wherein the disease condition relates to the pancreas.

43. The method of claim 42, wherein the disease condition relates to the pancreatic beta cells.

44. The method of claim 43, wherein the disease condition relates to diabetes.

45. The method of claim 41, wherein the disease condition is selected from the group consisting of acute or chronic pancreatitis, pancreatic inflammation, pancreatic necrosis, exocrine insufficiency, pancreatic endocrine and hormonal imbalance, pancreatic tumors and associated cancers, and an auto-immune disorder which affects the pancreas.

46. The method of claim 41, wherein the disease condition relates to the central nervous system.

47. The method of claim 46, wherein the condition is selected from the group consisting of Alzheimer's disease, Parkinson's disease, senile dementia, migraine, epilepsy, neuritis, neurasthenia, neuropathy, neural degeneration and neural tumors.

48. The method of claim 41, wherein the disease condition is selected from the group consisting of pituitary hormone deficiency, growth hormone deficiency, and pituitary tumors.

49. A method of isolating beta cells from a dispersed pancreatic cell population, comprising:
providing an antibody specific for a PanCAM protein,
contacting the dispersed pancreatic cell population with the antibody under conditions that allow beta cells in the population to bind to the antibody, and
separating cells unbound by the antibody from beta cells bound to the antibody, thereby isolating beta cells.

50. The method of claim 49, wherein the dispersed pancreatic cell population is a bovine or swine pancreatic cell population.

51. A method of treating a human displaying a sign or symptom of diabetes or a pre-diabetic condition, comprising:
providing beta cells isolated by the method of claim 49, and
introducing the beta cells into the human.

52. The method of claim 51, wherein the beta cells are introduced subcutaneously in the human.

53. The method of claim 51, wherein the beta cells are encapsulated in a biological acceptable carrier.

54. An isolated polypeptide comprising SEQ ID NO:31.

55. An isolated polynucleotide comprising a nucleotide sequence encoding SEQ ID NO:31.

56. The polynucleotide of claim 55, comprising SEQ ID NO:32.
